(19) United States
(12) Patent Application Publication
(10) Pub. No.: US 2025/0041344 A1
(43) Pub. Date: Feb. 6, 2025

(54) GENE EDITING METHODS FOR MODULATING EXPRESSION OF ID-3, AN INHIBITOR OF DNA-BINDING TRANSCRIPTION FACTORS, THEREBY AFFECTING T-CELL FUNCTION

(71) Applicant: Hackensack Meridian Health, Inc., Edison, NJ (US)

(72) Inventors: Yi Zhang, Wallingford, PA (US); Wenbin MO, Nutley, NJ (US); Haihui XUE, Nutley, NJ (US)

(73) Assignee: Hackensack Meridian Health, Inc., Edison, NJ (US)

(21) Appl. No.: 18/793,364

(22) Filed: Aug. 2, 2024

Related U.S. Application Data
(60) Provisional application No. 63/530,414, filed on Aug. 2, 2023.

(51) Int. Cl. A61K 35/17; A61K 39/00; A61K 45/06; A61P 35/02; C07K 16/28; C12N 5/00; C12N 5/0783; C12N 9/22; C12N 15/11; C12N 15/86

(52) U.S. Cl. CPC A61K 35/17; A61K 39/4611; A61K 39/4631; A61K 39/464412; A61K 39/464413; A61K 39/464429; A61K 45/06; A61P 35/02; C07K 16/2818; C07K 16/2827; C07K 16/2878; C07K 16/2887; C12N 5/0018; C12N 5/0636; C12N 9/22; C12N 15/11; C12N 15/86; A61K 2239/13; A61K 2239/17; A61K 2239/21; A61K 2239/22; A61K 2239/38; C12N 2310/20; C12N 2501/2315; C12N 2501/2318; C12N 2501/2321; C12N 2740/10043

(57) ABSTRACT

The present disclosure provides gene editing methods for modulating the expression of an inhibitor of DNA-binding E-protein transcription factors, namely Id3, and thereby affecting T cell function. First, it provides experimental evidence that Id3 is critical to the persistence and function of tissue-infiltrating GVHD T cells in a mouse model. Id3 reduces chromatin accessibility (ChrAcc) of transcription factors (TFs) that drive T cell PD-1 transcription, differentiation and dysfunction. Id3 loss increases PD-1 expression and impairs tissue-infiltrating Th1 cells. Second, it provides proof-of-concept that targeting ID3 in human T cells using a CRIPSR/Cas9 knockout (KO) prevents xeno-GVHD but preserves the anti-leukemic activity of chimeric antigen receptor (CAR)-T cells. Third, it provides experimental evidence that ectopic expression of Id3 in engineered human CAR-T cells enhances the ability of these cells to eliminate tumors.

### CROSS-REFERENCE TO RELATED APPLICATIONS

This application claims the benefit of priority to U.S. provisional application 63/530,414 (filed Aug. 2, 2023) entitled “Gene-editing of human T cell ID3 and Allogeneic CAR-T Cell Therapy”, the contents of which are incorporated herein by reference in their entirety.

### SEQUENCE LISTING

The application contains a Sequence Listing which has been submitted electronically in .XML format and is hereby incorporated by reference in its entirety. Said .XML copy, created on Jul. 31, 2024, is named “129642-01202.xml” and is 9,875 bytes in size. The sequence listing contained in this .XML file is part of the specification and is hereby incorporated by reference herein in its entirety.

### FIELD OF THE INVENTION

The present invention relates generally to T cell biology. More specifically it relates to methods for modulating the expression of an inhibitor of DNA-binding E-protein transcription factors, namely Id3, and thereby affecting T cell function.

### BACKGROUND OF THE INVENTION

### T Cell Development

Lymphopoiesis takes place in specialized lymphoid tissues known as the central or primary lymphoid tissues, which are the bone marrow for most B cells and the thymus for most T cells. Precursors for both populations originate in the bone marrow; B cells complete most of their development there, while the precursors of most T cells migrate to the thymus where they develop into mature T cells. In the fetus and the juvenile, the central lymphoid tissues are the sources of large numbers of new lymphocytes, which migrate to populate the peripheral lymphoid tissues (also called secondary lymphoid tissues), such as lymph node, spleen and mucosal lymphoid tissue. In mature individuals, the development of new T cells in the thymus slows down, and peripheral T cell numbers are maintained by the division of mature T cells outside the central lymphoid organs. New B cells are continually produced from the bone marrow, even in adults. [Janeway's Immunobiology, 9^{th} Ed., Murphy K. and Weaver, C. Eds. Garland Science (2017) at p. 295].

The cells of the lymphoid lineage (B cells, T cells, and innate lymphoid cells, including NK cells) are all derived from common lymphoid progenitor cells, which themselves derive from the multipotent hematopoietic stem cells (HSCs) that give rise to all blood cells. Development from the precursor stem cell into cells that are committed to becoming B cells or T cells follows the basic principles of cell differentiation, i.e., properties that are essential for the function of the mature cell are gradually acquired, and properties that are more characteristic of the immature cell are lost. In the case of lymphoid development, cells become committed first to the lymphoid lineage, as opposed to the myeloid, and then to either the B cell or the T cell lineage. Notch signaling in thymic progenitor cells is essential to initiate the T cell specific gene expression program and commitment to the T cell lineage. Together T cell factor 1 (TCF1) and GATA2 initiate expression of several T lineage specific genes. A third transcription factor, Bc111b, is required to induce T-lineage commitment by restricting progenitor cells from adopting alternative fates. [Janeway's Immunobiology, 9th Ed., Murphy K. and Weaver, C. Eds. Garland Science (2017) at p. 297-317].

T cell development parallels that of B cells in many ways, including the orderly and stepwise rearrangement of antigen-receptor genes, the sequential testing for successful gene rearrangement, and the eventual assembly of a heterodimeric antigen receptor. T cell development has some features not seen for B cells, such as the generation of two distinct lineages of T cells expressing antigen receptors encoded by distinct genes, the γ:δ lineage and the α:β lineage. Developing T cells also undergo rigorous selection that depends on interactions with thymic cells and that shapes the mature repertoire of T cells to ensure self MHC restriction as well as self-tolerance. [Janeway's Immunobiology, 9th Ed., Murphy K. and Weaver, C. Eds. Garland Science (2017) at p. 315].

Phenotypic and functional analysis of CD4+ and CD8+ T cells from blood, lymphoid and mucosal sites employed to study T cells isolated from human tissues obtained from organ donors to gain insights into how T cell subsets are distributed and functionally maintained in humans revealed that the organization, differentiation and maintenance of human T cells was found to be strikingly tissue intrinsic. [Sathaliyawalla, T. et al. Immunity (2013) 38: 187-97]. This multidimensional analysis revealed distinct compartmentalization of naïve, effector and memory CD4+ and CD8+ T cell subsets intrinsic to the tissue site that was remarkably consistent in diverse individuals. Memory CD4+ T cells represent the majority subset in mucosal tissues and accumulate in lymphoid tissue throughout life. CD8+ T cell subsets, by contrast, are maintained as naïve cells in lymphoid compartments over decades, with memory CD8+ T cells mainly in mucosal sites and terminal effector cells confined to circulation. Memory T cells in all tissues specifically upregulate CD69 expression, a marker of T cell receptor (TCR)-mediated signaling, which distinguishes tissue-resident from circulating populations. Functionally, the majority of tissue-resident T cells were quiescent or IL-2-producing memory CD4+ T cells, followed by IFN-γ-producing memory CD8+ T cells, with IL-17 production confined to memory CD4+ T cells in mucosal compartments.

### T Cell Differentiation

T cells acquire their functional properties in two main phases. The first occurs in the thymus, as T cells transit through successive stages that install the gene expression programs that will run at steady state. The second phase of differentiation occurs in the periphery after exposure to signals that occur during an immune response. These signals activate accessible but latent sub-routines that are kept in check prior to the initiation of the immune response. Both processes depend on the activity of E protein transcription factors and their antagonists, the Id factors. In many different T cell lineages, E proteins have a context-dependent use; T cell receptor (TCR) signaling and Id3 activity, in collaboration with other extracellular signals, creates those contexts. While TCR signaling is required for peripheral CD4 T cell differentiation, the specific functional pathways accessed in the periphery are very sensitive to the cytokine milieu. In contrast, the progression of T cell precursors into different pathways in the thymus appears to be driven more by TCR signal strength. In both cases, TCR-dependent upregulation of Id3 is important for allowing changes in chromatin remodeling and gene expression that are needed to restrict E protein activity to the appropriate targets. [Anderson, MK. Front. Immunol. (2022) 13: doi.org/10.3389/fimmu.2022.956156].

Conventional CD4 T cells emerge from the thymus as “naïve” cells ready for activation. The functional T helper cell differentiation pathways they take upon antigen encounter depends on the types of inflammatory molecules produced during the innate immune response [Id., citing Martinez-Sosa, P. and Mendoza, L. Biosystems (2013) 113 (2): 96-103]. Each T helper cell subset is dependent on a specific “master regulator” transcription factor that directly induces the effector genes of each program [Id., citing Hirahara, K. et al. J. Allergy Clin. Immunol. (2013) 131 (5): 1276-87]. The Th17 lineage, characterized by secretion of IL-17A, IL-17F, and IL-22, is triggered by the innate response to bacteria and fungi. RORγt (Rorc) is the Th17 master regulator. Viruses and other intracellular pathogens induce differentiation into the T-bet (Tbx21) dependent Th1 pathway, leading to IL-2, TFNα, and IFNγ production. Helminth infection induces the Th2 fate, leading to secretion IL-4, IL-5, and IL-13, under the control of GATA3 [Id., citing Butcher, M J and Zhu, J. Fac. Rev. (2021) 10: 30].

Other Th subsets generated in the periphery include Bcl6-driven T-follicular helper cells (Tfh) [Id., citing Hatzi, K., et al. J. Exp. Med. (2015) 212 (4): 539-53], specialized for B cell help in the germinal center, and induced T-reg cells, which, like thymic-derived T-regs, depend on FoxP3 [Id., citing Georgiev, P. et al. J. Clin. Immunol. (2019) 39 (7): 623-40]. In addition to playing unique roles in immunity, Th subsets also have pathogenic impacts when dysregulated [Id., citing Nakayama, T. et al. Annu. Rev. Immunol. (2017) 35: 53-84]. In general, Th1 and Th17 cells contribute to autoimmune pathology, Th2 cells are largely responsible for allergic reactions, and T-regs inhibit anti-cancer immunity [Id., citing Dobrzanski, MJ. Front. Oncol. (2013) 3: 63; Sarkar, T. et al. Curr. Res. Immunol. (2021) 2: 132-41]. Most Th subsets retain plasticity after activation, and some can transdifferentiate from one type to another [Id., citing Hirahara, K. et al. J. Allergy Clin. Immunol. (2013) 131 (5): 1276-87]. Additional Th subsets continue to be identified, including Th22, Th9, Tfh13, and Tr1 cells, suggesting that the networks controlling these effector functions are dynamic, and represent more of a physiological state than a committed fate [Id., citing Plank, M W, et al. J. Immunol. (2017) 198 (5): 2182-90; Schmitt, E. et al. Trends Immunol. (2014) 35 (2): 61-6; Gowthaman, U. et al. Science (2019) 365 (6456): 1-22].

### Transcriptional Control of Th Differentiation

Differentiation of naïve CD4 T cells into the Th subsets is coordinated by several sets of signal-dependent transcription factors [Id., citing Rogers, D. et al. Cell Rep. (2021) 37 (9): 110064]. Triggering of the αβ TCR and co-stimulatory receptors leads to activation of NFkB, NFAT, and IRF transcription family members, as well as upregulation of APi transcription factor family members such as BATF and Jun [Id., citing Iwata, A. et al. Nat. Immunol. (2017) 118 (5): 563-72; Carr, T M et al. Nat. Commun. (2017) 8 (1): 301]. Cytokine receptor signaling leads to the activation of different sets of transcription factors, most notably members of the STAT and SMAD families [Id., citing Yoo, S A et al. Nat. Immunol. (2019) 20: 1348-59; Malhotra, N. et al. J. Biol. Chem. (2010) 285 (38): 29044-8]. BATF, IRF4, and the cytokine-responsive factors recruit chromatin remodeling enzymes that provide access to genes of specific Th subsets, while restricting access to genes of alternative Th subsets [Id., citing Iwata, A. et al. Nat. Immunol. (2017) 18 (5): 563-72; Wei, G. et al. Immunity (2009) 30 (1): 155-67]. After chromatin remodeling, the master regulators are induced, providing the final key needed for functional activation during the immune response.

The basic Helix-Loop-Helix (bHLH) proteins represent a well-known class of transcriptional regulators. Many bHLH proteins act as heterodimers with members of a class of ubiquitous partners, the E-proteins. A widely-expressed class of inhibitory heterodimer partners—the Inhibitor of DNA-binding (ID) proteins—also exists. [Wang, L-H and Baker, NE. Dev. Cell (2015) 35 (30: 269-80)

The E-proteins and Id proteins are involved in regulation of the naïve CD4 T cell state, and in the differentiation of Th2, Th17, and T-reg cells (Anderson, MK. Front. Immunol. (2022) 13: 956156, citing Zook, E C et al. Sci. Immunol. (2018) 3 (22): 1-17; Miyazaki, M. et al. Nat. Immunol. (2011) 12 (10): 992-1001; Zhou, H. et al. Mol. Med. Rep. (2017) 16 (6): 9086-94). In general, E protein activity is regulated post-translationally by Id proteins, which sequester them in inactive dimers. The requirement for E proteins for Th17 differentiation has been especially well studied. A comprehensive study showed that mice carrying a conditional double HEB/E2A deletion on a CD4-Cre background had a profound defect in Th17 development in vitro, and compromised immune function in vivo, using both autoimmunity and infection models [Id., citing Zhang, J. et al. Science (2004) 305 (5688): 1286-9]. This study also showed that HEB and E2A can directly bind and activate the Rorc locus, but only in the context of Th17 cells, not in naïve CD4 T cells. Studies of Id3-deficient mice suggest that E proteins restrain the Th2 and Tfh lineages and promote the Th 9 lineage, whereas Th1 cells appear to require Id proteins and to be E protein independent [Id., citing Zhang, J. et al. Science (2004) 305 (5688): 1286-9; Nakatsukasa, J. et al. Nat. Immunol. (2015) 16 (10): 1077-84; Mityazaki, M. et al. Genes Dev. (2015) 29 (4): 409-25; Han, X. et al. Eur. J. Immunol. (2019) 49 (3): 476-89]. T-regs require both Id3 and E2A in a sequential manner. TGFβ induces transient expression of Id3, which is needed to prevent repression of the FoxP3 promoter [Id., citing Maruyama, T. et al. Nat. Immunol. (2011) 12 (1): 86-95]. This repression is not mediated directly by E proteins, but rather results from E protein-mediated upregulation of GATA3. Subsequently, E2A activity is required to directly activate the FoxP3 promoter. However, if E2A levels are too high, FoxP3 expression becomes unstable in T-regs, emphasizing the importance of transcription factors levels in maintaining stable outcomes [Id., citing Rauch, K S et al. Cell Rep. (2016) 17 (110: 2827-36).

### Id2 Regulates the Innate/Adaptive Fate Choice in Early T Cell Precursors

The earliest T cell progenitors (ETPs) to enter the thymus are not yet committed to the T-cell lineage and have alternative fates available to them depending on their access to microenvironmental signals. One of the key molecular switches that must be flipped to gain access to the T cell pathway is to increase E protein activity. This occurs in at least two different ways. The first is upregulation of E proteins at the mRNA level, and the second is the downregulation of Id2 [Id., citing Rothenberg, EV. Et al. Nat. Rev. Immunol. (2008) 8 (1): 9-21]. Id2 is a critical mediator of the innate/adaptive lineage split [Id., citing Zook, E C et al. Sci. Immunol. (2018) 3 (220): 1-17; Muyazaki, M. et al. Immunity (2017) 46(5): 818-34]. ETPs express “legacy genes”, thus termed because they are expressed in hematopoietic stem cells [Id., citing David-Fung, E S, et al. Dev. Biol. (2009) 325 (2): 444-67]. ETP legacy genes include Id2, the Ets protein PU.1, and the Class II bHLH factor SCL. All three of these factors can act in opposition to T-lineage commitment: PU.1 drives expression of myeloid and B cell genes [Id., citing Anderson, M K et al. Development (1999) 126 (14): 3131-48], SCL can re-direct E proteins to stem cell gene loci and away from T cell gene loci [Id., citing Gerby, B. et al. PLoS Genet. (2014) 10 (12): e1004768], and Id2 interferes with E protein activity. E protein activity is essential for the expression of Rag recombinase genes, which are necessary for the generation of TCRs and thus T cells [Id., citing Miyazaki, K. and Miyazaki, M. Front. Immunol. (2021) 12: 659761]. Unlike Id3, Id2 is subject to degradation in a cell cycle-dependent manner [Id., citing Lasorella, A. et al. Nature (2006) 442 (7101): 471-4; Sullivan, J M et al. Stem Cells (2016) 34 (5): 1321-31]. Downregulation of PU.1 and upregulation of Bclllb in early T cell development results in the cessation of Id2 mRNA expression, which allows upregulation of T-lineage E protein target genes [Id., citing Anderson, M K et al. Immunity (2002) 16 (20: 285-96; Hosokawa, H. et al. Nat. Immunol. (2018) 19 (120: 1427-40). Conversely, Id2 expression is maintained in mature innate cells including ILCs, NK cells, and myeloid cells, and appears to support the maintenance of lineage fidelity.

### Notch Signaling Shifts the E Protein-Id2 Balance to Allow T Cell Development

As ETPs enter the thymus, they are exposed to Delta-like (Dll) ligands of Notch receptors, resulting in strong Notch signaling. Notch signaling is indispensable for T cell specification and lineage commitment, acting upstream of an elegant cascade of transcription factors that inhibits alternative fates and induces T-cell genes [Id., citing Rothenberg, EV. Curr. Opin. Syst. Biol. (2019) 18: 62-76]. While Notch regulates a wide swath of important target genes, it plays an important role in T-lineage commitment by shifting the balance between Id and E protein activity in ETPs, in three complimentary ways. First, Notch redirects PU.1 away from Id2 and towards more T-lineage friendly genes [Id., citing Del Real, M M and Rothenberg, EV. Development (2013) 140 (6): 1207-19]. Secondly, Notch upregulates the E protein HEBAlt, increasing overall E protein availability [Id., citing Wang, D. et al. J. Immunol. (2006) 177 (1): 109-19]. Thirdly, Notch directly upregulates Bcl11b, which downregulates Id2 at the transcriptional level [Id., citing Hosokawa, H. et al. Nat. Immunol. (2018) 19 (120: 1427-40). These direct cells permanently away from Id2-dependent ILCs and into the T-cell lineage.

### TCR Signal Strength Determines Lineage Outcomes During the Intrathymic T Cell Lineage Choices

Once cells have been switched onto the T-lineage track, they progress towards the first “checkpoint” of T cell development. There are two main checkpoints that occur during T cell development, so called because they serve as testing of the cells for functional TCR rearrangement and function. During the first checkpoint, the TCRβ chain pairs with the pre-Ta chain to form a pre-TCR. The only requirement for the pre-TCR to allow “passage” through the checkpoint is for it to complex with CD3 chains and translocate to the cell membrane long enough to invoke a weak set of signaling cascades [Id., citing Dutta, A. et al. Trends Immunol. (2021) 42 (80: 735-50). Alternatively, the cell can rearrange and express a TCR composed of TCRγ and TCRδ chains. In this situation, the γδ TCR/CD3 complex is stably expressed on the surface, transmitting a stronger signal than that transduced by the pre-TCR, which directs cells away from the αβ T cell fate and into the γδ T cell fate [Id., citing Haks, M C et al. Immunity (2005) 22 (5): 595-606; Hayes, S M et al. Immunity (2005) 22(5): 583-93]. After commitment to the αβ T cell lineage, cells expressing αβ TCRs are subjected to a second “checkpoint” which vets these TCRs for their ability to bind to MHC/peptide and assesses the affinity of the interaction. As with the first checkpoint, this signal also serves as a lineage branch point, with cells experiencing lower and briefer TCR signaling adopting the CD8 fate, and cells experiencing longer and stronger TCR signaling progressing into the CD4 T cell lineage [Id., citing Karmimi, M M et al. Nat. Commun. (2021) 12 (1): 99]. This paradigm also applies to committed γδ T cells that progress along the IFNγ-producing γδT1 fate or the γδT17 fate [Id., citing Zarin, P. et al. Immunol. Cell Biol. (2018) 96 (9): 994-1007]. Engagement of strong γδ TCR ligands in conjunction with co-stimulatory molecules results in strong TCR signaling and the γδT1 developmental outcome, whereas a less strong TCR signal leads to the γδT17 fate [Id., citing Sumaria, N. et al. Cell Rep. (2017) 19 (12): 2469-76; Ribot, J C et al. Nat. Immunol. (2009) 10 (4): 427-36; Chen, E L Y et al. Cell Rep. (2021) 35 (10: 109227). All these lineage choices are intimately associated with the balance between Id3 and E proteins [Id., citing Jones, ME and Zhuang, Y. Immunol. Res. (2011) 49 (1-3): 202-15; Anderson, M K and Selvaratnam, J S. Immunol. Rev. (2020) 298 (1): 181-97].

Translation of TCR Signal Strength into Id3 Activity Modulates E Protein Target Gene Accessibility

As in peripheral CD4 T cells, TCR signaling in early precursors leads to upregulation of Id3, and a pause in E protein activity allows chromatin remodeling and shifting of E protein target availability. There may also be a role for TCR signal strength during T helper cell differentiation, particularly in combination with cytokine signaling [Id., citing Bhattacharyya, ND and Feng, CG. Front. Immunol. (2020) 11: 624]. However, there is a clear hierarchy of TCR signal strength that is induced at each checkpoint in thymic T cell development [Id., citing Zarin, P. et al. Cell Immunol. (2015) 296 (1): 70-5]. During T cell development, TCR signaling may shift the balance between Id3 and E proteins to different degrees, allowing retention of E protein occupancy on some sites but not others. E2A and HEB are direct regulators of most of the genes needed for assembly of the TCR genes and formation of the pre-TCR [Id., citing Miyazaki, K. et al. Sci. Immunol. (2020) 5 (51): 1-15; Schwartz, R. et al. Proc. Natl Acad. Sci. USA (2006) 103 (26): 9976-81]. Id3 is also induced in response to aPTCR signaling at the double positive (DP, CD4+CD8+) stage, and is necessary to overcome the gatekeeper function of E proteins at the DP to single positive (SP) transition [Id., citing Bain, G. et al. J. Exp. Med. (1999) 190 (11): 1605-16; Jones, ME and Zhuang, Y. Immunity (2007) 27 (6): 860-70]. However, past this checkpoint, E proteins are required for the generation of CD4 SP cells [Id., citing Jones-Mason, M E et al. Immunity (2012) 36 (3): 348-61]. E proteins also regulate genes in γδ-T committed cells that dictate functional programming, including Tcf7 [Id., citing Fabi, S P et al. Cell Rep. (2021) 34 (3): 108716]. Using ATAC-seq, which detects open chromatin and predicts the presence of transcriptional complexes [Id., citing Hosoya, T. et al. Sci. Rep. (2018) 8 (1): 5605], Hosoya et al. showed that the loci for both γδ-lineage and αβ-lineage genes were accessible in DN thymocytes. However, as cells transitioned from the DN to the DP stage and then to the CD4 and CD8 stages, cis-regulatory elements with predicted binding by the key γδ-lineage factor Sox13 showed a dramatic loss of accessibility. Likewise, predicted HEB sites shifted in accessibility according to the stage of αβ T cell development, consistent with Id3-facilitated chromatin remodeling at these transitions.

### Id3-Facilitated Shifts in E Protein Targets

Like E proteins, Id3 is used widely in different contexts outside of T cell development [Id., citing Hai, Y. et al. Discovery Med. (2015) 19 (105): 311-25; Micheli, L. et al. J. Cell Physiol. (2015)230 (12): 2881-90]. Release of E proteins from specific sites likely depends on both the Id3/E protein ratio and the availability of E protein binding partners. For instance, the downregulation of Notch1 in response to pre-TCR signaling would be predicted to increase the disengagement of E proteins from sites that require both Notch factors and E proteins, but not from other sites that maintain the core T-lineage program. E proteins themselves are important mediators of chromatin remodeling, interacting directly with both positive and negative regulators of chromatin configuration such as p300, CHD4, LSD1, and PRC2 [Id., citing Hyndman, B D et al. Biochim. Biophys Acta (2012) 1819 (5): 375-81; Williams, C J et al. Immunity (2004) 20(6): 719-33; Teachenor, R. et al. Mol. Cell Biol. (2012) 32 (9): 1671-82; Yoon, S J et al. Nat. Commun. (2015) 6: 6546; Yi, S. et al. Development (2020) 147 (23): 1-14]. In this context, chromatin remodeling does not indicate simply a shift between “open” and “closed” configurations, but also includes the transition from “poised” to “active” states [Id., citing Dutta, A. et al. Int. Rev. Immunol. (2021) 1-9], which may be mediated in part by fresh access to new binding partners that become available after the transition. Furthermore, the plasticity of CD4 T cell subsets suggests that lineage-specifying E protein sites remain accessible during and after CD4 T cell differentiation [Id., citing Lu, K T et al. Immunity (2011) 35 (40: 622-32). A comprehensive understanding of global E protein occupancy changes that occur during these processes awaits further studies.

### GVHD in Adoptive Cell Therapy

Allogeneic hematopoietic stem cell transplantation (alloSCT), a robust form of adoptive cell therapy (ACT) that has been tremendously effective in the treatment of leukemia, is a potentially curative therapy for malignant and nonmalignant disorders of hematologic cells. A3 T cells in the donor graft that recognize recipients as “non-self” (alloreactive) promote engraftment by attacking host hematopoietic and immune cells, and in the application of alloSCT for neoplastic disease, such T cells can kill malignant blood-lineage cells, mediating a graft-versus-leukemia (GVL) effect. However, alloreactive T cells also can attack normal host tissues, causing graft-versus-host disease (GVHD).²

Acute GVHD (aGVHD) results from an immune-mediated attack on recipient tissue by donor T cells contained in or developed from a graft that recognize and bind their cognate ligands on recipient APCs in combination with essential second signals on the APC surface. The resulting cytolytic T cell response is executed via perforin, granzymes and Fas ligand, with inflammatory cytokines augmenting this response. [Sonntag, K. et al. J. Autoimmunity 62 (2015): 55-66].

Graft content in T-cell subsets has been correlated to the risk of aGvHD, in particular naïve CD45RA+CD4+ T cells [Duti, S. et al. J. Immunology (2007) 179: 6547-54], CD4−iNKT cells [Chaidos, A. et al. Blood (2012) 119 (21): 5030-36; Coman, T. et al. Oncoimmunology (2018) 7 (1): e1470735; Rubio, M-T et al., Leukemia (2017) 31: 903-12, citing Chang, Y J, et al. J. Clin. Immunol. (2009) 29: 696-704], naïve and central memory CCR7+CD4+ T cells [Rubio, M-T et al., Leukemia (2017) 31: 903-12, citing Yakoub-Agha, I. et al. Leukemia (2006) 20: 1557-65], or CD 8 effector memory T cells (CD45RA-CD62L−) [Rubio, M-T et al., Leukemia (2017) 31: 903-12, citing Loschi, M. et al. Biol. Blood Marrow Transplant (2015) 21: 569-74].

The pathophysiology of chronic GVHD (cGVHD) is less well understood. It is typically an autoimmune-like syndrome developing gradually involving donor-derived T cells primed on APCs. It is thought to involve three main pathological mechanisms: autoantibody production, systemic fibrosis, and defects in thymic function. Autoimmune systemic sclerodermatous GVHD was reported in NSG mouse cohorts that were humanized using exclusively CD34+-selected, CD3+-depleted stem cell grafts (hhCD34+NSG). [Sonntag, K. et al. J. Autoimmunity 62 (2015): 55-66].

Graft-versus-host disease (GVHD) is caused by host-reactive donor T cells that infiltrate and damage peripheral tissues in recipients after allogeneic hematopoietic stem cell transplantation (allo-HSCT).¹⁻³ These organ-infiltrating donor T cells produce a plethora of inflammatory cytokines and cytotoxic molecules to mediate tissue injury.⁴⁻⁹ Tissue inflammation frequently emerges despite the concurrent use of immunosuppressive agents targeting systemic T cells; early treatment resistance is common.³ Graft versus Host Disease (GVHD) syndromes therefore pose a significant threat of morbidity, escalated and prolonged immunosuppressive therapy, organ dysfunction, impaired quality of life, and ultimately an increased risk for mortality.

Although the initiation of the alloreactive T cell response has been well studied, much less is known about how GVHD is established and maintained after early alloreactive T cell activation.²

T cell infiltration into secondary lymphoid organs and non-lymphoid tissues is central to T cell function and occurs during homeostatic tissue surveillance, as well as during T cell-driven immunopathology (including auto- and allo-immune disorders) and T cell-mediated anti-tumor immune attack. Organ infiltration by donor T cells is critical to the development of acute graft-versus-host disease (aGVHD) in recipients after allogeneic hematopoietic stem cell transplant (allo-HCT). During aGVHD, donor T cells first populate secondary lymphoid organs, where they undergo allo-antigen priming, and then home towards and infiltrate non-lymphoid GVHD-target organs. Upon infiltration, these cells induce the immunologic and clinical manifestations of aGVHD, including wide-spread organ damage.¹

Persisting alloreactive donor T cells in target tissues are a determinant of graft-versus-host disease (GVHD). While tissue-infiltrating donor T cells are initially activated in the secondary lymphoid organs, they are expanded and maintained locally within the resident tissues during the effector phase.^{1-3,10}

Once established, GVHD is largely maintained locally in tissues by a TCF-1+ progenitor-like T cell population.² These tissue-infiltrating donor T cells express a tissue-resident memory-like phenotype,¹ and may be locally maintained by TCF-1+ progenitor-like T cells.² The transcriptional regulators that control the persistence and function of tissue-infiltrating T cells are not understood.

### Chimeric Antigen Receptor (CAR)-T Cell Immunotherapy

An immunotherapy using autologous T cells endowed with chimeric antigen receptors (CARs) has emerged as a powerful approach for treating cancer. Cytotoxic T-cells are engineered to express a CAR targeted to a tumor specific antigen expressed on the surface of tumor cells. These engineered CAR T-cells are then cytotoxic to cells expressing the tumor specific antigen. Autologous CAR-T cell therapy does not carry a significant risk of graft-versus host disease because the cells are patient-derived, and theoretically, such cells would have undergone thymic selection, which is aimed at eliminating auto-reactive T-cell clones. [Sanber, K. et al. Br. J. Haematology (2021) 195: 660-68, citing Cheng, M. and Anderson, MS. Nat. Immunol. (2018) 19: 659-64] Nevertheless, anti-CAR immunity has been documented clinically [Wagner, DLl et al. Nat. Rev. Clin. Oncol. (2021) 18 (6): 373-93]. In some patients, genetically engineered autologous T cells might not be effective due to T-cell dysfunction, which is a hallmark of many cancers and is associated with multiple mechanisms of immunosuppression derived from the tumor microenvironment (TME). [Depil, S. et al. Nature Reviews Drug Discovery (2020) 19: 185-99]. The biological characteristics of autologous T cells also can be adversely affected by previous lines of treatment leading to product failure.

Allogeneic approaches theoretically potentially could overcome these limitations. However, the administered allogeneic T cells may cause life-threatening GVHD, and the allogeneic T cells may be rapidly eliminated by the host immune system, limiting their antitumor activity.

Adoptive cell therapy (ACT) represents a logical extension of alloHCT treatments for leukemia. While ACT currently involves the isolation and infusion of T lymphocytes, ACT using other effector cell types, including 7⁶ T cells, natural killer (NK) cells, NK T cells, and invariant NK T cells, is also an area of active investigation. [Garber, H R et al. Mol. Cell Ther. (2014) 2:25, citing Deniger, D C et al. Mol. Ther. (2013) 21: 638-47; Shimasaki, N. and Camana, D. Methods Mol. Biol. (2013) 969: 203-20; Sangiolo, D. et al. Expert Opin. Biol. Ther. (2009) 9: 831-40]. Generally, T cells must undergo ex vivo manipulation and expansion prior to administration, an extensive process where cells may spend a month or more in culture, to generate large numbers of T-cells that are specific for one or more tumor antigens from an infrequent and polyclonal population. Genetic modification of T-cells bypasses these lengthy enrichment protocols by direct transfer of either TCR α and β chains or a chimeric antigen receptor (CAR), thereby endowing lymphocytes with a secondary, engineered specificity.

### Role of ID3 in Alloreactive Cells

As explained above, Id3 is a helix-loop-helix (HLH) factor that controls T cell development and function through antagonizing the function of the E box binding family of transcription factors, which are master regulators of thymic development and antigen-driven T cell responses. Id3 promotes CD8+ T cell-, γδ T cell- and iNKT cell-development, but antagonizes CD4+ T cell fate determination.¹¹⁻¹⁴ Id3 also regulates antigen-specific CD8+T cell memory formation, memory recall response and effector survival,¹⁵⁻¹⁷ and is associated with CD4+ memory Th1 cells.¹⁸ Additionally, Id3 expression may identify precursor exhausted CD8+ T cells during chronic infections.¹⁹ However, the precise role of Id3 in alloreactive T cells, specifically tissue-infiltrating pathogenic T cells, has not been previously defined.

The present disclosure provides experimental evidence of transcriptional regulation of T cell function by Id3. First, the present disclosure provides experimental evidence that Id3 is critical to the persistence and function of tissue-infiltrating GVHD T cells in a mouse model. Id3 reduces chromatin accessibility (ChrAcc) of transcription factors (TFs) that drive T cell PD-1 transcription, differentiation and dysfunction. Id3 loss increases PD-1 expression and impairs tissue-infiltrating Th1 cells. Second, it provides proof-of-concept that targeting ID3 in human T cells using a CRIPSR/Cas9 knockout (KO) prevents xeno-GVHD but preserves the anti-leukemia activity of tumor-reactive T cells. Third, given the role of the PD-1 pathway in repressing tumor immunity, programming Id3 in tumor reactive T cells may enhance their therapeutic efficacy by rendering them resistant to PD-1 pathway-mediated suppression. Fourth, it provides experimental evidence that ectopic Id3 expression in engineered CAR-T cells enhances the ability of the CAR-T cells to reduce lymphoma and leukemia.

### SUMMARY OF THE INVENTION

According to one aspect, the present disclosure provides an immunotherapy method for treating a recipient subject with a hematologic cancer comprising administering to the recipient subject an activated and expanded purified population of genetically engineered CD3+ T cells derived from a healthy donor, wherein the genetic engineering of the donor CD3+ T cells in vitro reduces expression of Id3, an inhibitor of DNA-binding E-protein transcription factors, by at least 25%; and wherein the method poses a decreased risk of graft versus host reaction while preserving graft versus tumor immunity in the recipient subject.

According to some embodiments of the immunotherapy method, the administering is by infusion. According to some embodiments, the subject is a mammal; or the subject is a human.

According to some embodiments, the hematologic cancer is a leukemia, a myelodysplastic neoplasm, a myeloma, or a lymphoma.

According to some embodiments, the donor T cells are allogeneic to the recipient subject.

According to some embodiments, the CD3+ T cells are purified from mononuclear cells collected from umbilical cord blood or adult peripheral blood; and the CD3+ T cells comprise CD4+ T cells, CD8+ T cells or both; and the CD3+ T cells comprising the edited Id3 gene are expanded and activated in vitro in presence of a cytokine.

According to some embodiments, the cytokine is selected from the group consisting of IL-2, IL-7, IL-15, IL-18, IL-21, or a combination thereof.

According to some embodiments, the reducing of the expression of the Id3 gene of the CD3+ T cells is accomplished by CRISPR/Cas9.

According to some embodiments, the activated and expanded purified population of genetically engineered CD3+ T cells comprising CD4+ T cells, CD8+ T cells or both comprising the edited Id3 gene is characterized by an improved ability to secrete effector cytokines, an improved cytotoxicity, or both against tumor cells compared to a control population of mononuclear cells.

According to another aspect, the present disclosure provides an immunotherapy method for treating a recipient subject with a hematologic cancer comprising (a) genetically engineering a population of CD3+ T cells derived from a healthy donor: to express a chimeric antigen receptor (CAR) that specifically binds a tumor antigen; and to ectopically express Id3 (Id30E); (b) activating and expanding the purified population of genetically engineered CD3+CAR−, IdOE T-cells of (a); and (c) administering the activated and expanded purified population of genetically engineered population of CD3+CAR, IdOE T-cells of (b) wherein the cell population is characterized by: a lower frequency of terminally exhausted Tcells of phenotype PD-1+TIM3+; or a higher frequency of progenitor exhausted cells (TPEX) of phenotype PD1+TIM3−; or an enhanced persistence and enhanced ability to expand in vitro; or a higher frequency of cells of a central memory cell phenotype (CD62+CD45RA−) upon ex vivo culture in IL-2, IL-7 and IL-15; or an enhanced ability to produce IL-2 and to proliferate upon antigen challenge in ex vivo culture; or augmented memory protection against tumor challenge in a mouse leukemia model; or improved overall survival; or a combination thereof, compared to control CAR-T cells.

According to some embodiments, (a) the genetic engineering to express a CAR comprises transducing the CD3+ T cells with a retroviral vector comprising a nucleic acid encoding a synthetic CAR to stably express the CAR; and (b) the CAR comprises an extracellular antigen recognition domain, a spacer/hinge region and transmembrane domain, and an intracellular signal transduction domain; and (c) the therapeutic dose of the CAR-T cells is about 1×10E6 to 20×10E6 CAR-T cells/m2 body surface area.

According to some embodiments, the extracellular antigen recognition domain of the CAR comprising an scFv fragment derived from a monoclonal antibody binds specifically to CD19, CD20, CD22, CD33, or CD30; and the intracellular signal transduction domain of the CAR comprises a CD3ζ activation chain and one or more costimulatory molecules.

According to some embodiments, the costimulatory molecule comprises 4-1BB.

According to some embodiments the immunotherapy method further comprises administering a short course of chemotherapy to reduce the T cell population of the subject prior to the administering of the population of CAR-T ID3OE cells.

According to some embodiments, the human CAR-T cells engineered to ectopically express Id3 have an enhanced ability to eliminate tumors compared to a CAR-T cell control that does not ectopically express Id3.

According to some embodiments, the administering is by infusion.

According to some embodiments, the subject is a mammal; or the subject is a human.

According to some embodiments of the immunotherapy method, the hematologic cancer is a leukemia, a myelodysplastic neoplasm, a myeloma, or a lymphoma.

According to some embodiments of the immunotherapy method, the donor T cells are allogeneic to the recipient subject.

According to some embodiments of the immunotherapy method, a. the CD3+ T cells are purified from mononuclear cells collected from umbilical cord blood or adult peripheral blood; the CD3+ T cells comprise CD4+ T cells, CD8+ T cells or both and the CD3+ T cells are expanded and activated in vitro in presence of a cytokine selected from IL-2, IL-7, IL-15, IL-18, IL-21, or a combination thereof.

According to some embodiments, the immunotherapy method further comprises administering an additional agent. According to some embodiments, the additional agent comprises: an approved immune checkpoint inhibitor at a dose standard for the cancer indication, or rituxuximab (anti-CD20); or alemtuzumab (antiCD52); or epratuzumab (anti-CD-22); or a clinical grade alpha-1-antitrypsin.

According to some embodiments, the immune checkpoint inhibitor is an anti-PD-1 inhibitor; an anti-PD-L1 inhibitor atezolizumab, or an anti-CTLA-4 inhibitor.

According to some embodiments, the anti-PD-1 inhibitor is lambrolizumab/pembrolizumab or nivolumab. According to some embodiments, the anti-PDL-1 inhibitor is atezolizumab. the anti-PDL-1 inhibitor is atezolizumab. According to some embodiments, According to some embodiments, the anti-CTLA-4 inhibitor is ipilimumab.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 comprising FIG. 1A, FIG. 1B, FIG. 1C, FIG. 1D, FIG. 1E, FIG. 1F and FIG. 1G shows that Id3 maintains the GVHD-inducing capacity of alloreactive T cells. Balb/c mice were given total body irradiation (TBI, 4.5 Gy on day −1 and 4 Gy on day 0) followed by infusion of 5×10⁶ B6×B6/SJL F1 mouse (CD45.1+CD45.2+) T cell-depleted bone marrow (TCD-BM) alone or together with 5×10⁵ B6 (CD45.1-CD45.2+) naïve WT or Id3-cKO CD4+ T cells. FIG. 1A is a survival curve depicting survival of Balb/c recipients. FIG. 1B shows cutaneous GVHD in Balb/c mice receiving WT and Id3-cKO CD4+ T cells. FIG. 1C shows stained H&E stained tissue sections. Intestine from Balb/c recipients were sectioned and stained for H&E. Slides were scanned with Leica Aperio VERSA slide scanner and visualized with Aperio ImageScope. FIG. 1D, FIG. 1E, FIG. 1E, FIG. 1F and FIG. 1G show results of analysis of recipient's immune response in tissue by flow cytometry. Balb/c recipients were euthanized at the indicated time points post-hematopoietic stem cell transplantation (HSCT) and tissues were harvested. FIG. 1D is a bar graph showing percent interferon (IFN)-γ in spleen, mesenteric lymph node (mLN) and liver CD4 T cells v. time. FIG. 1E shows number of IFN-γ CD4+ T cells/organ in spleen, mesenteric lymph node (mLN) and liver v. time. Id3-cKO donor CD4+ T cells exhibited normal cytokine-producing capacity represented by the similar percentage (FIG. 1D) and number (FIG. 1E) of IFN-g+ cells in the spleen, mesenteric lymph node (mLN) and liver. FIG. 1F shows a bar graph showing the number and percent IFNγ-secreting intestine-infiltrated donor CD4+ T cells especially those with IFN-g-producing capacity recovered in Balb/c recipients transplanted with wild type (WT) Id3 and with Id3-cKO T cells. FIG. 1G contains bar graphs showing percent CD4+ T cells expressing a4b7, CCR5 and CCR9. *p<0.05, ***p<0.001, student's t-test was used for two-group comparison.

FIG. 2 comprising FIG. 2A, FIG. 2B, FIG. 2C, FIG. 2D, FIG. 2E, FIG. 2F, FIG. 2G, FIG. 2H, FIG. 21, and FIG. 2J shows that Id3 represses genes associated with T cell effector differentiation and inhibitory signaling. Wild type (WT) and Id3-cKO naïve CD4+ T cells were activated and cultured under Th1 polarization conditions. Cells were harvested 24 hr (“TCR primed”) and 96 hr after activation. 96 hr-cultured cells were sorted into CD62L^{hi} and CD62L^{lo} (“Th1 cells”) populations before library preparation. FIG. 2A shows results from Gene Set Enrichment Analysis (GSEA). Hallmark gene set showed significant enrichment of TNF-α signaling via NF-kB, interferon gamma response, IL2-STAT5 signaling, IL6-STAT3 signaling, allograft rejection, interferon alpha response and apoptosis comparing transcriptome from 4-day cultured Id3-cKO Th1-like cells with WT. FIG. 2B show the GSEA determined Id3-regulated gene signature in TCR-primed CD4+ T cells and effector Th1 CD4+ T cells. FIG. 2C, FIG. 2D, FIG. 2E, FIG. 2F, and FIG. 2G show heatmaps that demonstrate 5 categories of differentially expressed genes: FIG. 2C shows transcription factors critical for effector proliferation and differentiation; FIG. 2D shows cytokines that distinguish Th1 from Th2, Th17 cells; FIG. 2E shows 2E cell survival and death molecules; FIG. 2F shows costimulatory molecules important for GVHD T cell proliferation and survival; and FIG. 2G shows inhibitory receptors. FIG. 2H is a flow cytometry analysis and FIG. 21 is a bar graph showing that loss of Id3 leads to increased expression of PD-1 in Th1-like cells. FIG. 2J is a bar graph showing that loss of Id3 leads to increased expression of PD-L1 in Th1-like cells. ***p<0.001.

FIG. 3 comprising FIG. 3A, FIG. 3B, FIG. 3C, FIG. 3D, FIG. 3E, FIG. 3F, FIG. 3G, FIG. 3H, FIG. 3I, and FIG. 3J shows that Id3 deficiency leads to PD-1 upregulation on polyfunctional Th1 cells. FIG. 3A, FIG. 3B, FIG. 3C: Six to eight days after allo-HSCT, cells were isolated from the spleen and liver of Balb/c recipients of donor B6 T cells as described in FIG. 1, and donor T cells were analyzed for effector function. FIG. 3A (flow cytometry plot) and FIG. 3B (bar graph) show the percentage of IFN-gamma+GM-CSF+ polyfunctional cells in spleen and liver. FIG. 3C is a bar graph showing the number of Id3-cKO IFN-gamma⁺GM⁻ CSF⁺ T cells in the spleen and liver. FIG. 3D (flow cytometry plot) and FIG. 3E (bar graph) show the frequency of lamina-propria lymphocyte (LPL) IFNgamma⁺GM-CSF⁺ cells in the intestine of Balb/c recipients 17 days post-HSCT. FIG. 3F, FIG. 3G, and FIG. 3H show the results of gating the IFN-gamma⁺GM-CSF⁺ polyfunctional population for the analysis of inhibitory molecule expression. The bar graphs of FIG. 3G and FIG. 3H show the percentage of PD-1⁺ cells (FIG. 3G) and PD-1 on the surface of individual cells (MFI) (FIG. 3H) on the surface of individual CD4⁺ polyfunctional T cells isolated from both the spleen and the liver of Balb/c recipients 6- and 8-days post-HSCT. FIG. 3I (flow cytometry plot) and FIG. 3J (bar graph) show the percentage of PD-L1-expressing donor CD4+ T cells isolated from the spleen and liver of Balb/c recipients. *p<0.05,***p<0.001, student's t-test.

FIG. 4 comprising FIG. 4A, FIG. 4B, FIG. 4C, FIG. 4D, FIG. 4E, and FIG. 4F shows that PD-1 blockade restored the GVHD-inducing capacity of Id3-cKO T cells. FIG. 4A, FIG. 4B, FIG. 4C, FIG. 4D, FIG. 4E: Balb/b mice were given total body irradiation (TBI, 5 Gy on day −1 and 5 Gy on day 0) followed by infusion of 5×10⁶ B6×B6/SJL F1(CD45.1⁺CD45.2⁺) TCD-BM alone or together with 1×10⁶ B6 (CD45.1-CD45.2⁺) naïve CD4⁺ and 5×10⁵ B6 (CD45.1-CD45.2⁺) naïve CD8⁺ WT or Id3-cKO T cells. Anti-PD-1 antibody (200 g/mouse/injection) was administered intraperitoneally at day 0, 3, 5 and 7 after HSCT. FIG. 4A is a survival curve showing survival rates of Balb/b mice. FIG. 4B contains bar graps for spleen, liver and intestind showing donor CD4 T cell number.

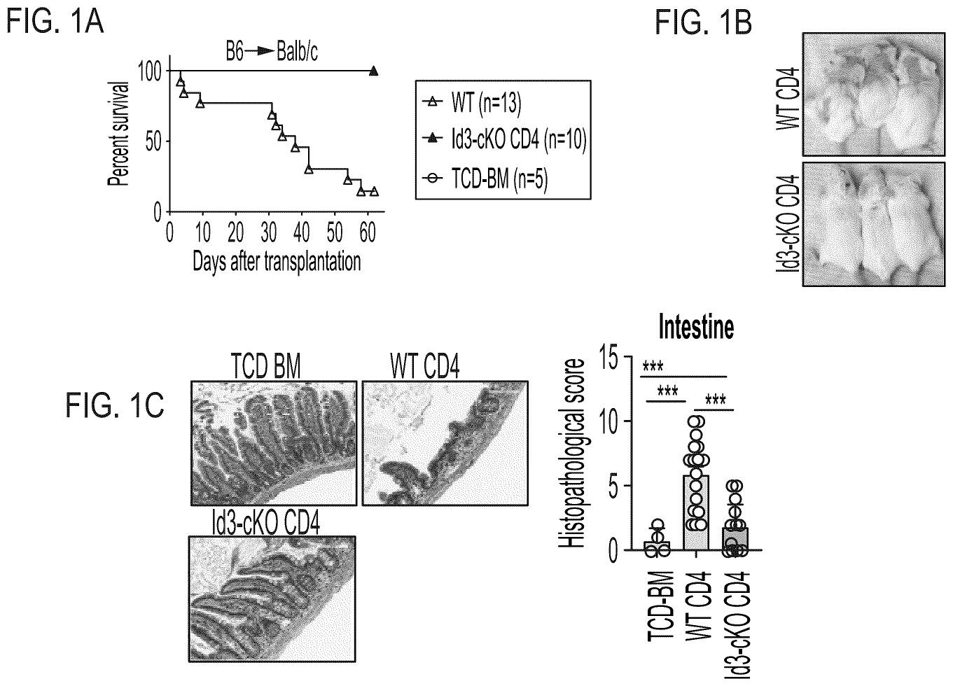

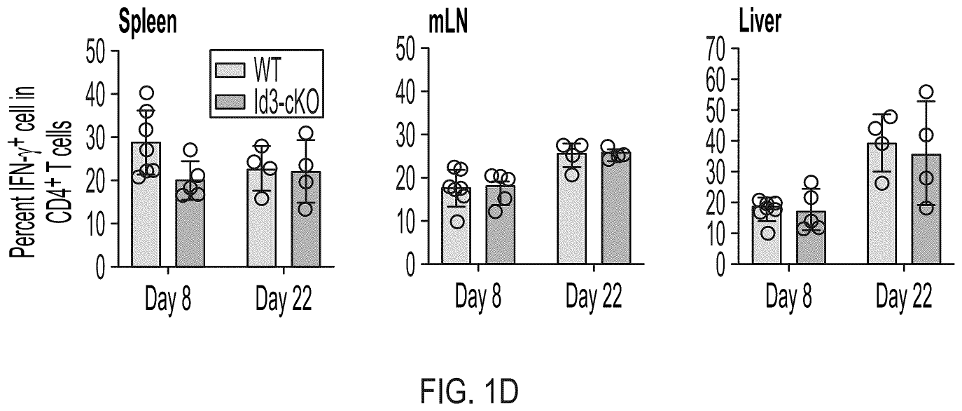

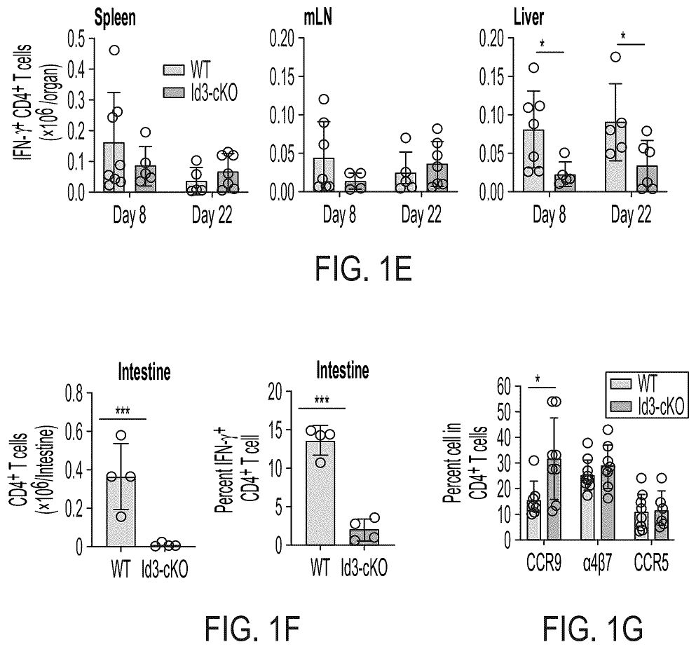

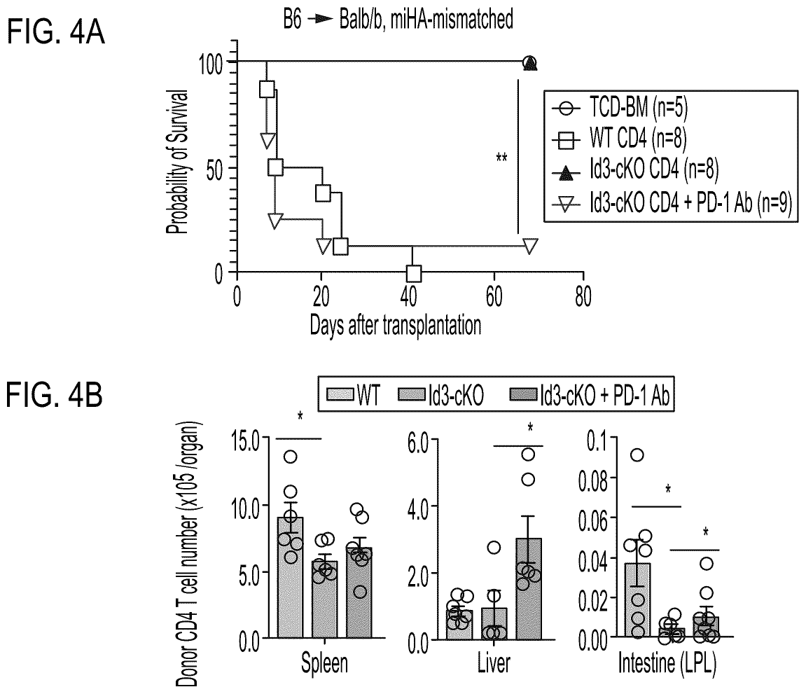

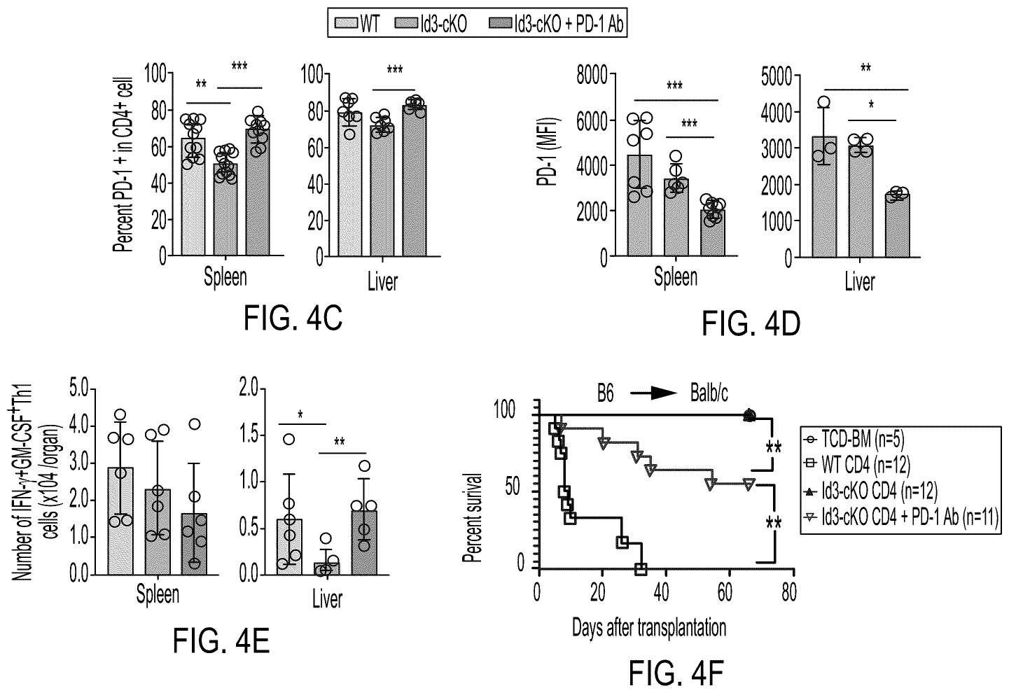

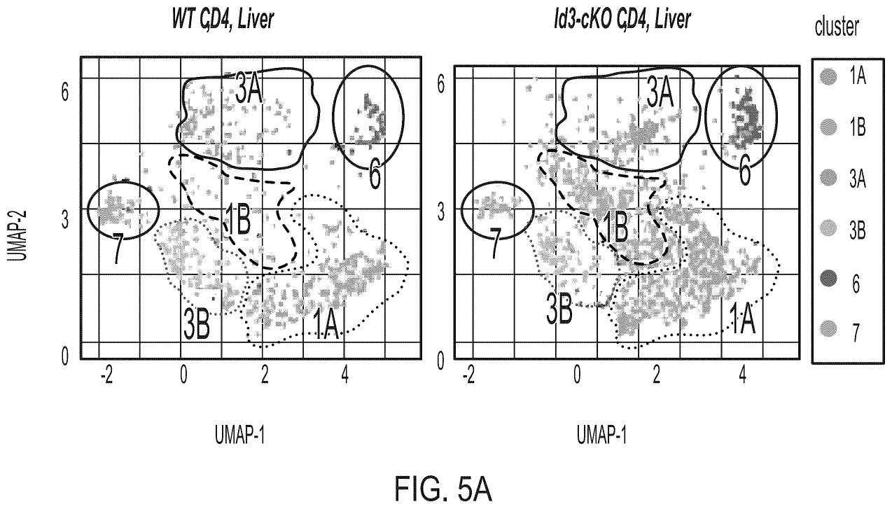

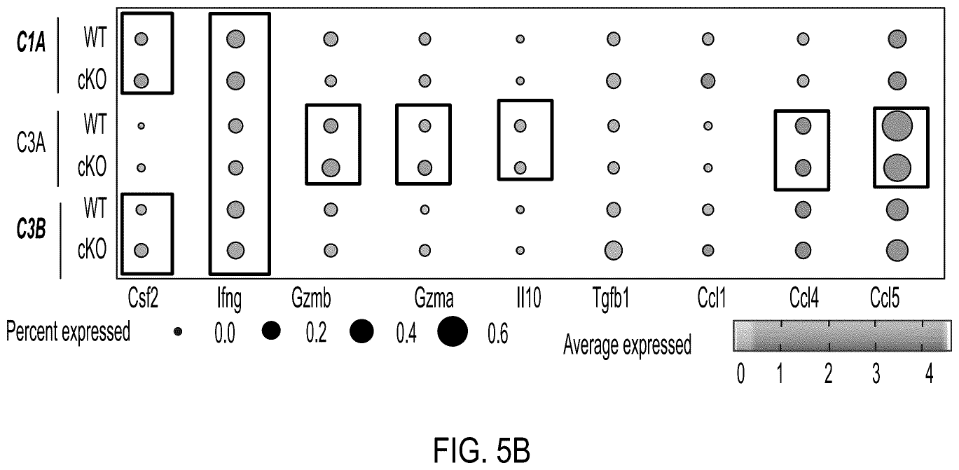

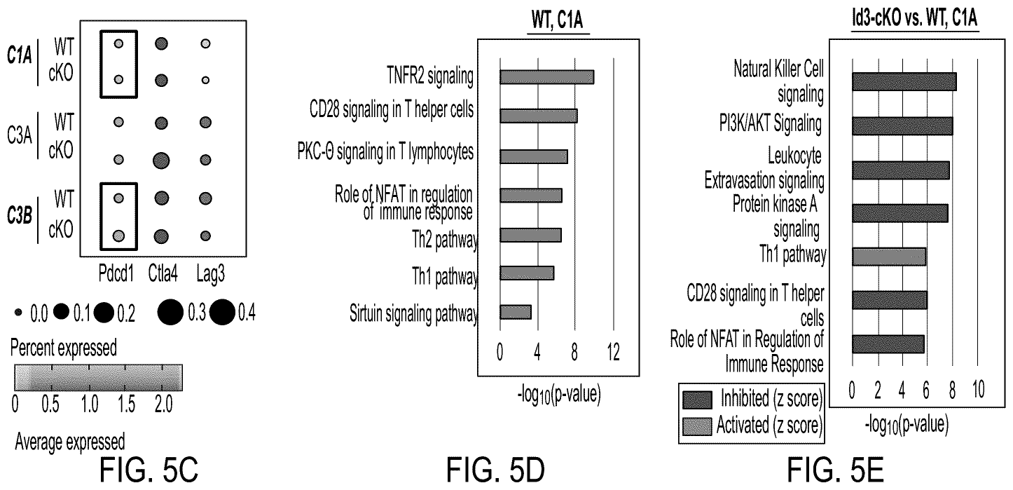

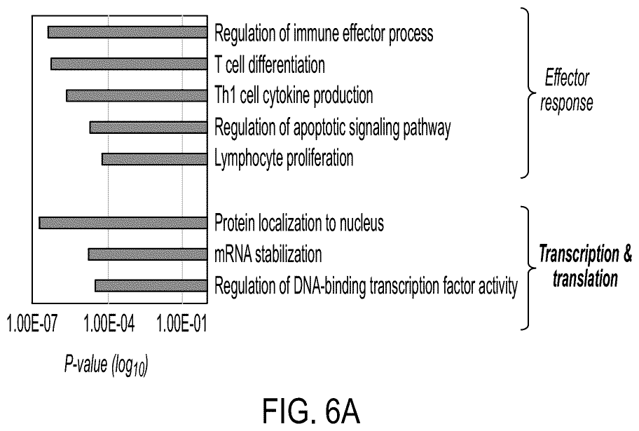

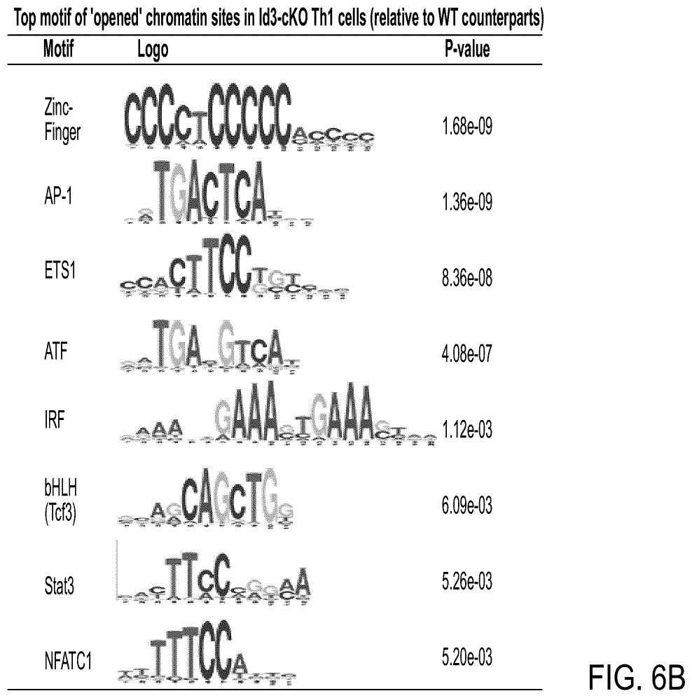

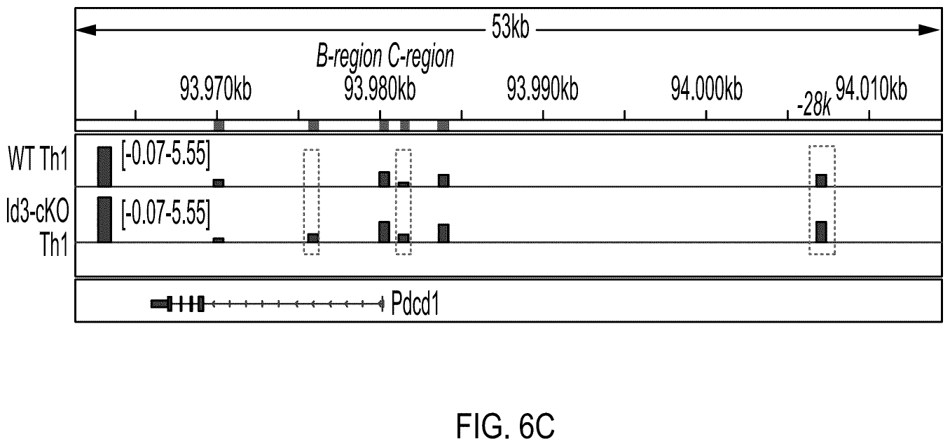

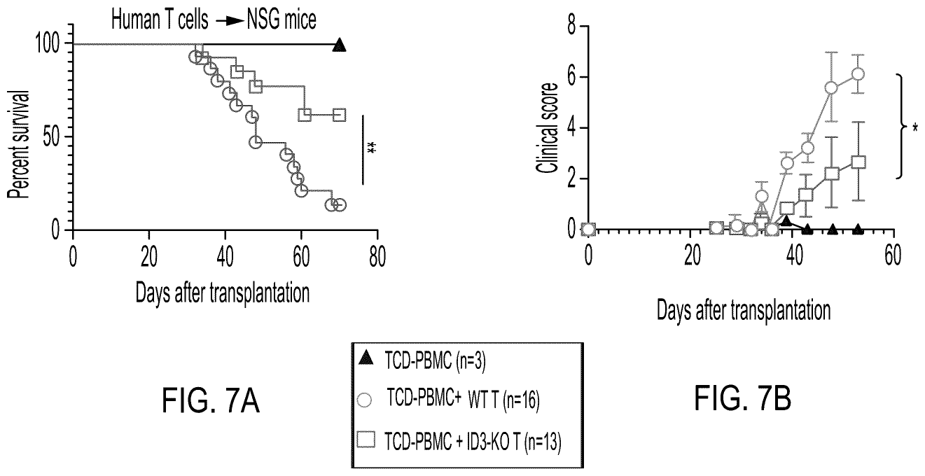

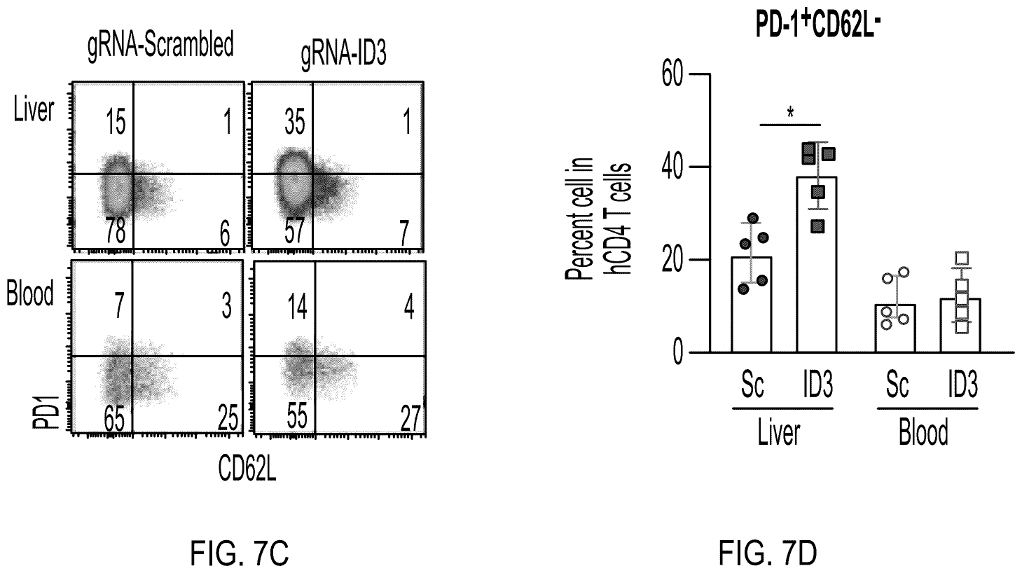

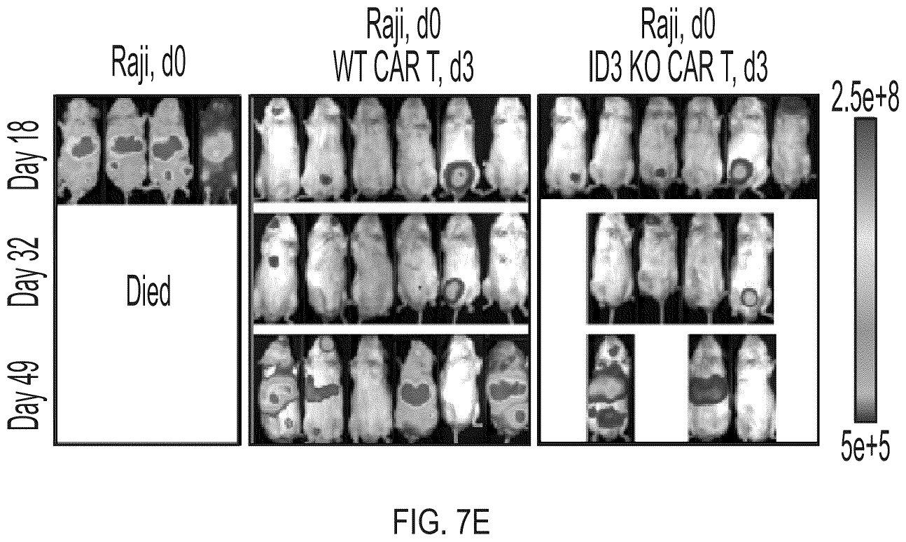

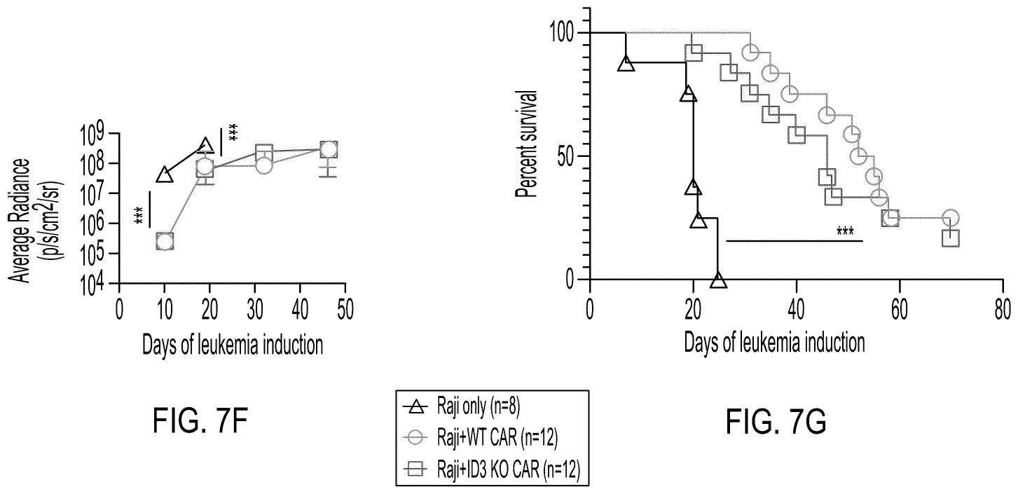

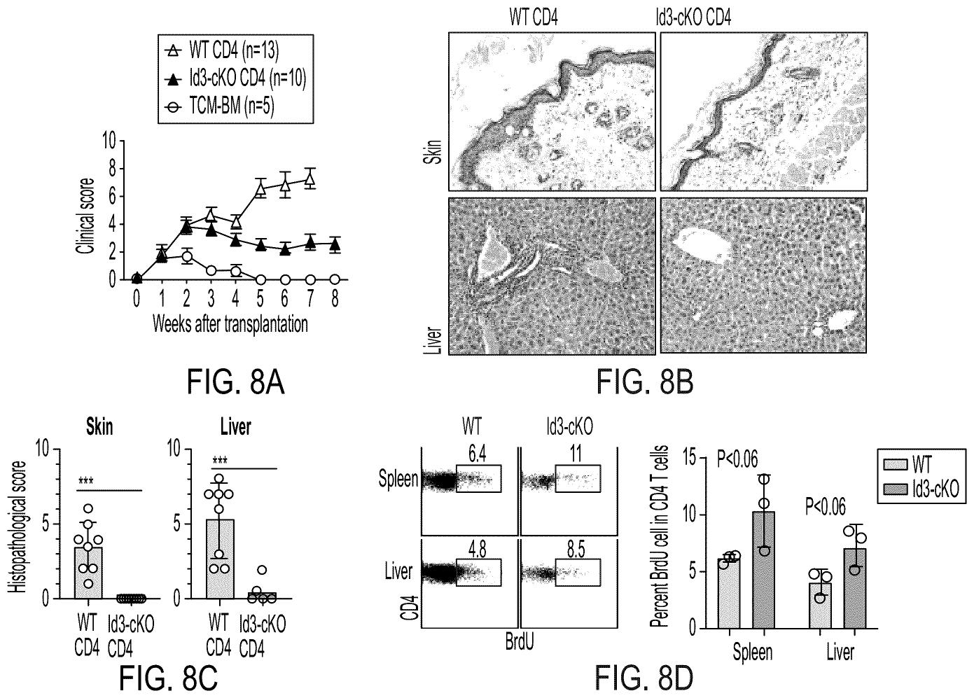

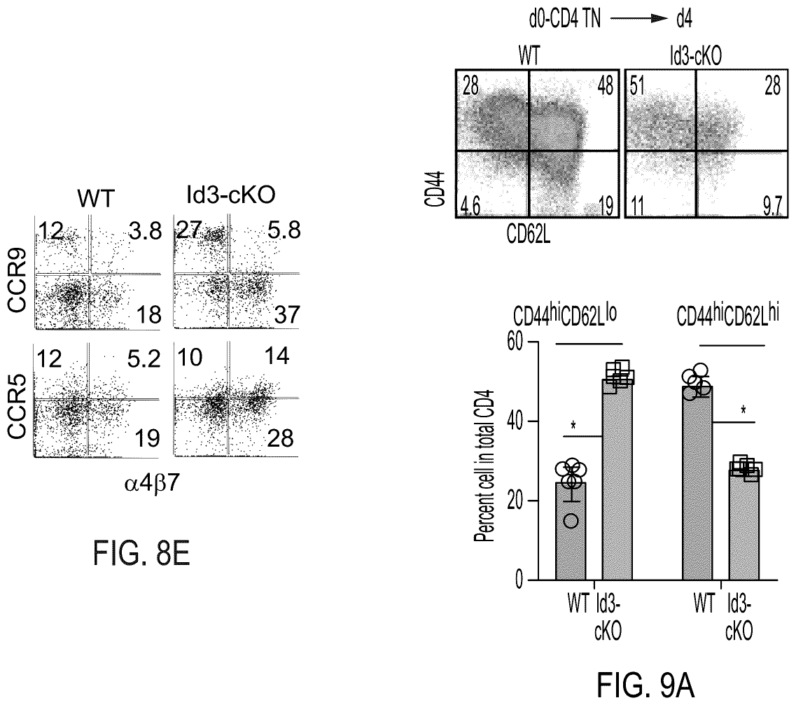

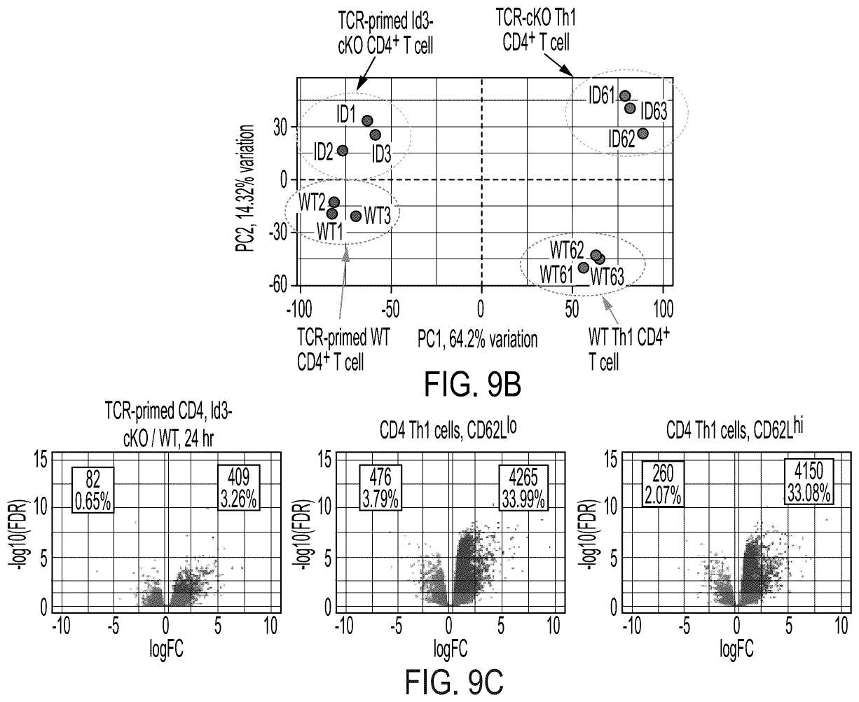

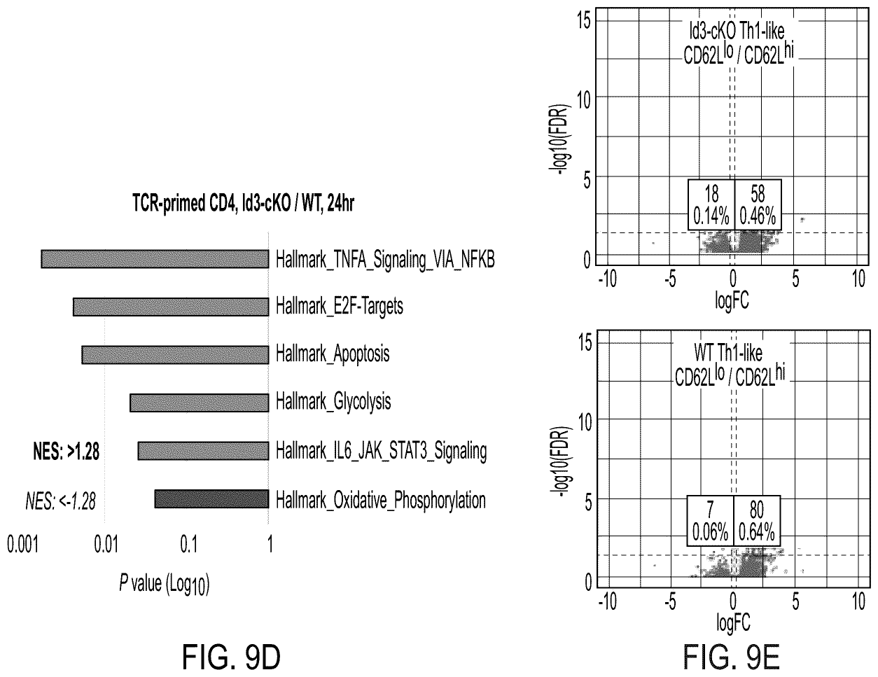

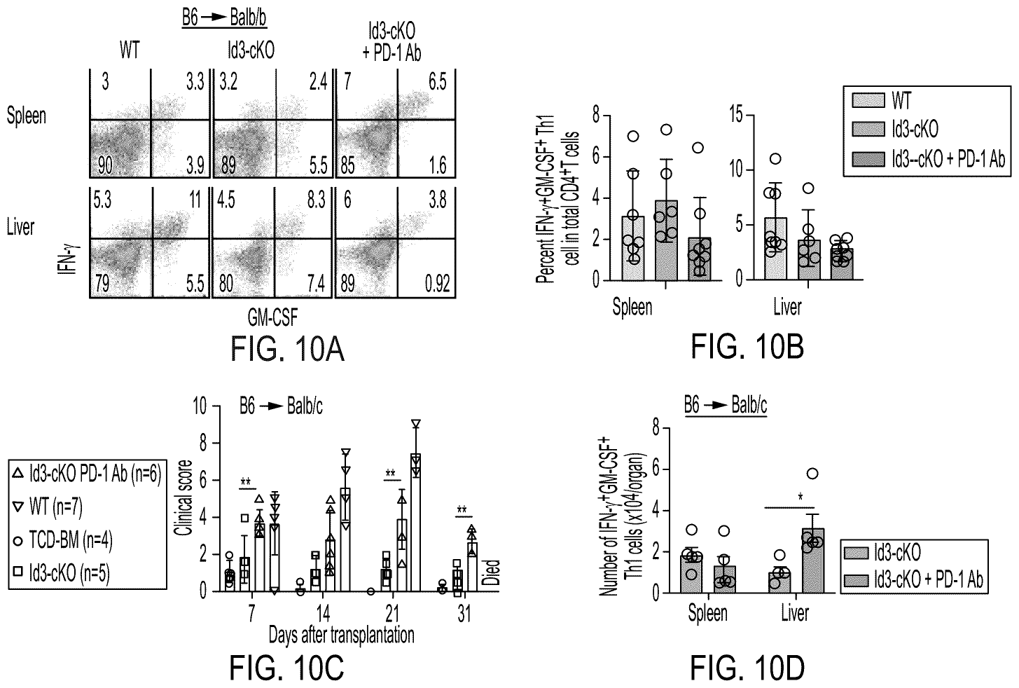

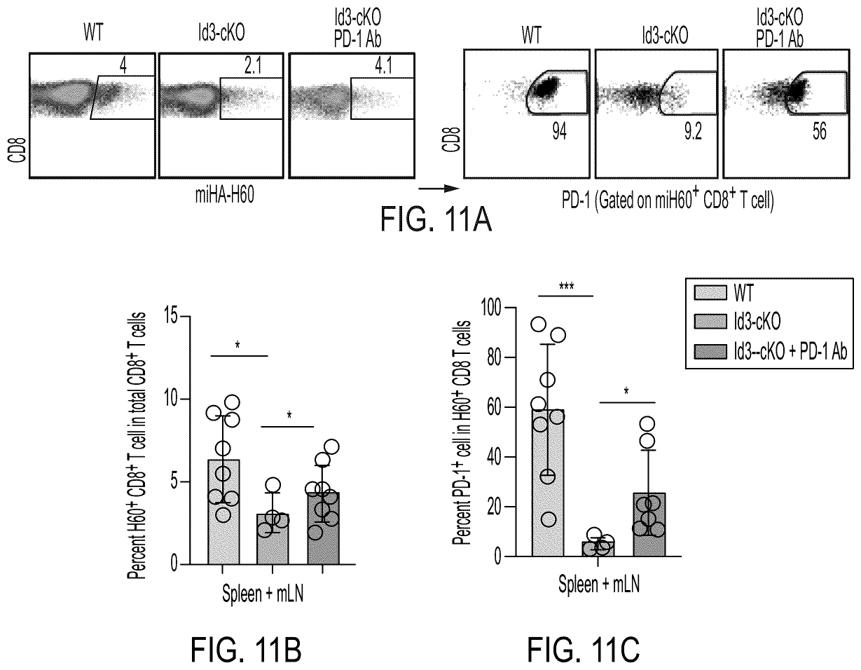

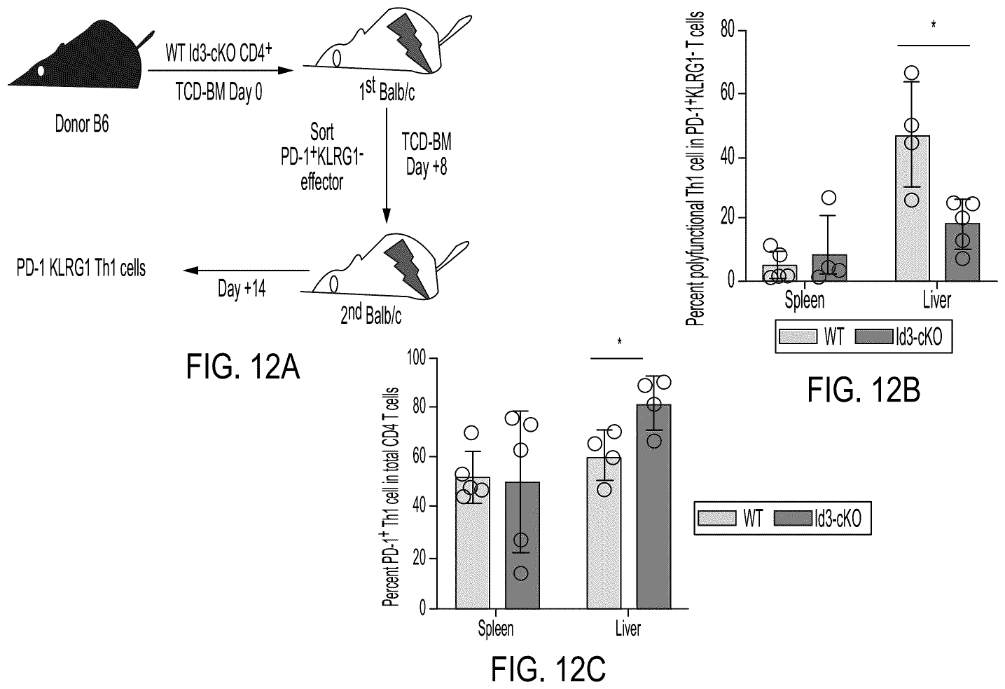

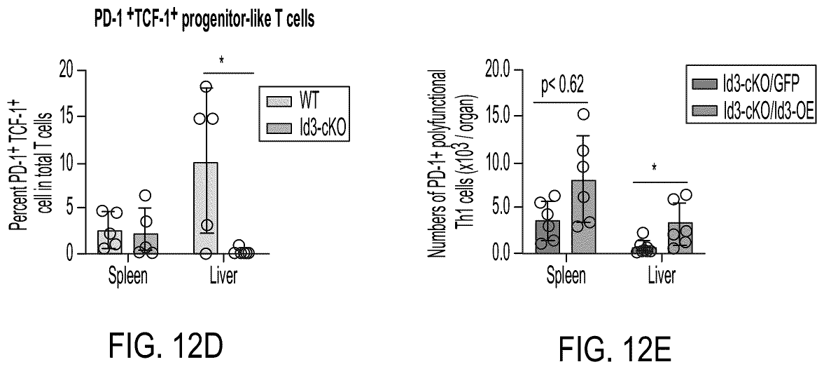

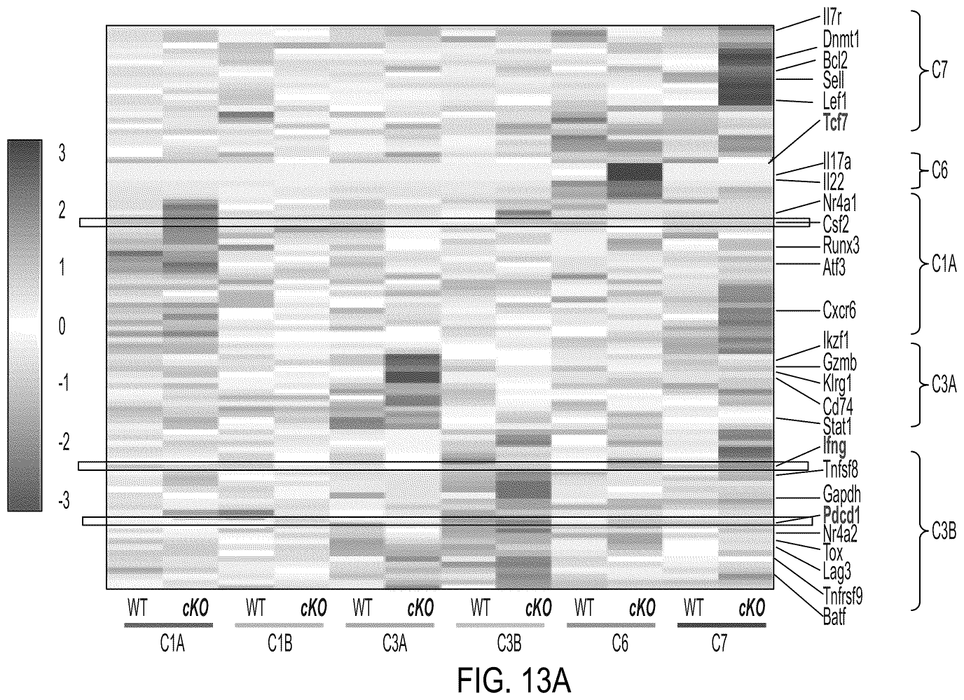

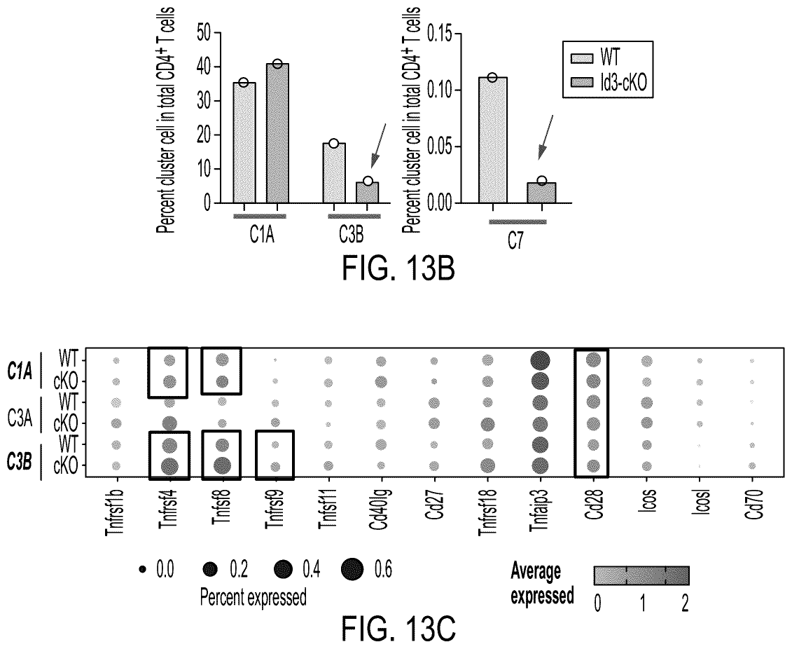

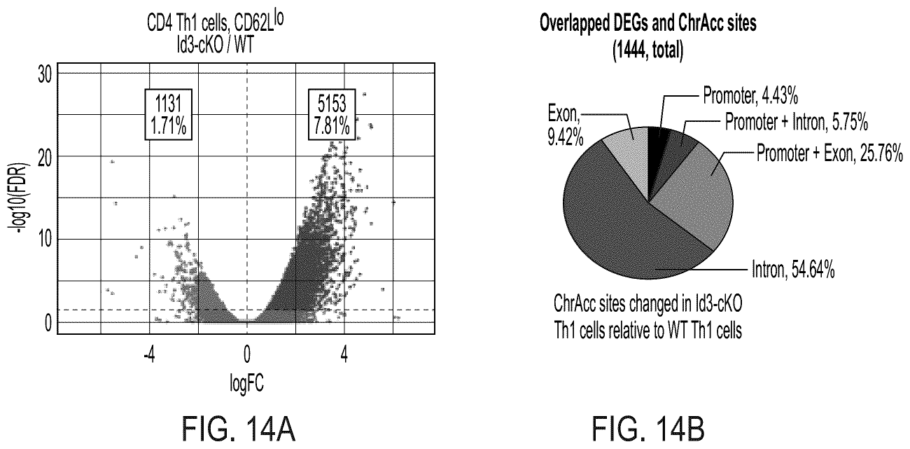

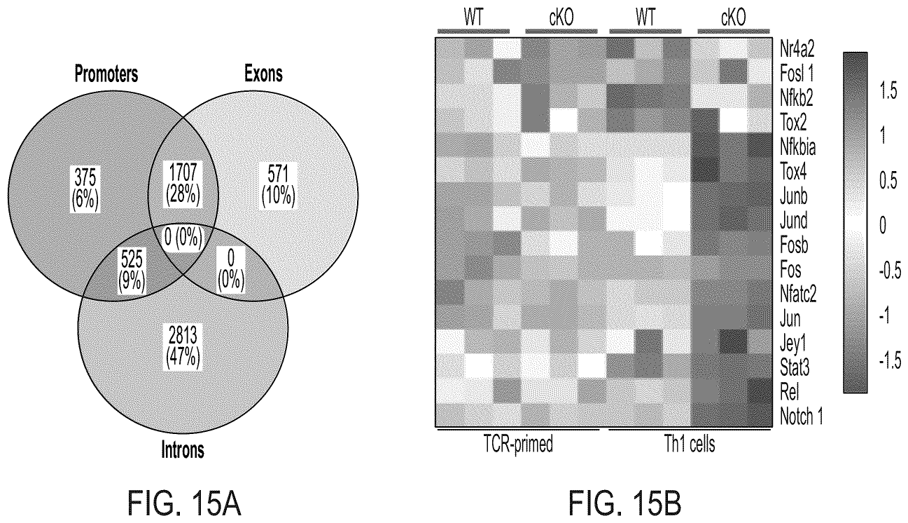

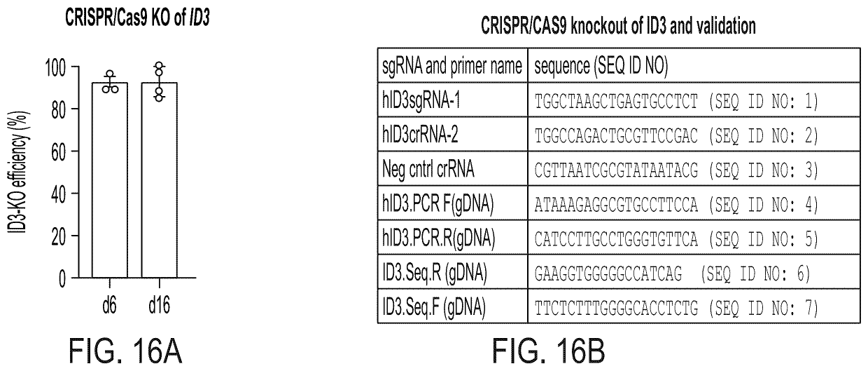

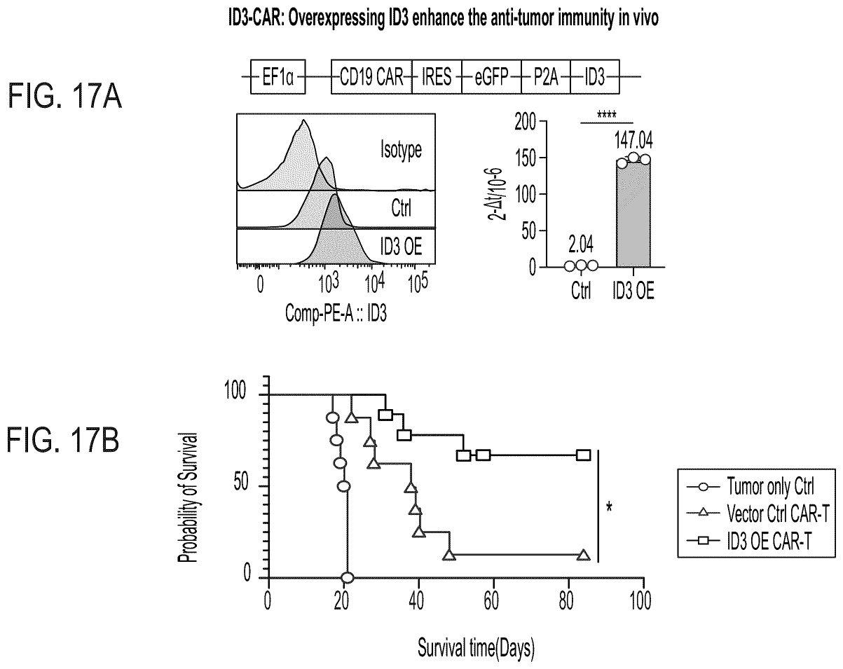

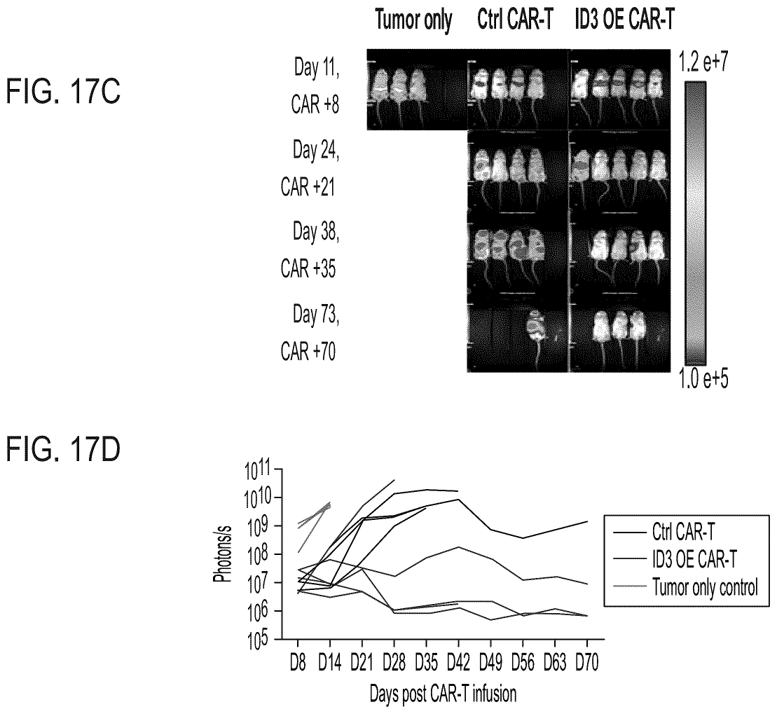

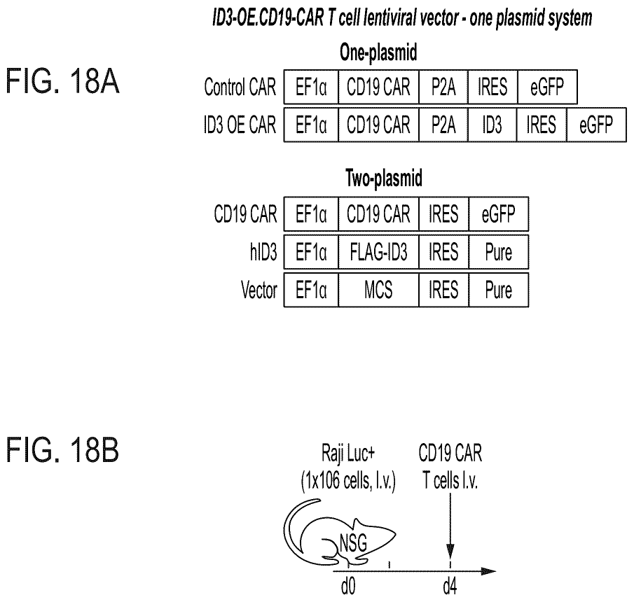

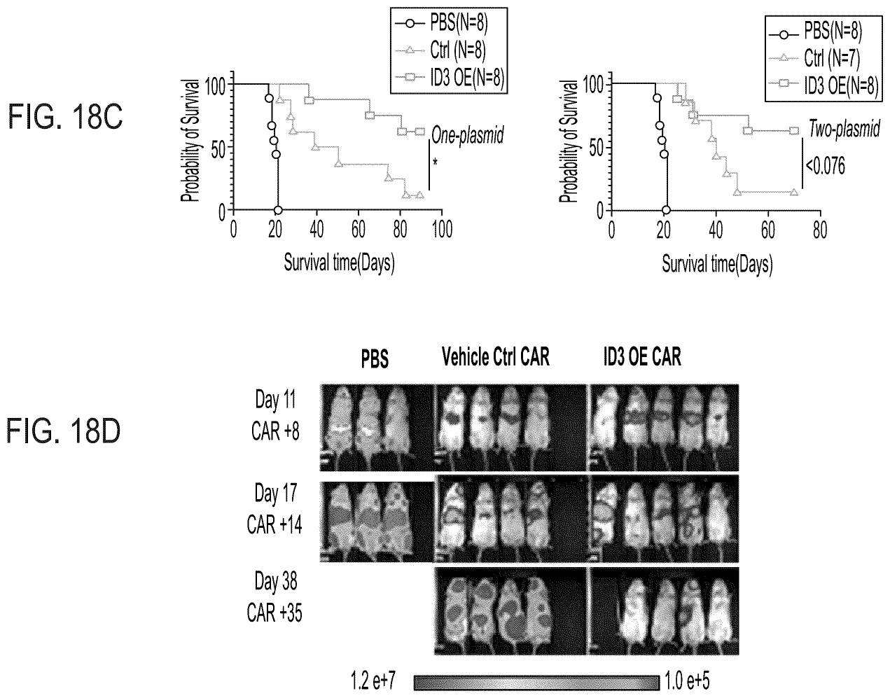

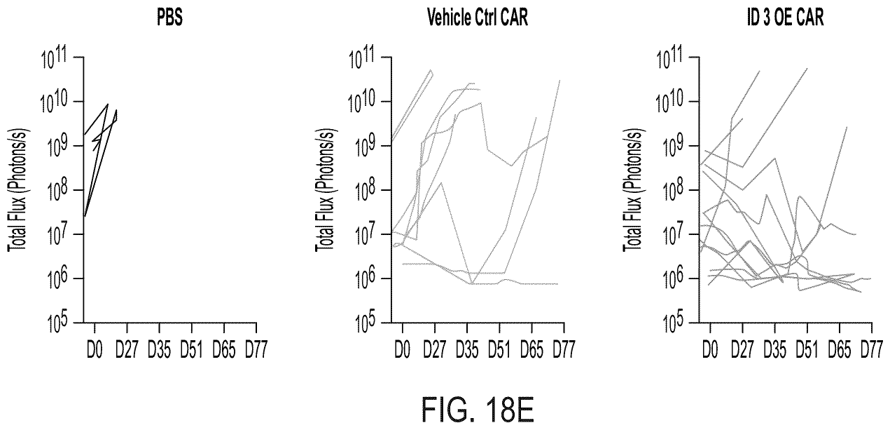

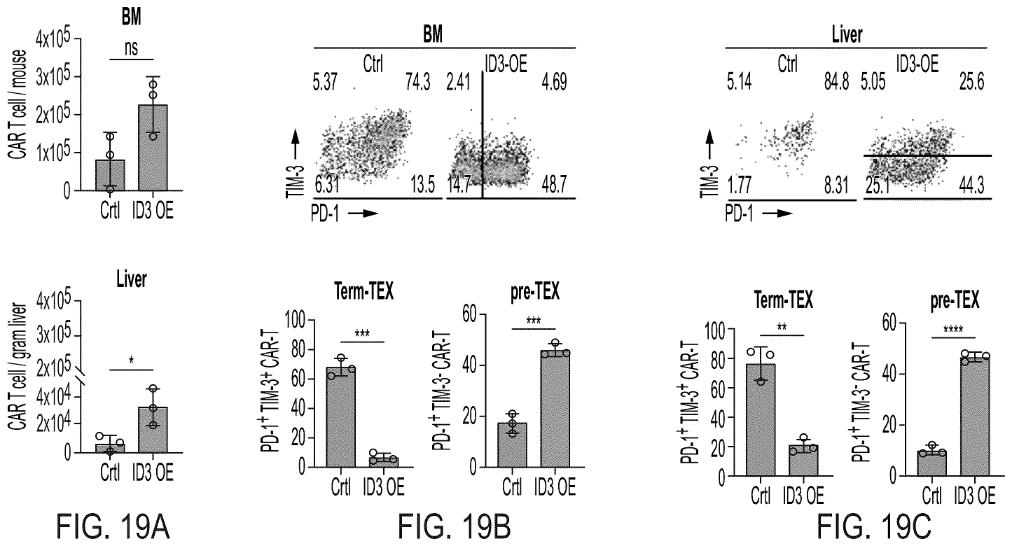

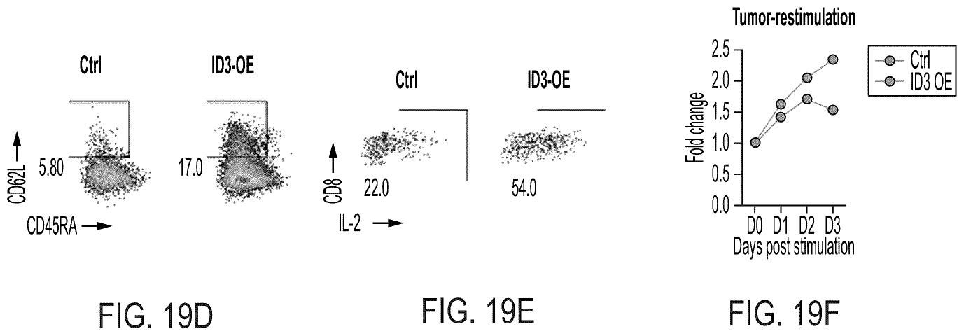

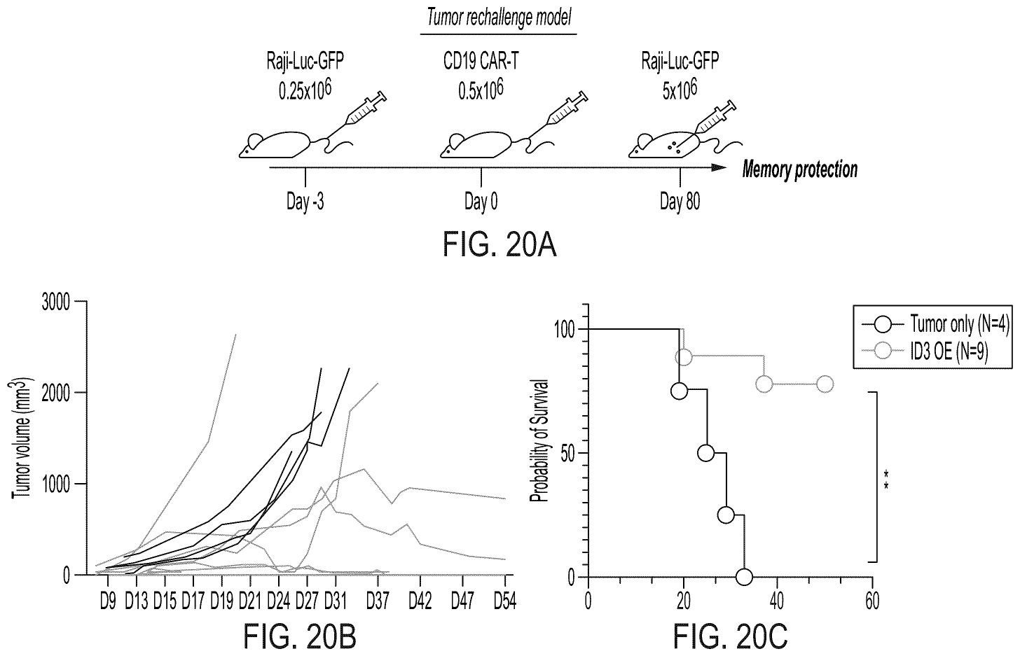

Seventeen days post-HSCT, cells were isolated from the spleen, liver and intestine of recipients, enumerated and stained for flow cytometry analysis. The bar graphs in FIG. 4C show the percentage of PD-1⁺ donor CD4⁺ T cells in the spleen and liver. The bar graphs in FIG. 4D show PD-1 protein levels on the surface of donor CD4⁺ T cells isolated from the spleen and liver of Balb/b recipients. The bar graphs in FIG. 4E show the number of IFN-gamma+GM-CSF+ population in the spleen and liver. The survival curve in FIG. 4F shows the survival rate. Balb/c transplantation was performed as described in FIG. 1. Anti-PD1 antibody was administered following the same regimen as in Balb/b recipients. *p<0.05, **p<0.01, ***p<0.001, student's t-test.

FIG. 5 comprising FIG. 5A, FIG. 5B, FIG. 5C, FIG. 5D and FIG. 5E shows characterization of liver infiltrated donor CD4 T cells using scRNA-seq. Allo-HSCT was performed as described in FIG. 1. Three weeks after HSCT, livers were harvested from recipients for T cell enrichment. FIG. 5A is a UMAP dimensional reduction analysis of single cell transcriptome from liver infiltrated WT and Id3-cKO donor CD4⁺ T cells identified 6 clusters: 1A, 1B, 3A, 3B, 6, 7. FIG. 5B shows expression of effector molecules in WT and Id3-cKO CD4⁺ T cells in clusters 1A, 3A and 3B. The size of the balls represents percentage of cells expressing the molecule in that cluster, while the shades of red represent the average level of the molecule in the cluster. FIG. 5C shows expression of inhibitory molecules in WT and Id3-cKO CD4⁺ T cells in clusters 1A, 3A and 3B. FIG. 5D shows characterization of WT cluster 1A using Ingenuity Pathway Analysis (IPA) of differential expression of genes (DEGs) in 1A compared to the average transcriptome of other clusters. FIG. 5E shows IPA characterization of Id3-cKO cluster 1A relative to WT cluster 1A.

FIG. 6 comprising FIG. 6A, FIG. 6B, and FIG. 6C shows that Id3 restricts chromatin accessibility at specific gene loci. Naïve CD4⁺ T cells were cultured under Th1 polarization conditions for 4 days. CD62^{lo} effector cell population were sorted for ATAC-seq and RNA-seq libraries preparation. FIG. 6A shows differential chromosome accessibility (ChrAcc) sites were assigned to genes less than 50 kb upstream and downstream. Differential expression of genes (DEGs) associated with opened chromatins in Id3-cKO Th1 cells were subject to GO term analysis. FIG. 6B shows motif analysis of opened chromatin sites in Id3-cKO Th1 cells. FIG. 6C shows differential accessible windows along the gene pdcdl regulatory loci.

FIG. 7 comprising FIG. 7A, FIG. 7B, FIG. 7C, FIG. 7D, FIG. 7E, FIG. 7F, and FIG. 7G shows that ID3 ablation in human T cells reduces xeno-GVHD progression while preserving anti-leukemia activity. ID3 was knocked out from human T cells (including both CD4⁺ T and CD8⁺ T cells) using CRISPR-Cas9 technology and maintained in culture for 7-9 days. NSG mice were intravenously infused with TCD-PBMCs (10×10⁶/mouse) alone or together with WT or ID3-CRISPR-KO human CD4⁺ T cells (10×10⁶ cells/mouse) and CD8⁺ T cells (10×10⁶ cells/mouse). FIG. 7A shows survival rates of NSG mice receiving WT and ID3-CRISPR-KO human T cells. FIG. 7B shows clinical scores of NSG mice receiving WT and ID3-CRISPR-KO human T cells. FIG. 7C (flow cytometry plots) and FIG. 7D (bar graphs) show the presence of PD-1⁺ T cells in both the liver and circulating peripheral blood. FIG. 7E, FIG. 7F, FIG. 7G: Firefly Luciferase-eGFP-expressing Raji cells (0.2×10⁶) were injected to NSG mice on day −3. WT or ID3-CRISPR-KO CD19-CAR-T cells (2.5×10⁶ cells/mouse) were infused on day 0. FIG. 7E shows results of IVIS in vivo imaging showing leukemia growth and the comparable tumor control potency by WT and ID3-CRISPR-KO CD19-CAR T cells as demonstrated in FIG. 7F. FIG. 7G is a survival curve showing the overall survival of leukemia-bearing mice treated with or without WT or ID3-CRISPR-KO CD19-CAR T cells (n=8-12). *p<0.05, **p<0.01,***p<0.001.

FIG. 8 comprising FIG. 8A, FIG. 8B, FIG. 8C, FIG. 8D, and FIG. 8E shows that Id3-cKO T cells fail to mediate GVHD in mice. Lethally irradiated Balb/c mice were infused with B6×B6/SJL F1 mouse TCD-BM alone or together with WT and Id3-cKO B6 naïve CD4⁺ T cells as described in FIG. 1. FIG. 8A shows the clinical score of Balb/c recipients. Each curve represents the average clinical scores from all mice in that group. FIG. 8B are skin and liver tissue sections from Balb/c recipients. Skin and liver tissue were sectioned and stained for H&E. Slides were scanned with Leica Aperio VERSA slide scanner and visualized with Aperio ImageScope. FIG. 8C shows bar graphs showing the average histopathological score of recipients from the same group (mean±SD). FIG. 8D shows flow cytometry plots and bar graphs showing show the percentage of BrdU⁺ donor CD4⁺ T cells. Donor cells were isolated from the spleen of liver of Balb/c recipients 4 hours after pulsing with BrdU at day 13 after transplantation, stained and analyzed with flow cytometer. FIG. 8E shows flow cytometry plots showing the expression of chemokine receptors CCR5, CCR9 and integrin α4β7. ***p<0.001, student's t-test.

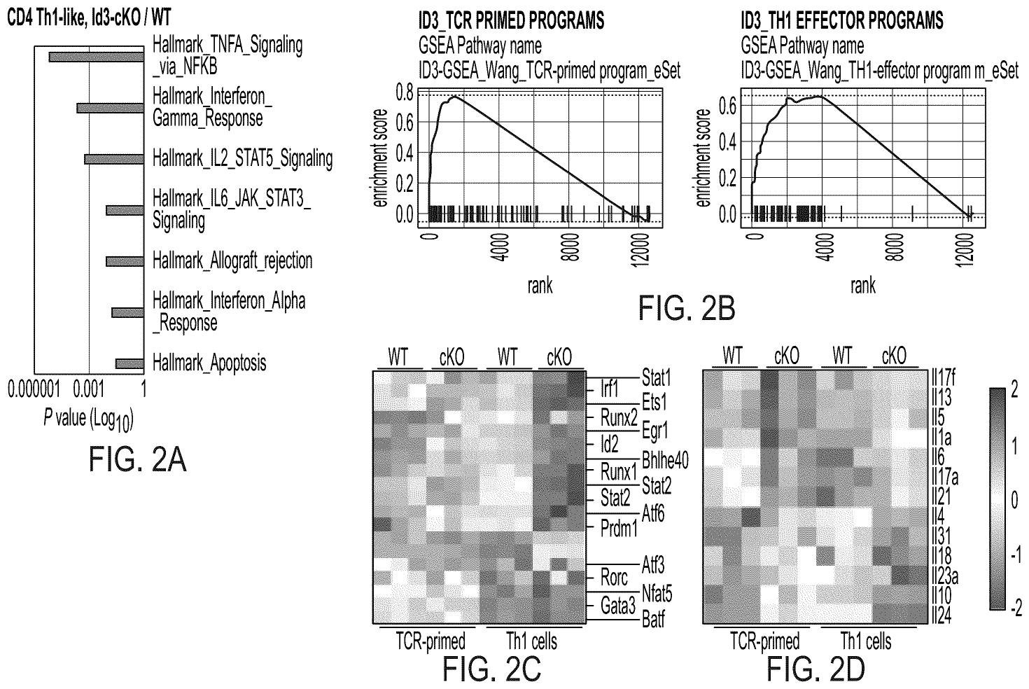

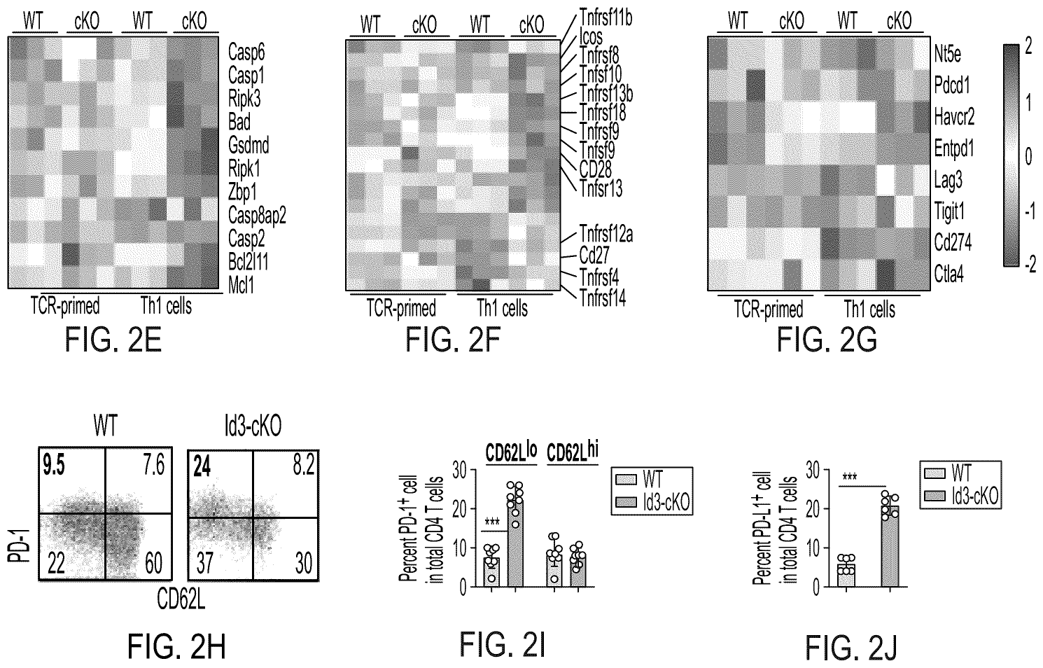

FIG. 9 comprising FIG. 9A, FIG. 9B, FIG. 9C, FIG. 9D, and FIG. 9E shows RNA-seq analysis of WT and Id3-cKO T cells. Naïve CD4⁺ T cells were cultured as described in FIG. 2. FIG. 9A shows flow cytometry plots and bar graphs showing the percentage of CD62L^{lo}CD44^{hi} and CD62L^{hi}CD44^{hi} populations. FIG. 9B shows the results of Principal Component Analysis (PCA) dimension reduction analysis, which showed distribution of 24 hr-cultured TCR-primed WT and Id3-cKO CD4 T cells and 96 hr-cultured CD62L^{lo}CD44^{hi} WT and Id3-cKO Th1 cells. Differential expression of genes (DEGs) was defined by fold change, >2-fold; p-value <0.05. FIG. 9C shows volcano plots showing DEGs comparing TCR-primed Id3-cKO with WT cells (left panel), CD62L^{lo}CD44^{hi} Id3-cKO with WT cells (middle panel), and CD62L^{hi}CD44^{hi} Id3-cKO with WT cells (right panel). FIG. 9D shows results of GSEA querying genes up- or down-regulated in TCR stimulated Id3-cKO cells compared to WT showed significant enrichment of gene sets under the Hallmark category. FIG. 9E shows volcano plots of the DEGs between the CD62L^{lo} and CD62L^{hi} populations of either Id3-cKO cells or WT Th1 cells. *p<0.05, student's t-test.

FIG. 10 comprising FIG. 10A, FIG. 10B, FIG. 10C and FIG. 10D shows that PD-1 antibody treatment rescues donor CD4⁺ T cells in MHCI-mismatched murine GVHD model. Lethally irradiated Balb/b mice were infused with B6×B6/SJL F1 mouse TCD-BM alone or together with WT and Id3-cKO B6 naïve CD4⁺ T cells as described in FIG. 4A-E. Anti-PD-1 antibody (200 g/mouse/injection) was administered intraperitoneally at day 0, 3, 5 and 7 after HSCT. FIG. 10A show flow charts showing the frequency of IFN-gamma and GM-CSF. Cells were isolated from the spleen and liver of Balb/b recipients to evaluate cytokine-producing capacity of donor CD4⁺ T cells. FIG. 10B show bar graphs of the percentage of IFN-gamma+GM-CSF+ population in the spleen and liver. FIG. 10C shows time kinetic monitoring of clinical scores indicative of GVHD progression in Balb/c recipients treated with solvent or anti-PD-1 antibody. FIG. 10D is a bar graph showing the number of IFN-gamma+GM-CSF+Id3-cKO Th1 cells in the spleen and liver of Balb/c mice. *p<0.05, ** p<0.01, student's t-test.

FIG. 11 comprising FIG. 11A, FIGS. 11B, and 11C, shows that treatment with a PD-1 antibody (BioXCell, New Hampshire) rescues alloreactive Id3-cKO H60⁺CD8⁺ T cells. Lethally irradiated Balb/b mice were infused with B6×B6/SJL F1 mouse TCD-BM alone or together with WT and Id3-cKO B6 naïve CD4⁺ T cells as described in FIG. 4A-E. Cells were isolated from the spleen and mLN of Balb/b recipients 17 after transplantation. FIG. 11A are flow cytometry plots showing the frequency of H60⁺CD8⁺ T cells and their expression of PD-1. FIG. 11B are bar graphs showing the percentage of H60⁺CD8⁺ T cells in total CD8 donor T cells. FIG. 11C are bar graphs showing the percentage of PD-1+ population in H60⁺CD8 T cells. *p<0.05, ***p<0.001, student's t-test.

FIG. 12 comprising FIG. 12A, FIG. 12B, FIG. 12C, FIG. 12D and FIG. 12E shows that Id3 maintains polyfunctional Th1 cells and PD-1⁺TCF-1⁺ progenitor-like T cells that infiltrate the liver. FIG. 12A is a schematic description of experimental procedure. Briefly, lethally irradiated primary (1^{st}) Balb/c mice were infused with B6×B6/SJL F1 mouse TCD-BM alone or together with WT and Id3-cKO B6 naïve CD4⁺ T cells as described in FIG. 1. Eight days later, donor (CD45.1-CD45.2⁺) CD4⁺ T cells were isolated from these 1^{st} Balb/c mice, followed by sorting PD-1⁺KLRG⁻ CD4⁺ T cells. Purified alloreactive PD-1⁺KLRG⁻CD4⁺ T cells were mixed with B6×B6/SJL F1(CD45.1⁺CD45.2⁺) TCD-BM and infused to lethally irradiated secondary Balb/c mice (2^{nd}). Fourteen days after the adoptive transfer, spleen and liver were harvested from these 2^{nd} recipients to analyze donor CD4 T cells FIG. 12B, FIG. 12C, and FIG. 12D are bar graphs. FIG. 12B bar graphs show the percentage of IFN-gamma+GM-CSF+ polyfunctional population in PD-1⁺KLRG1⁻ donor CD4⁺ T cells derived from the spleen and liver. FIG. 12C bar graphs show the percentage of PD-1⁺Th1 in total donor CD4⁺ T cells in the spleen and liver of 2^{nd} recipients. FIG. 12D bar graphs show percentage of PD-1⁺TCF-1⁺ progenitor-like T cells in total donor CD4⁺ T cells in the spleen and liver. FIG. 12E bar graph shows the number of PD-1⁺ cells in IFN-gamma ⁺GM-CSF⁺ polyfunctional Th1 cells in the spleen and liver of Balb/c recipients. Id3 ectopic expression B6 mice were generated by reconstitution of lethally irradiated B6 mice with CD4-Cre*Id3flf c-kit cells infected with GFP-expressing virus (Id3-cKO/GFP) or Id3 ectopic expression virus (Id3-cKO/Id3-OE). Three months after reconstitution, Id3-cKO/GFP and Id3-cKO/Id3-OE CD4⁺ T cells (0.1×10⁶ cells/mouse) were isolated from these B6 mice, mixed with B6×B6/SJL F1 mouse TCD-BM cells (5×10⁶ cells/mouse) and infused into lethally irradiated Balb/c mice. Eight days later, donor (CD45.1-CD45.2⁺) CD4⁺ T cells were isolated from these Balb/c mice to test the presence of CD4⁺ polyfunctional Th1 cells. *p<0.05, student's t-test.

FIG. 13 comprising FIG. 13A, FIG. 13B, and FIG. 13C shows scRNA-seq data from liver-infiltrated donor CD4⁺ T cells. FIG. 13A is a heatmap of all differential expression of genes (DEGs) in WT and Id3-cKO donor CD4 T cells in clusters 1A, 3A, 3B, 6 and 7. FIG. 13B shows bar graphs of the percentage of WT and Id3-cKO donor CD4 T cells in clusters 1A, 3B and 7. FIG. 13C shows the expression pattern of cell surface costimulatory and coinhibitory molecules in WT and Id3-cKO cells in clusters 1A, 3A and 3B.

FIG. 14 comprising FIG. 14A and FIG. 14B shows differential chromosome accessibility (ChrAcc) sites in cultured Th1 cells analyzed by the assay for transposase-accessible chromatin with sequencing (ATAC-seq). FIG. 14A shows ATAC-seq analysis was performed as described in FIG. 6. Numbers and percentages of ChrAcc sites with enhanced (red) or decreased (blue) accessibility in CD62L^{lo} population of Id3-cKO Th1 cells compared to WT. FIG. 14B shows enhanced ChrAcc sites overlapped with differential expression of genes (DEGs) in Th1 cells were extracted. Pie chart demonstrates the distribution of these differential ChrAcc sites along the regulator regions in the DEGs.

FIG. 15 comprising FIG. 15A and FIG. 15B shows differential ChrAcc sites in cultured Th1 cells analyzed by ATAC-seq and DEGs identified by bulk-RNA-seq analysis. FIG. 15A is a Venn diagram depicting the distribution of those enhanced ChrAcc sites in the promoters, exons and introns comparing Id3-cKO with WT Th1 cells. FIG. 15B is a heatmap that depicts the expression of cell fate-determining TFs in TCR-primed CD4⁺ T cells and WT and Id3-cKO Th1 cells. RNA-seq analysis is described in FIG. 2.

FIG. 16 comprising FIG. 16A and FIG. 16B shows sequence analysis of ID3-KO in human T cells. CRISPR/Cas9 KO of ID3 in human T cells is described in FIG. 7. FIG. 16A: Clonal sequencing analysis shows the efficiency with gRNA-mediated CRISPR-knockout of ID3. FIG. 16B provides the sequences used for sgRNAs and primers. SEQ ID NO: 1: tggctaagctgagtgcctct; SEQ ID NO: 2: tggccagactgcgttccgac; SEQ ID NO: 3: cgttaatcgcgtataatacg; SEQ ID NO: 4: ataaagaggcgtgccttcca; SEQ ID NO: 5: catccttgcctgggtgttca; SEQ ID NO: 6: gaaggtgggggccatcag; SEQ ID NO: 7: ttctctttggggcacctctg.

FIG. 17 comprising FIG. 17A, FIG. 17B, FIG. 17C, and FIG. 17D shows that overexpressing ID3 enhances the anti-tumor effect of CAR-T cells in vivo. FIG. 17A is a schematic showing the vector structure encoding CAR and Id3. The expression of ID3 was verified using real-time RT-PCR and flow cytometric analysis. FIG. 17B, FIG. 17C, FIG. 17D: FIG. 17B shows survival curves for leukemia-bearing NSG mice treated with or without CAR-T cells. Survival curves are plotted as probability of survival (y-axis) versus survival time (days) (x-axis) for tumor only control (red); vector control CAR-T (green) and Id3 overexpression in CAR-T cells (blue). FIG. 17C shows enhanced anti-tumor immunity of CAR-T overexpressing ID3 in vivo at day 11, day 24, day 38, and day 73. 0.25×10⁶ Raji-Luc-eGFP cells were inoculated through tail vein injection at day 0. On day 3, 0.5×10⁶ unstimulated WT or Id3^{OE} anti-CD19 CAR-T cells were injected through the tail vein. Controls are tumor only and CAR-T control. FIG. 17D shows the dynamic change of the total photons of each mouse. Graph is a plot of photonesis (y axis) versus days post CAR-T infusion. Controls are tumor only and CAR-T control.

FIG. 18 comprising FIG. 18A, FIG. 18B, FIG. 18C, FIG. 18D and FIG. 18E shows that ID3^{OE} in BBz-CAR T cells drastically augments their anti-tumor activity. We transduced human T cells (CD4⁺ and CD8⁺) with a lentivirus encoding CD19-4-1BB-CAR (BBz CAR), together with a lentivirus encoding ID3 or GFP, to produce ID3^{OE}-BBz-CAR T cells or Con-BBz-CAR T cells, respectively (FIG. 18A, one plasmid system). FIG. 18A shows plasmid construction for a one plasmid system embodiment and for a two plasmid system embodiment. FIG. 18 B is a schematic showing the CAR-T cell treatment regimen. FIG. 18C is a survival curve showing survival of human xenograft leukemia NSG mice. FIG. 18D shows results of IVIS in vivo imaging showing leukemia growth. FIG. 18E are graphs showing tumor growth in the presence of PBS; a vehicle control cAR, and ID3^{OE}-CAR. *, P<0.05.

FIG. 19 comprising FIG. 19A, FIG. 19B, FIG. 19C, FIG. 19D, FIG. 19E, and FIG. 19F shows that human ID3^{OE} CAR-T cells have enhanced capability to persist and expand during and after leukemia control. FIG. 19A Cells were recovered from the bone marrow (BM) and liver of leukemia NSG mice treated with ID3^{OE}-CAR T cells or control (Con)-CAR T cells at day 14 after transplantation. FIG. 19B and FIG. 19C show flow cytometric analysis of TIM3 and PD-1 treated with control and Id3OE in bone marrow (FIG. 19B) and liver (FIG. 19C). FIG. 19D, FIG. 19E, and FIG. 19F show results of ex vivo reculture of control and Id3^{OE} T cells (3-4 days). Cells were cultured ex vivo in the presence of cytokines for 3 days and analyzed using flow cytometry (FIG. 19D and FIG. 19E), or restimulated with tumor cells to measure their proliferative capacity FIG. 19F is a graph showing tumor restimulation plotting fold change vs days post stimulation. *, P<0.05; **, P<0.01; ***, P<0.001.

FIG. 20 comprising FIG. 20A, FIG. 20B and FIG. 20C shows that human ID3^{OE}-CAR T cells had augmented memory protection against tumor challenges in human xenograft leukemia NSG mice. FIG. 20A is a schematic of the tumor rechallenge model used. Raji cells were s.c. injected to induce lymphoma. Tumor growth (FIG. 20B) and survival of tumor mice (FIG. 20C) were monitored over time ID3^{OE}-CAR T cell-treated leukemia mice prevented the growth lymphoma under the skin with 7/9 of them surviving without tumor by 55 days after tumor challenge. In contrast, all control mice died from lymphoma within 35 days after tumor challenge. **, P<0.01.

### DETAILED DESCRIPTION

### Glossary

The term “4-1BB” (or “CD137” or tumor necrosis factor receptor super family 9) as used herein refers to an inducible costimulatory receptor expressed on activated T and natural killer (NK) cells. [Chester, C. et al. Blood (2018) 131 (1): 49-57]. There is wide expression of 4-1BB throughout the hematopoietic and nonhematopoietic compartments. 4-1BB is expressed on DC activated monocytes and NK cells, neutrophils, eosinophils and mast cells. [Id., citing Wilcox, R A et al. J. Immunol. (2002) 168 (9): 4262-67; Heinisch IV, et al. Eur. J. Immunol. (2000) 39 (120): 3441-6; Schwarz, H. et al. Blood (1995) 85 (4): 1043-52].

On T cells, 4-1BB is transiently expressed after T-cell receptor engagement and, when 4-1BB is engaged by its natural or artificial ligand, provides CD28-independent costimulation resulting in enhanced proliferation and TH1 cytokine production. [Id., citing DeBenedette, MA., et al. J. Immunol. (1997) 158 (2): 551-9; Saoullli, K., et al. J. Exp. Med. (1998) 187 (11): 1849-62]. The major biological ligand of 4-1BB, 4-1BBL, is expressed on activated APCs, including dendritic cells (DCs) and macrophages and B cells [Id., citing Goodwin, R G., et al. Eur. J. Immunol. (1993) 23 (10): 2631-41; Alderson, M R., et al. Eur. J. Immunol. (1994) 24 (9): 2219-27; Futagawa, T., et al. Intl Immunol. (2002) 14 (3): 275-86; Pollok, K E., et al. Eur. J. Immunol. (1994) 24 (2): 367074]. Ligation of 4-1BB recruits TNFR-associated factor (TRAF) 1 and TRAF2 and induces signaling through the master transcription factor NF-xB and MAPKs. [Id., citing Lee, D Y., et al. PLoS One (2013) 8 (7): e69677; Vinay, D S and Kwon, BS. Semin. Immunol. (1998) 10 (6): 481-9]. Upon ligation with agonist mAbs, 4-1BB rapidly internalizes to an endosomal compartment from which it keeps signaling through this pathway. [Id., citing Bradley, J R and Pober, J S. Oncogene (2001) 20 (44): 6482-91; Kim, C M et al. Sci Rep. (2016) 6 (1): 25526]. 4-1BB signaling ultimately contributes to the secretion of IL-2 and IFN-gamma and upregulation of the antiapoptotic Bcl-2 family members Bcl-xL and Bfl-1, which provide strong protection against activation-induced T cell death. [Id., citing Hurtado, J C., et al. J. Immunol. (1997) 158 (6): 2600-9; Lee, H-W., et al. J. Immunol. (2002) 169 (9): 4882-8; Maus, M V., et al. Nat. Biotechnol. (2002) 20 (2): 143-8; Takahashi, C., et al. J. Immunol. (1999) 162 (9): 5037-40]. Even though 4-1BB and CD28 costimulation are said to be functionally independent, CD28 costimulation is a powerful stimulus for 4-1BB upregulation,

The term “adaptive immune response” as used herein refers to an immune response mediated by uniquely specific recognition of a non-self entity by lymphocytes whose activation leads to elimination of the entity and the production of specific memory lymphocytes. Because these memory lymphocytes forestall disease in subsequent attacks by the same entity, the host immune system has “adapted” to cope with the entity.

The term “administering” and its other grammatical forms as used herein refers to the giving or applying of a substance and includes in vivo administration, as well as administration directly to tissue ex vivo. Generally, compositions may be administered systemically either orally, buccally, parenterally, topically, by inhalation or insufflation (i.e., through the mouth or through the nose), or rectally in dosage unit formulations containing the conventional nontoxic pharmaceutically acceptable carriers, adjuvants, and vehicles as desired, or may be locally administered by means such as, but not limited to, injection, implantation, grafting, topical application, or parenterally. The term “parenteral” as used herein refers to introduction into the body by way of an injection (i.e., administration by injection), including, for example, subcutaneously (i.e., an injection beneath the skin), intramuscularly (i.e., an injection into a muscle), intravenously (i.e., an injection into a vein), intrathecally (i.e., an injection into the space around the spinal cord or under the arachnoid membrane of the brain), intrasternal injection or infusion techniques.

The term “allele” as used herein refers to any of one or more alternative forms of a given gene.

The term “alloantigen” as used herein refers to an antigen from a genetically different individual of the same species.

The term “allogeneic” as used herein refers to being derived from a genetically different individual of the same species.

The term “alloreactive” as used herein refers to a strong primary T cell response against allelic variants of major histocompatibility complex (MHC) molecules in a species.

As used herein, the term “antibody” includes, by way of example, both naturally occurring and non-naturally occurring antibodies. Specifically, the term “antibody” includes polyclonal antibodies and monoclonal antibodies, and fragments thereof. Furthermore, the term “antibody” includes chimeric antibodies and wholly synthetic antibodies, and fragments thereof.

As used herein, the term “antibody” is used in the broadest sense and encompasses various antibody structures, including but not limited to monoclonal antibodies, polyclonal antibodies, antibody fragments, chimeric antibodies and wholly synthetic antibodies as long as they exhibit the desired antigen-binding activity. In nature, antibodies are serum proteins the molecules of which possess small areas of their surface that are complementary to small chemical groupings on their targets. These complementary regions (referred to as the antibody combining sites or antigen binding sites) of which there are at least two per whole antibody molecule, and in some types of antibody molecules ten, eight, or in some species as many as 12, may react with their corresponding complementary region on an antigen (the antigenic determinant or epitope) to link several molecules of multivalent antigen together to form a lattice. The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface.

The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface.

Human antibodies show two kinds of light chains, κ and λ; individual molecules of immunoglobulin generally are only one or the other. In mammals, there are five classes of antibodies, IgA, IgD, IgE, IgG, and IgM, each with its own class of heavy chain. All five immunoglobulin classes differ from other serum proteins in that they show a broad range of electrophoretic mobility and are not homogeneous. This heterogeneity—that individual IgG molecules, for example, differ from one another in net charge—is an intrinsic property of the immunoglobulins.

The principle of complementarity, which often is compared to the fitting of a key in a lock, involves relatively weak binding forces (hydrophobic and hydrogen bonds, van der Waals forces, and ionic interactions), which are able to act effectively only when the two reacting molecules can approach very closely to each other and indeed so closely that the projecting constituent atoms or groups of atoms of one molecule can fit into complementary depressions or recesses in the other. Antigen-antibody interactions show a high degree of specificity, which is manifest at many levels. Brought down to the molecular level, “specificity” means that the combining sites of antibodies to an antigen have a complementarity not at all similar to the antigenic determinants of an unrelated antigen. Whenever antigenic determinants of two different antigens have some structural similarity, some degree of fitting of one determinant into the combining site of some antibodies to the other may occur; this phenomenon gives rise to cross-reactions. Cross reactions are of major importance in understanding the complementarity or specificity of antigen-antibody reactions. Immunological specificity or complementarity makes possible the detection of small amounts of impurities/contaminations among antigens.

Monoclonal antibodies (mAbs) can be generated by fusing mouse spleen cells from an immunized donor with a mouse myeloma cell line to yield established mouse hybridoma clones that grow in selective media. A hybridoma cell is an immortalized hybrid cell resulting from the in vitro fusion of an antibody-secreting B cell with a myeloma cell. In vitro immunization, which refers to primary activation of antigen-specific B cells in culture, is another well-established means of producing mouse monoclonal antibodies.

Diverse libraries of immunoglobulin heavy (V_{H}) and light (V_{L}=V_{κ} and V_{λ}) chain variable genes from peripheral blood lymphocytes also can be amplified by polymerase chain reaction (PCR) amplification. Genes encoding single polypeptide chains in which the heavy and light chain variable domains are linked by a polypeptide spacer (single chain Fv or scFv) can be made by randomly combining heavy and light chain V-genes using PCR. A combinatorial library then can be cloned for display on the surface of filamentous bacteriophage by fusion to a minor coat protein at the tip of the phage.

The technique of guided selection is based on human immunoglobulin V gene shuffling with rodent immunoglobulin V genes. The method entails (i) shuffling a repertoire of human V_{L} chains with the heavy chain variable region (V_{H}) domain of a mouse monoclonal antibody reactive with an antigen of interest; (ii) selecting half-human Fabs on that antigen (iii) using the selected V_{L} genes as “docking domains” for a library of human heavy chains in a second shuffle to isolate clone Fab fragments having human light chain genes; (v) transfecting mouse myeloma cells by electroporation with mammalian cell expression vectors containing the genes; and (vi) expressing the V genes of the Fab reactive with the antigen as a complete IgG1 antibody molecule in the mouse myeloma.

The term antibody may include an oligoclonal antibody, a polyclonal antibody, a monoclonal antibody, a chimeric antibody, a CDR-grafted antibody, a multi-specific antibody, a bi-specific antibody, a catalytic antibody, a chimeric antibody, a humanized antibody, a fully human antibody, an anti-idiotypic antibody, and an antibody that can be labeled in soluble or bound form, as well as fragments, variants or derivatives thereof, either alone or in combination with other amino acid sequences provided by known techniques.

An antibody may be from any species. The term antibody also includes binding fragments of the antibodies of the invention. Binding fragments of an antibody can be produced by recombinant DNA techniques, or by enzymatic or chemical cleavage of intact antibodies. Exemplary fragments include Fv, Fab, Fab′, single stranded antibody (svFC), dimeric variable region (Diabody) and di-sulfide stabilized variable region (dsFv). Structural and functional domains can be identified by comparison of the nucleotide and/or amino acid sequence data to public or proprietary sequence databases. For example, computerized comparison methods can be used to identify sequence motifs or predicted protein conformation domains that occur in other proteins of known structure and/or function. Methods to identify protein sequences that fold into a known three-dimensional structure are known. See, for example, Bowie et al. Science 253:164 (1991), which is incorporated by reference in its entirety. An antibody other than a “bispecific” or “bifunctional” antibody is understood to have each of its binding sites identical.

The term “antibody construct” as used herein refers to a polypeptide comprising one or more the antigen-binding portions linked to a linker polypeptide or an immunoglobulin constant domain. Linker polypeptides comprise two or more amino acid residues joined by peptide bonds and are used to link one or more antigen-binding portions. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). An immunoglobulin constant domain refers to a heavy or light chain constant domain. Human IgG heavy chain and light chain constant domain amino acid sequences are known in the art. Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques.

The term “antigen” as used herein refers to a molecule containing one or more epitopes (either linear, conformational or both) that will stimulate a host's immune-system to make a humoral and/or cellular antigen-specific response. The term is used interchangeably with the terms “immunogen” or “epitope.” Normally, a B-cell epitope will include at least about 5 amino acids but can be as small as 3-4 amino acids. A T-cell epitope, such as a CTL epitope, will include at least about 7-9 amino acids, and a helper T-cell epitope will include at least about 12-20 amino acids. Normally, an epitope will include between about 7 and 15 amino acids, such as, 7, 8, 9, 10, 11, 12, 13, 14, or 15 amino acids. The term includes polypeptides which include modifications, such as deletions, additions and substitutions (generally conservative in nature) as compared to a native sequence, as long as the protein maintains the ability to elicit an immunological response, as defined herein. These modifications may be deliberate, as through site-directed mutagenesis, or may be accidental, such as through mutations of hosts which produce the antigens.

The term “antigen binding site” as used herein refers to the structure formed at the amino-terminal ends (variable domains) of the light and heavy chains, which are folded to form 3-dimensional (globular) variable domains, V_{H} and V_{L}. The antigen binding site makes physical contact with the antigen and binds it noncovalently. The antigen specificity of the site is determined by its shape and the amino acids present.

The term “antigen presentation” as used herein, generally refers to the display to a T cell of antigen on the surface of a cell, e.g., in the form of peptide fragments bound to MHC molecules.

As used herein, the term “antigen presenting cell (APC)” refers to a class of cells capable of displaying on its surface (“presenting”) one or more antigens in the form of peptide-MHC complex recognizable by specific effector cells of the immune system, and thereby inducing an effective cellular immune response against the antigen or antigens being presented. Examples of professional APCs are dendritic cells and macrophages, though any cell expressing MHC Class I or II molecules can potentially present peptide antigen. An APC can be an “artificial APC,” meaning a cell that is engineered to present one or more antigens. Before a T cell can recognize a foreign protein, the protein has to be processed inside an antigen presenting cell or target cell so that it can be displayed as peptide-MHC complexes on the cell surface.

As used herein the term “antigen processing” refers to the intracellular degradation of foreign proteins into peptides that can bind to MHC molecules for presentation to T cells.

The term “AP-1” as used herein refers to a heterodimeric transcription factor formed as one of the outcomes of intracellular signaling via the antigen receptors of lymphocytes and the TLRs of cells of innate immunity. Most often AP-1 contains one Fos-family member and one Jun-family member. AP-1 mainly activates the expression of genes for cytokines and chemokines.

The terms “apoptosis” or “programmed cell death” refer to a highly regulated and active process that contributes to biologic homeostasis comprising a series of biochemical events that lead to a variety of morphological changes, including blebbing, changes to the cell membrane, such as loss of membrane asymmetry and attachment, cell shrinkage, nuclear fragmentation, chromatin condensation, and chromosomal DNA fragmentation, without damaging the organism.

Apoptotic cell death is induced by many different factors and involves numerous signaling pathways, some dependent on caspase proteases (a class of cysteine proteases) and others that are caspase independent. It can be triggered by many different cellular stimuli, including cell surface receptors, mitochondrial response to stress, and cytotoxic T cells, resulting in activation of apoptotic signaling pathways.

The caspases involved in apoptosis convey the apoptotic signal in a proteolytic cascade, with caspases cleaving and activating other caspases that then degrade other cellular targets that lead to cell death. The caspases at the upper end of the cascade include caspase-8 and caspase-9. Caspase-8 is the initial caspase involved in response to death domain (DD) containing receptors like Fas.

Receptors in the TNF receptor family are associated with the induction of apoptosis, as well as inflammatory signaling. The Fas receptor (CD95) mediates apoptotic signaling by Fas-ligand expressed on the surface of other cells. The Fas-FasL interaction plays an important role in the immune system and lack of this system leads to autoimmunity, indicating that Fas-mediated apoptosis removes self-reactive lymphocytes. Fas signaling also is involved in immune surveillance to remove transformed cells and virus infected cells. Binding of Fas to oligimerized FasL on another cell activates apoptotic signaling through a cytoplasmic domain termed the death domain (DD) that interacts with signaling adaptors including FAF, FADD and DAX to activate the caspase proteolytic cascade. Caspase-8 and caspase-10 first are activated to then cleave and activate downstream caspases and a variety of cellular substrates that lead to cell death.

Mitochondria participate in apoptotic signaling pathways through the release of mitochondrial proteins into the cytoplasm. Cytochrome c, a key protein in electron transport, is released from mitochondria in response to apoptotic signals, and activates Apaf-1, a protease released from mitochondria. Activated Apaf-1 activates caspase-9 and the rest of the caspase pathway. Smac/DIABLO is released from mitochondria and inhibits inhibitor of apoptosis (IAP) proteins that normally interact with caspase-9 to inhibit apoptosis. Apoptosis regulation by Bcl-2 family proteins occurs as family members form complexes that enter the mitochondrial membrane, regulating the release of cytochrome c and other proteins. TNF family receptors that cause apoptosis directly activate the caspase cascade, but can also activate Bid, a Bcl-2 family member, which activates mitochondria-mediated apoptosis. Bax, another Bcl-2 family member, is activated by this pathway to localize to the mitochondrial membrane and increase its permeability, releasing cytochrome c and other mitochondrial proteins. Bcl-2 and Bcl-xL prevent pore formation, blocking apoptosis. Like cytochrome c, AIF (apoptosis-inducing factor) is a protein found in mitochondria that is released from mitochondria by apoptotic stimuli. While cytochrome c is linked to caspase-dependent apoptotic signaling, AIF release stimulates caspase-independent apoptosis, moving into the nucleus where it binds DNA. DNA binding by AIF stimulates chromatin condensation, and DNA fragmentation, perhaps through recruitment of nucleases.

The mitochondrial stress pathway begins with the release of cytochrome c from mitochondria, which then interacts with Apaf-1, causing self-cleavage and activation of caspase-9. Caspase-3, -6 and -7 are downstream caspases that are activated by the upstream proteases and act themselves to cleave cellular targets.

Granzyme B and perforin proteins released by cytotoxic T cells induce apoptosis in target cells, forming transmembrane pores, and triggering apoptosis, perhaps through cleavage of caspases, although caspase-independent mechanisms of Granzyme B mediated apoptosis have been suggested.

Fragmentation of the nuclear genome by multiple nucleases activated by apoptotic signaling pathways to create a nucleosomal ladder is a cellular response characteristic of apoptosis. One nuclease involved in apoptosis is DNA fragmentation factor (DFF), a caspase-activated DNAse (CAD). DFF/CAD is activated through cleavage of its associated inhibitor ICAD by caspases proteases during apoptosis. DFF/CAD interacts with chromatin components such as topoisomerase II and histone H1 to condense chromatin structure and perhaps recruit CAD to chromatin. Another apoptosis activated protease is endonuclease G (EndoG). EndoG is encoded in the nuclear genome but is localized to mitochondria in normal cells. EndoG may play a role in the replication of the mitochondrial genome, as well as in apoptosis. Apoptotic signaling causes the release of EndoG from mitochondria. The EndoG and DFF/CAD pathways are independent since the EndoG pathway still occurs in cells lacking DFF.

Hypoxia, as well as hypoxia followed by reoxygenation, can trigger cytochrome c release and apoptosis. Glycogen synthase kinase (GSK-3) a serine-threonine kinase ubiquitously expressed in most cell types, appears to mediate or potentiate apoptosis due to many stimuli that activate the mitochondrial cell death pathway. [Loberg, R D, et al., J. Biol. Chem. (2002)277 (44): 41667-673]. It has been demonstrated to induce caspase 3 activation and to activate the proapoptotic tumor suppressor gene p53. It also has been suggested that GSK-3 promotes activation and translocation of the proapoptotic Bcl-2 family member, Bax, which, upon aggregation and mitochondrial localization, induces cytochrome c release. Akt is a critical regulator of GSK-3, and phosphorylation and inactivation of GSK-3 may mediate some of the antiapoptotic effects of Akt.

The term “assay for transposase-accessible chromatin with sequencing” or “ATAC-Seq” as used herein refers to a popular method for determining chromatin accessibility across the genome. By sequencing regions of open chromatin, ATAC-Seq can help uncover how chromatin packaging and other factors affect gene expression.

The term “basic leucine zipper transcription factor ATF-like” or “BATF” as used herein refers to a bZIP transcription factor that plays an important role in regulating differentiation and function in many lymphocyte lineages. [Kurachi, M., et al. Nat. Immunol. (2014) 15 (4): 373-83, citing Schraml, B U., et al. Nat. Immunol. (2006) 7 (12): 1317-25; Betz, B C., et al. J. Exp. Med. (2010) 207 (5): 933-42; Ise, W., et al. Nat. Immunol. (2011) 12 (6): 536-43; Murphy, T L., et al. Nat. Rev. Immunol. (2013) 13 (7): 499-509; Grigoryan, G., et al. Nature (2009) 458 (7240): 859-64]. In the CD8+ T cell lineage, increased expression of BATF in exhausted CD8+ T cells suppresses their effector function [Id., citing Quigley, M., et al. Nat. Med. (2010) 16 (10): 1147-51]. In the CD4⁺ T cell lineage, BATF is required for the differentiation of interleukin 17 (IL-17)-producing helper T cells (TH17)14, where it binds co-operatively with the transcription factor IRF4 [Id., citing Glasmacher, E., et al. Science (2012) 338 (6109): 975-980; Li, P., et al. Nature (2012) 490 (7421): 543-6; Ciofani, M., et al. Cell (2012) 151 (2): 289-303] and its dimerization partners c-Jun, JunB and JunD [Id., citing Grigoryan, G. et al. Nature (2009) 458 (7240): 859-64]. BATF is also important for the development of follicular helper T cells (TFH) by regulating the transcription factors Bcl-6 and c-Maf [Id., citing Betz, B C., et al. J. Exp. Med. (2010) 207 (5): 933-42; Ise, W., et al. Nat. Immunol. (2011) 12 (6): 536-43]. In addition, BATF is required for class-switch recombination in B cells and to regulate activation-induced cytidine deaminase [Id., citing Ise, W., et al. Nat. Immunol. (2011) 12 (6): 536-43] as well as DNA damage checkpoint in hematopoietic stem cell (HSC) self-renewal [Id., citing Wang, J. et al., Cell (2012) 148 (5): 1001-14]. BATF also has a fundamental role in regulating effector CD8+ T cell differentiation. BATF-deficient CD8+ T cells show profound defects in effector expansion and undergo proliferative and metabolic catastrophe early after antigen encounter. BATF, together with IRF4 and Jun proteins, binds to and promotes early expression of genes encoding lineage-specific transcription-factors (T-bet and Blimp-1) and cytokine receptors, while paradoxically repressing genes encoding effector molecules (IFN-γ and granzyme B).

Thus, BATF amplifies TCR-dependent transcription factor expression and augments inflammatory signal propagation but restrains effector gene expression. This checkpoint prevents irreversible commitment to an effector fate until a critical threshold of downstream transcriptional activity has been achieved. [Id.]

The term “beta2-microglobulin” as used herein refers to the light (0) chain of the MHC class I proteins, encoded outside the MHC. It binds noncovalently to the heavy or a chain and is required for MHC class I expression.

The term “binding” and its various grammatical forms means a lasting attraction between chemical substances.

The term “binding specificity” as used herein involves both binding to a specific partner and not binding to other molecules. Functionally important binding may occur at a range of affinities from low to high, and design elements may suppress undesired cross-interactions. Post-translational modifications also can alter the chemistry and structure of interactions. “Promiscuous binding” may involve degrees of structural plasticity, which may result in different subsets of residues being important for binding to different partners. “Relative binding specificity” is a characteristic whereby in a biochemical system a molecule interacts with its targets or partners differentially, thereby impacting them distinctively depending on the identity of individual targets or partners.

The term “CD25” as used herein refers to the α chain of the IL-2 receptor. It is expressed on activated T cells and Treg cells.

The term “CD44” as used herein refers to a cell surface glycoprotein molecule expressed by a variety of lymphoid and nonlymphoid cells. CD44 is a marker of T cell activation and a property of long-lived memory T cells. CD44 molecule participates in cell adhesion and migration, lymphocyte homing, activation and proliferation, lytic activity of T cells and NK cells, and tumor metastasis. Th1 and Th2 lymphocytes express CD44. CD44 deficiency triggers a Th2-biased Th development. [Guan, H. et al. J. Immunol. (2009) 183 (1): 172-80].

The term “cell proliferation” as used herein refers to the process that results in an increase of the number of cells, and is defined by the balance between cell divisions and cell loss through cell death or differentiation.

The term “cell sorting” as used herein refers to a process of cell identification and cell selection and subsequent separation of the different cell species. Cells can be sorted by different characteristics such as morphology but also based on markers. For many cell sorting methods, fluorescently labeled antibodies, which only bind to specific cell types or cells in certain stages of cellular development, are applied to identify the cells of interest and thus to distinguish target cells from unwanted cells. Cell sorting is widely used to obtain a homogeneous cell population from mixed cell samples. One of the most commonly used methods in cell sorting is FACS (fluorescence activated cell sorting). This method relies on cell suspensions which contain target cells that have been specifically labeled with a fluorescent dye via antibodies or that express fluorescent proteins that can be detected by a FACS cell sorter. A similar method to FACS is Magnetic Activated Cell Sorting (MACS), where antibody-coated magnetic beads specifically bind to the target cells to be able to separate them with a strong magnet from non-labeled cells.

The terms “central lymphoid organs”, “central lymphoid tissues,” and “primary lymphoid organs” as used herein refers to the sites of lymphocyte development. In humans, these are the bone marrow and the thymus. B lymphocytes develop in the bone marrow. T lymphocytes develop within the thymus from bone marrow-derived progenitors.

The term “chemokine” as used herein refers to a class of chemotactic cytokines that orchestrate migration and positioning of immune cells within the tissues. Chemokines bind to seven transmembrane G protein-coupled receptors that trigger intracellular signaling that drives cell polarization, adhesion, and migration [Vilgelm, A E and Richmond, A. Front. Immunol. (2019) doi.org/10.3389/fimmu.2019.00333, citing Griffith, J W., et al. Annu. Rev. Immunol. (2014) 32: 659-702; Nagarsheth, N., et al. Nat. Rev. Immunol. (2017) 17: 559-72]. They are divided into four families based upon structure: CXC, CC, CX3C, and C chemokines. The receptors follow a similar nomenclature system, based upon the family of chemokines to which they bind. In addition there is a family of atypical chemokine receptors that do not directly couple to G proteins, but are reported to have a variety of roles in development, homeostasis, inflammatory disease, infection, and cancer [Id., citing Nibbs, RJ, Graham, GJ. Nat. Rev. Immunol. (2013) 13: 815-29]. Chemokines play an essential role in guiding the migration of both activating and suppressive immune cell types. The continuous migration of immune cells between lymphoid and nonlymphoid organs is a key feature of the immune system, facilitating the distribution of effector cells within nearly all compartments of the body. Reaching their correct position within primary, secondary, or tertiary lymphoid organs is a prerequisite to ensure immune cells' unimpaired differentiation, maturation, and selection, as well as their activation or functional silencing. The superfamilies of chemokines and chemokine receptors are of major importance in guiding immune cells to and within lymphoid and nonlymphoid tissues. [Schulz, O., et al. Annu. Rev. Immunol. (2016) 34: 203-42]. Most chemokine receptors are transmembrane-spanning heterotrimeric G-protein-coupled receptors [Kohli, K., et al. Cancer Gene Therapy (2022) 29: 10-21].

The term “chimeric antigen receptor” or “CAR” as used herein refers to a synthetic MHC-independent receptor that targets T cells to a chosen antigen and reprograms T cell function, metabolism, and persistence [Riviere, I. and Sadelain, M. Mol. Ther. (2017) 25 (5): 1117-24, citing Eshhar, Z., et al. Springer Semin. Immunopathol. (1996) 18: 199-2009; Sadalain, M., et al. Nat. Rev. Cancer (2003) 3: 35-45]. A CAR is mainly composed of three parts: an extracellular antigen recognition domain, usually a single-chain variable fragment (scFv) derived from a monoclonal antibody; a spacer/hinge region and transmembrane domain; and an intracellular signal transduction domain. In a first generation CAR, the intracellular signal transduction domain includes a CD3L chain. In a second generation CAR, the intracellular signal transduction domain includes a CD3 (chain and one costimulatory molecule. In a third generation CAR, the intracellular signal transduction domain includes a CD3L chain and two costimulatory molecules. The intracellular signal transduction domain of a fourth generation CARs includes a CD3 (chain and one costimulatory molecule and expresses a cytokine, such as IL-12.

An optimal CAR will specifically bind an antigen expressed exclusively in tumor cells to form an effective immunological synapse leading to downstream T-cell signaling and a potent and specific anti-tumor effect.

Through their extracellular domain, CARs bind cell surface molecules independently of the major histocompatibility complex (MHC), in contrast to the physiological T cell receptor, which engages MHC/peptide complexes. CARs may thus target proteins, carbohydrates, or glycolipids and function independently of patient HLA haplotype. Binding to antigen triggers T cell activation, which is commonly mediated by the cytoplasmic domain of the CD3-ζ chain. [Riviere, I. and Sadelain, M. Mol. Ther. (2017) 25 (5): 1117-24, citing Irving, B A and Weiss, A. Cell (1991) 64: 891-901; Romeo, C., Seed, B. Cell (1991) 64: 1037-46; Letourneur, F. and Klausner, RD. Proc. Natl. Acad. Sci. USA (1991) 88: 8905-9; Eshhar, Z., et al. Proc. Natl Acad. Sci. USA (1993) 90: 720-24; Brocker, T., et al. Eur. J. Immunol. (1993) 23: 1435-9].

The term “clonal expansion” as used herein refers to the proliferation of antigen-specific lymphocytes in response to antigenic stimulation that precedes their differentiation into effector cells. It is an essential step in adaptive immunity, allowing rar antigen-specific cells to increase in number so that they can effectively combat the pathogen that elicited the response.

The term “clonal revival” as used herein refers to reactivation of peripheral T cells sharing the same clonotypes with pre-existing T_{exhausted} clones that can enter a tumor following immune checkpoint blockade [Liu, B. et al. Nature Cancer (2022) 3: 108-121].

The term “clonotype” with respect to T cells refers to a unique nucleotide sequence that arises during the gene rearrangement process for that receptor. The combination of nucleotide sequences for the surface expressed receptor pair would define the T cell clonotype. Clonotyping, the process used to identify the unique nucleotide CDR sequences of a TCR chain, involves PCR amplification of the cDNA using V region-specific primers and either constant region (C) or J region-specific primer pairs, followed by nucleotide sequencing of the amplicon. The diversity index therefore is an expression of the CDR3 clonotype of the T cell R chain out of all the repertoires. It is noted that referring to only one T-cell receptor chain ignores that the actual clonotype of a T-cell consists of the combination of both alpha and beta receptor chains.

The term “clonotypic” as used herein refers to a feature unique to members of a clone. For example, the distribution of antigen receptors in the lymphocyte population is said to be clonotypic, as the cells of a given clone all have identical antigen receptors.

The term “cognate” signifies two biomolecules that typically interact, e.g., a receptor and its cognate ligand.

The term “composition” or “formulation” as used herein refers to a cell product of the described invention that comprises all active and inert ingredients.

The term “costimulation” as used herein refers to the second signal required for completion of lymphocyte activation and prevention of anergy, which is supplied by engagement of CD28 by CD80 and CD86 (T cells) and of CD40 by CD40 Ligand (B cells).

The term “costimulatory molecule” as used herein refers to molecules that are displayed on the cell surface that have a role in enhancing the activation of a T cell that is already being stimulated through its TCR. For example, HLA proteins, which present foreign antigen to the T cell receptor, require costimulatory proteins which bind to complementary receptors on the T cell's surface to result in enhanced activation of the T cell. Co-stimulatory molecules are highly active immunomodulatory proteins that play a critical role in the development and maintenance of an adaptive immune response (Kaufman and Wolchok eds., General Principles of Tumor Immunotherapy, Chpt 5, 67-121 (2007)). The two signal hypothesis of T cell response involves the interaction between an antigen bound to an HLA molecule and with its cognate T cell receptor (TCR), and an interaction of a co-stimulatory molecule and its ligand. Specialized APCs, which are carriers of a co-stimulatory second signal, are able to activate T cell responses following binding of the HLA molecule with TCR. By contrast, somatic tissues do not express the second signal and thereby induce T cell unresponsiveness (Id.). Many of the co-stimulatory molecules involved in the two-signal model can be blocked by co-inhibitory molecules that are expressed by normal tissue (Id.). In fact, many types of interacting immunomodulatory molecules expressed on a wide variety of tissues may exert both stimulatory and inhibitory functions depending on the immunologic context (Id.). The term “co-stimulatory receptor” as used herein refers to a cell surface receptor on naïve lymphocytes through which they receive signals additional to those received through the antigen receptor, and which are necessary for the full activation of the lymphocyte. Examples are CD30 and CD40 on B cells, and CD27 and CD28 on T cells.

The term “CRISPR” (meaning “clustered regularly interspaced short palindromic repeats”) —“Cas” (meaning “CRISPR-associated”) refers to a class of enzymes derived from bacteria used to selectively modify the DNA of living organisms. In brief, CRISPR “spacer” sequences are transcribed into short RNA sequences (“CRISPR RNAs” or “crRNAs”) capable of guiding the system to matching sequences of DNA. When a target DNA is found, Cas9—one of the enzymes produced by the CRISPR system—binds to the DNA and cuts it, shutting the targeted gene off. Since the CRISPR-Cas9 system itself is capable of cutting DNA strands, CRISPRs do not need to be paired with separate cleaving enzymes as other tools do. They can also be matched with tailor-made “guide” RNA (gRNA) sequences designed to lead them to their DNA targets.

The term “CTLA-4” or “cytotoxic T-lymphocyte-associated antigen 4” or “CD152” as used herein refers to an inhibitory receptor and immune checkpoint that aids in maintaining self-antigen immunity by dampening T cell responses. It is a counterereceptor to costimulatory molecule CD28.

The term “culture” and its other grammatical forms as used herein, is meant to refer to a process whereby a population of cells is grown and proliferated on a substrate in an artificial medium.

The term “cytokine” as used herein refers to small soluble protein substances secreted by cells which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines. Non-limiting examples of cytokines include e.g., IL-1, IL-2, IL-3, IL-4, IL-5, IL-6, IL-7, IL-8, IL-9, IL-10, IL-11, IL-12/IL-23 P40, IL13, IL-15, IL-15/IL15-RA, IL-17, IL-18, IL-21, IL-23, TGF-β, IFNγ, GM-CSF, Groa, MCP-1 and TNF-α.

The term “cytotoxic T lymphocytes” (CTLs) as used herein refers to effector CD8+ T cells. Cytotoxic T cells kill by inducing their targets to undergo apoptosis/programmed cell death via extrinsic and intrinsic pathways.

The term “dendritic cells (DC)” as used herein refers to professional antigen presenting cells, which induce naïve T cell activation and effector differentiation. [Patente, T A., et al., Frontiers Immunol. (2019) doi.org/10.3389/fimmu.2018.03176]. Human DCs are identified by their high expression of major histocompatibility complex (MHC) class II molecules (MHC-II) and of CD11c, both of which are found on other cells, like lymphocytes, monocytes and macrophages [Id., citing Carlens J., et al. J Immunol. (2009) 183:5600-7; Drutman S B., et al. J Immunol. (2012) 188:3603-10; Hochweller K S., et al. Eur J Immunol. (2008) 38:2776-83; Huleatt J W., Lefrangois L. J Immunol. (1995) 154:5684-93; Rubtsov A V., et al. Blood (2011) 118:1305-15; Probst H C., et al. Clin Exp Immunol. (2005) 141:398-404; Vermaelen K, Pauwels R. Cytometry (2004) 61A:170-7]. DCs express many other molecules which allow their classification into various subtypes. Although some of the DC subtypes were originally described as macrophages, DC and macrophages have distinct characteristics [Id., citing Delamarre L, Science (2005) 307:1630-4; Geissmann. F, et al. Science (2010) 327:656-61; van Montfoort N., et al. Proc Natl Acad Sci USA. (2009) 106:6730-5] and ontogeny, so that, currently, little doubt remains that they belong to distinct lineages [Id., citing Haniffa M., et al. (2013) 120:1-49; Hashimoto D., et al. Immunity (2013) 38:792-804; Hettinger J., et al. Nat Immunol. (2013) 14:821-30; McGovern N., et al. Immunity (2014) 41:465-77; Naik S H., et al. Nature (2013) 496:229-32; Schulz C., et al. Science (2012) 336:86-90; Schraml B U., et al. Cell (2013) 154:843-58; Wang J., et al. Mol Med Rep. (2017) 16:6787-93; Yona S., et al. Immunity (2013) 38:79-91].

The term “derived from” as used herein encompasses any method for receiving, obtaining, or modifying something from a source of origin.

The term “detectable marker” encompasses both selectable markers and assay markers. The term “selectable markers” refers to a variety of gene products to which cells transformed with an expression construct can be selected or screened, including drug-resistance markers, antigenic markers useful in fluorescence-activated cell sorting, adherence markers such as receptors for adherence ligands allowing selective adherence, and the like. “Assay markers” are measurable components whose presence or absence can be detected and correlated to a particular detectable response.

The term “detectable response” as used herein, is meant to refer to any signal or response that may be detected in an assay, which may be performed with or without a detection reagent. Detectable responses include, but are not limited to, radioactive decay and energy (e.g., fluorescent, ultraviolet, infrared, visible) emission, absorption, polarization, fluorescence, phosphorescence, transmission, reflection or resonance transfer. Detectable responses also include chromatographic mobility, turbidity, electrophoretic mobility, mass spectrum, ultraviolet spectrum, infrared spectrum, nuclear magnetic resonance spectrum and x-ray diffraction. Alternatively, a detectable response may be the result of an assay to measure one or more properties of a biologic material, such as melting point, density, conductivity, surface acoustic waves, catalytic activity or elemental composition. A “detection reagent” is any molecule that generates a detectable response indicative of the presence or absence of a substance of interest. Detection reagents include any of a variety of molecules, such as antibodies, nucleic acid sequences and enzymes. To facilitate detection, a detection reagent may comprise a marker.

The term “derived from” as used herein, is meant to encompasses any method for receiving, obtaining, or modifying something from a source of origin.

The term “differential gene expression” as used herein refers to an observed difference or change in expression level of a gene between two experimental conditions that is statistically significant.

The term “differentiate” and its various grammatical forms as used herein refer to the process of development with an increase in the level of organization or complexity of a cell or tissue, accompanied with a more specialized function.

The term “domain” as used herein refers to a region of a protein with a characteristic tertiary structure and function and to any of the three-dimensional subunits of a protein that together make up its tertiary structure formed by folding its linear peptide chain.

The term “ectopic gene expression” refers to expression of a gene in a place or at a time in which it is not expressed in nature.

The term “effector cell” as used herein refers to a cell that carries out a final response or function. The main effector cells of the immune system, for example, are activated lymphocytes and phagocytes.

The term “effector functions” as used herein refers to the actions taken by effector cells and antibodies to eliminate foreign entities, and includes, without limitation, cytokine secretion, cytotoxicity, and antibody-mediated clearance.

The term “eligible subject” as used herein refers to a subject that satisfies the requirements to be treated with the immunotherapy of the present disclosure under the professional judgment of the patient's physician. Eligibility criteria may include the subject's age, type and stage of cancer, current health status, medical history, and previous treatments.

The term “enrich” as used herein refers to increasing the proportion of a desired substance, for example, to increase the relative frequency of a subtype of cell compared to its natural frequency in a cell population. Positive selection, negative selection, or both are generally considered necessary to any enrichment scheme. Exemplary selection methods include, without limitation, magnetic separation and FACS.

The term “epigenetic marks” as used herein refer to DNA methylation, histone modifications, chromatin remodeling and microRNA.

The term “epigenetics” as used herein refers to a stably heritable phenotype resulting from changes in a chromosome without alterations in the DNA sequence.

The terms “excipient”, “carrier”, or “vehicle” are used interchangeably to refer to carrier materials suitable for formulation and administration of the pharmaceutically acceptable compositions described herein. Carriers and vehicles useful herein include any such materials know in the art which are nontoxic, do not abrogate the biological activity and properties of the cell product of the composition, and do not interact with other components.

The excipient can be inert, or it can possess pharmaceutical benefits.

The term “expand” or “amplify” as used herein with respect to cells refers to increasing in cell number.

As used herein, the term “expression” and its other grammatical forms refers to production of an observable phenotype by a gene, usually by directing the synthesis of a protein. It includes the biosynthesis of mRNA, polypeptide biosynthesis, polypeptide activation, e.g., by post-translational modification, or an activation of expression by changing the subcellular location or by recruitment to chromatin.

The term “flow cytometry” as used herein, refers to a tool for interrogating the phenotype and characteristics of cells. It senses cells or particles as they move in a liquid stream through a laser (light amplification by stimulated emission of radiation)/light beam past a sensing area. The relative light-scattering and color-discriminated fluorescence of the microscopic particles is measured. Flow analysis and differentiation of the cells is based on size, granularity, and whether the cell is carrying fluorescent molecules in the form of either antibodies or dyes. As the cell passes through the laser beam, light is scattered in all directions, and the light scattered in the forward direction at low angles (0.5°-10°, inclusive) from the axis is proportional to the square of the radius of a sphere and so to the size of the cell or particle. Light may enter the cell; thus, the 90° light (right-angled, side) scatter may be labeled with fluorochrome-linked antibodies or stained with fluorescent membrane, cytoplasmic, or nuclear dyes. Thus, the differentiation of cell types, the presence of membrane receptors and antigens, membrane potential, pH, enzyme activity, and DNA content may be facilitated. Flow cytometers are multiparameter, recording several measurements on each cell; therefore, it is possible to identify a homogeneous subpopulation within a heterogeneous population (Marion G. Macey, Flow cytometry: principles and applications, Humana Press, 2007). Fluorescence-activated cell sorting (FACS), which allows isolation of distinct cell populations too similar in physical characteristics to be separated by size or density, uses fluorescent tags to detect surface proteins that are differentially expressed, allowing fine distinctions to be made among physically homogeneous populations of cells.

The term “GATA binding protein 3” (or “GATA-3”) is a transcription factor protein that contains two zinc fingers. It is a member of the GATA family of conserved zinc-finger transcription factors. GATA-3 is a master regulator of type 2 T helper cell development.

The term “gene” as used herein refers to a region of DNA that includes the entire functional unit encompassing coding DNA sequences, noncoding regulatory DNA sequences, and introns, which controls a discrete hereditary characteristic, usually corresponding to a single protein or RNA.

The term “gene activator protein” as used herein regers to a gene regulatotry protein that when bound to its regulatory sequence in DNA activates transcription.

The term “gene editing” as used herein refers to alteration of the genetic material of a living organism by inserting, replacing, or deleting a DNA sequence, typically with the goal of improving some characteristic of the organism.

The term “gene regulatory protein” as used herein refers to any protein that binds to a specific DNA sequence to alter the expression of a gene.

The term “gene repressor protein” as used herein refers to a gene regulatory protein that prevents the initiation of transcription.

The term “Gene Set Enrichment Analysis” (or “GSEA”) as used herein refers to a computational method that determines whether an a priori defined set of genes shows statistically. significant, concordant differences between two biological states. (e.g. phenotypes).

The term “general transcription factor” as used herein refers to any of the proteins whose assembly around the TATA box is required for the initiation of transcription of most eukaryotic genes.

The term “graft versus host disease” or “GVHD” as used herein refers to an attack on the tissues of a recipient by mature T cells from a nonidentical donor, which can cause a variety of symptoms, sometimes severe.

The term “graft versus tumor effect” as used herein refers to immune reactivity mediated by donor T cells against the recipient's tumor cells.

The term “haplotype” as used herein refers to the set of MHC alleles contained on a single chromosome of an individual.

The term “healthy subject” or “healthy donor” or “healthy adult donor” or “healthy control” as used herein refers to an individual having no signs or symptoms of a cancer.

The term “hematopoietic stem cell transplantation” as used herein refers to the process and intravenous infusion of hematopoietic stem and progenitor cells derived from a donor to a recipient in order to restore normal hematopoiesis and/or treat malignancy.

The term “Id3” as used herein refers to a helix-loop-helix factor that controls T cell development and function through antagonizing the function of the E box binding family of transcription factors, which are master regulators of thymic development and antigen-driven T cell responses. Id3 promotes CD8⁺ T cell-, γδ T cell- and iNKT cell-development, but antagonizes CD4⁺ T cell fate determination. Id3 also regulates antigen-specific CD8⁺ T cell memory formation, memory recall response and effector survival, and is associated with CD4⁺ memory TH1 cells. Additionally, Id3 expression may identify precursor exhausted CD8⁺ T cells during chronic infections and cancer.

IFN-regulatory factor (IRF) family of transcription factors. The IFN-regulatory factor (IRF) family of transcription factors includes nine members in mammals that bind to related target-gene sequences [Bollig, N., et al. Proc. Natl Acad. Sci. USA (2012) 109 (22): 8664-9, citing Lohoff, M. and Mak, TW. Nat. Rev. Immunol. (2005) 5: 125-35]. In the T lineage, IRF1 is decisive for TH1 cell generation because it is ubiquitously expressed and redundantly addresses many genes with independent TH1-supporting function [Id., citing Lohoff, M. and Mak, TW, Nat. Rev. Immunol. (2005) 5: 125-35; Lohoff, M. et al. Immunity (1997) 6: 681-9; Taki, S. et al. Immunity (1997) 6: 673-9]. IRF4 has been characterized as an important transcription factor for differentiation of T_{H}2, T_{H}9 and T_{H}17 cells [Id., citing Brustle, A. et al. Nat. Immunol. (2007) 8: 958-66; Huber, M., et al. Proc. Natl Acad. Sci. USA (2008) 105: 20846-51; Lohoff, M., et al. Proc. Natl Acad. Sci. USA (2002) 99: 11808-12; Rengarajan, J., et al. J. Exp. Med. (2002) 195: 1003-12]. In addition, aspects of Treg cell function entirely depend on IR4 [citing, Zheng, Y., et al. Nature (2009) 458: 351-6; Chen, Q., et al. Immunity (2008) 29: 899-911; Staudt, V., et al. Immunity (2010) 33: 192-202]. Treg-specific IRF4 deficiency or lack of IRF4 binding protein leads to a generalized autoimmune syndrome [Id., citing Zheng, Y., et al. Nature (2009) 458: 351-6; Chen, Q., et al. Immunity (2008) 29: 899-911]. IRF4 also plays a role in the development of T_{F}H cells, which are mainly responsible for the intricate organization of T-B interactions and antibody maturation in vivo. Irf4−/− Mice fail to generate germinal centers and fail to generate TFH dells. In the B-cell lineage, IRF4 is important for plasma cell differentiation and isotype switching [Id., citing Klein, U., et al. Nat. Immunol. (2006) 7: 773-82; Sciammas, R. et al. Immunity (2006) 25: 225-36].

As used herein, the term “immune checkpoints” refers to the array of inhibitory pathways necessary for maintaining self-tolerance and that modulate the duration and extent of immune responses to minimize damage to normal tissue. In T cells, the ultimate amplitude and quality of the immune response, which is initiated through antigen recognition by the TCR, is regulated by a balance between co-stimulatory and inhibitory signals (immune checkpoints). [Pardoll, DM. Nat. Rev. Cancer (2012) 12(4): 252-64]. Immune checkpoint molecules such as PD-1, PD-L1, CTLA-4 are cell surface signaling receptors that play a role in modulating the T-cell response in the tumor microenvironment. Tumor cells have been shown to utilize these checkpoints to their benefit by up-regulating their expression and activity. With the tumor cell's ability to commandeer some immune checkpoint pathways as a mechanism of immune resistance, it has been hypothesized that checkpoint inhibitors that bind to molecules of immune cells to activate or inactivate them may relieve their inhibition of an immune response. Immune checkpoint inhibitors have been reported to block discrete checkpoints in an active host immune response allowing an endogenous anti-cancer immune response to be sustained. Recent discoveries have identified immune checkpoints or targets, like PD-1, PD-L1, PD-L2, CTLA-4, TIGIT, TIM-3, LAG-3, CCR4, OX40, OX40L, IDO, and A2AR, as proteins responsible for immune evasion.

The terms “immune escape” or “immune evasion” as used herein refers to a strategy to evade a host's immune response. It is characterized by the inability of the immune system to eliminate transformed cells prior to and after tumor development. The host's contribution is manifested by its inability to recognize antigens expressed by tumor cells, a phenomenon known as “host ignorance.” It happens because of defects in both the innate and adaptive arms of the immune system. The tumor's contribution is manifested by the adaptation of tumor cells to evade the immune system or by developing a microenvironment that suppresses the immune system. [Qian J. et al. (2011) Immune Escape. In: Schwab M. (eds) Encyclopedia of Cancer. Springer, Berlin, Heidelberg. https://doi.org/10.1007/978-3-642-16483-5_2975].

The terms “immune response” and “immune-mediated” are used interchangeably herein to refer to any functional expression of a subject's immune system, against either foreign or self-antigens, whether the consequences of these reactions are beneficial or harmful to the subject. A “primary immune response” is the adaptive immune response that follows the first exposure to a particular antigen. When B and T cells replicate during the primary immune response, they produce T and B effector cells and long-lived antigen-specific memory cells. A “secondary immune response” or “memory recall response” is the immune response that occurs in response to a subsequent exposure to an antigen generated by the reactiveation of memory lymphocytes. In comparison with the primary response, it starts sooner after exposure, produces greater levels of antibody, and procudes class-switched antibodies. Memory T cells are antigen-experienced cells that mediate a faster and more potent response upon repeat encounter with antigen. The term “immunological response” to an antigen or composition as used herein is meant to refer to the development in a subject of a humoral and/or a cellular immune response to an antigen. For purposes of the present disclosure, a “humoral immune response” refers to an immune response mediated by antibody molecules, while a “cellular immune response” is one mediated by T-lymphocytes and/or other white blood cells. One aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of T cells, suppressor T-cells and/or γδ T-cells directed specifically to an antigen or antigens present in the composition of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

In both normal and pathologic pulmonary responses, the respiratory immune system is tailored to respond to different classes of pathogens and allergens optimally through disparate immune reactions commonly known as type 1, type 2, and type 3 immunity [Ma, Q. Front. Immunol. (2020) 11: 1060, citing Spellberg, B. and Edwards, JE, Jr. Clin. Infect. Dis. (2001) 32: 76-102; Annunziato, F., et al. J. Allergy Clin. Immunol. (2015) 135: 626-35]0.29, 30). In many chronic disease conditions and during non-infectious exposures, inflammation often predominates the immune responses. In these cases, the immune responses are sometimes called type 1, type 2, and type 3 inflammation, respectively.

Type 1 and type 2 responses are mutually suppressive to each other in many cases, which helps orchestrate the temporal development of host responses to infection and inflammatory instigators.

Type 1 immunity is characterized by Th1 cells and ILC group 1 cells, which secrete interferon (IFN) 7, IL-2, and lymphotoxin-α. Type 1 responses protect against intracellular microbes through activated mononuclear phagocytes, i.e., M1 macrophages, and an array of proinflammatory cytokines, eicosanoids, and reactive oxygen species (ROS) and reactive nitrogen species (RNS), to stimulate acute inflammation and bacterial killing. Heightened type 1 responses cause excessive damage to lung tissue and contribute to disease pathogenesis, including releasing self-antigens that induce autoimmune reactions. [Id.]

Type 2 immunity consists of TH2 cells, ILC2s, and M2 macrophages, which secrete type 2 cytokines, such as IL-4, IL-5, IL-9, IL-10, and IL-13. These cytokines recruit and activate type 2 effector cells, including eosinophils, basophils, mast cells, and myofibroblasts. Some T_{H}2 cells migrate to lymph node follicles and promote IgE class switch and B cell activation and, hence, are called follicular helper T cells (TFH s). Type 2 responses protect against helminth infection, venoms, and allergens under physiological conditions but, when dysregulated, lead to atopic responses, such as asthma and anaphylaxis. Recent studies reveal that ILC2s can be activated in response to a wide range of stimuli. Moreover, activated ILC2s secrete copious amounts of type 2 cytokines prior to TH2 activation Id., citing Pulendran, B. and Artis, D. Science (2012) 337: 431-5]. These findings suggest a mechanism by which type 2 responses can be initiated in the absence of apparent antigenic stimulation. Functionally, type 2 immunity has been traditionally associated with allergic responses, host defense against helminth infection, and tissue repair. Recent findings suggest a contemporary view of type 2 functions in which type 2 responses seemingly play a more general role in defense against noxious environmental stimuli besides mediating host immunosurveillance at barrier sites. [Id.] In this context, type 2 reactions help eliminate, restrict, and neutralize noxious environmental substances and triggers, such as allergens, as well as repair tissue damage and minimize inflammation at surface tissue. [Id.] Key to the function of type 2 responses in tissue regeneration, wound healing, and suppression of type 1 inflammation is the production of transforming growth factor (TGF) R by type 2 cells, such as M2 macrophages. [Id.] Additionally, ILC2s, eosinophils, and type 2 cytokines are vital regulators of adipose precursor number and fate and the overall adipose tissue homeostasis [Id., citing Lee, M W., et al. Cell (2015) 160: 74-87]. This innate type 2 immune metabolic circuit regulates energy metabolism and, thereby, controls insulin sensitivity and lean physiology [Id., citing Brestoff, J R., et al. Nature (2015) 519: 242-6]. Therefore, the current view of the biology and scope of type 2 immunity has expanded considerably beyond the traditionally recognized type 2 responses [Lloyd, C M and Snelgrove, RJ. Sci. Immunol. (2018) 3: eaat1604].

Apart from this widely accepted paradigm of type 1 and type 2 immunity, a type 3 immune response has been implicated in pulmonary immunity. Type 3 immunity is characterized by TH17 cells and ILC3s, and the production of IL-17 and IL-22 cytokines. Type 3 immune responses protect against extracellular bacteria and fungi through mononuclear phagocytes and neutrophils at mucous membrane epithelia [Id., citing Annunziato, F., et al. J. Allergy Clin. Immunol. (2015) 135: 626-35].

Central to these three types of immunity/inflammation is the polarization of several major immune cells, including T lymphocytes, macrophages, and ILCs, induced by microbial signals, allergens, sterile insults, and microenvironmental cues from damaged tissue. [Id.]

In addition to type 1, type 2, and type 3 responses, Treg lymphocytes can be enriched to regulate immune responses by controlling the polarization of T_{H}1, T_{H}2, and T_{H}17 effector T (Teff) cells and hence the balance among these immune responses. [Id.]

The term “immunological repertoire” refers to the collection of transmembrane antigen-receptor proteins located on the surface of T and B cells. (Benichou, J., et al. Immunology (2011) 135: 183-191). The combinatorial mechanism that is responsible for encoding the receptors does so by reshuffling the genetic code, with a potential to generate more than 1018 different T cell receptors (TCRs) in humans (Id., citing Venturi, Y., et al. Nat. Rev. Immunol. (2008) 8: 231-8), and a much more diverse B-cell repertoire. These sequences, in turn, will be transcribed and then translated into protein to be presented on the cell surface. The recombination process that rearranges the gene segments for the construction of the receptors is key to the development of the immune response, and the correct formation of the rearranged receptors is critical to their future binding affinity to antigen. (Id.) The highly diverse junctional region of the TCR chain, also known as the complementarity-determining region 3 (CDR3) is an important determinant of antigen recognition. [Aversa, I., et al. (2010) Int. J. Mol. Sci. 21: 238; doi:10.3390/ijms21072378, citing Xu, J L and Davis, MM. Immunity (2000) 13: 37-45]. The CDR3 sequence is essentially unique for each newly formed T cell, since it is highly unlikely that two T cells will express the same CDR3 nucleotide sequence [Id., citing Turner, S J., et al. Nat. Rev. Immunol. (2006) 6: 883-94]. At the same time, when a T cell is activated and undergoes a clonal expansion, all the cells of the clonal lineage are equipped with an identical CDR3, which therefore acts as a natural identifier of the clonality of the lymphocytes [Id., citing Kirsch, I., et al. Mol. Oncol. (2015) 9: 2063-700].

The term “immune system” as used herein refers to the body's system of defenses against disease, which comprises the innate immune system and the adaptive immune system. The innate immune system provides a non-specific first line of defense against pathogens. It comprises physical barriers (e.g. the skin) and both cellular (granulocytes, natural killer cells) and humoral (complement system) defense mechanisms. The reaction of the innate immune system is immediate, but unlike the adaptive immune system, it does not provide permanent immunity against pathogens. The adaptive immune response is the response of the vertebrate immune system to a specific antigen that typically generates immunological memory.

The term “immunological synapse” (“IS”) as used herein refers to a highly structured body that functions to concentrate T cell signaling in a defined area. It is associated with the selective recruitment of signaling molecules and exclusion of negative regulators. The synapse is stabilized by a ring of adhesion molecules, including, for example, LFA1, which binds to ICAM1 on the APC. The immune synapse modulates TCR signaling by several mechanisms. In the earliest stages of immune synapse formation, TCR-containing microclusters are recruited to the central molecular cluster. The TCR responds to two distinct pMHC ligands (agonist and self-peptide-MHC) in co-agonism rather than nonspecific TCR-MHC interactions, which adds to the overall binding strength of the TCR-p-MHC complex. The strength of the TCR-p-MHC interactions has a role in determining the influence of coreceptors that are recruited to the immune synapse (e.g., CD8). The immune synapse also modulates TCR signaling by regulating interactions between kinases in the TCR pathway and their substrates. [Morris, G. and Allen, P M. Nature Immunol. (2012) doi:10.1038/nm.2190].

The T cell signaling pathway includes proximate signaling (including phosphorylation of the invariant signaling protein CD3 and early signaling molecules, calcium-mediated signaling (release of intracellular Ca2⁺ stores and influx of extracellular Ca2⁺), and GTPase Ras-MAPK signaling. [Id.]

T cell activation is mediated through highly organized and dynamic interaction of TCRs with MHC-peptide complexes at the IS. A mature IS is an aggregation of TCR-based signalosomes (meaning multimolecular complexes) that induce T cell responses and is defined by three concentric rings of clustered molecules. The inner circle is termed the central supramolecular activation cluster (cSMAC) where TCR signaling takes place. The cSMAC contains most of the TCR-MHC peptide complexes, CD28, PKC-theta and Lck, whereas peripheral SMAC (pSMAC) contains proteins involved in cell adhesion, such as integrin LFA-1, cytoskeletal linker talin, and ICAM1. Larger molecules, such as CD43 and CD45, are excluded from the pSMAC and make up the distal SMAC (dSMAC). Inhibitory and costimulatory molecules, such as PD-1, CTLA-4, and ICOS also are aggregated at the region of the IS and play crucial roles in the regulation of T cell activation. [Watanabe, K., et al. Front. Immunol. (2018) 9: 2486, citing Yokosuka, T., et al. Immunity (2010) 33: 326-39]. Secretion of lytic granules occurs within a secretary synapse between CTLs and target cells. The secretory synapse has two separate and distinct domains in cSMAC: one is a signaling domain, which contains the signaling proteins, and another is a secretory domain for exocytosis of cytokines, perforins and granzymes. The transient polarization and docking of the centrosome to the plasma membrane, which is controlled by Lek signaling, has an important role in the mechanism of directing this secretion. [Id., citing Stinchcombe, J C., et al. Nature (2006) 443: 462-5; Stinchcombe, J C., et al. Nature (2006) 443: 462-5; Tsun, A., et al. J. Cell Biol. (2011) 192: 663-74]

The terms “immunomodulatory”, “immune modulator”, “immunomodulatory,” and “immune modulatory” are used interchangeably herein to refer to a substance, agent, or cell that is capable of augmenting or diminishing immune responses directly or indirectly, e.g., by expressing chemokines, cytokines and other mediators of immune responses.

The term “immunotherapy” as used herein refers to measures taken using immunological methods and principles to target the hyper or hyo-immune state of an organism, intervene or adjust the organism's immune function artificially, and strengthen or attenuate the immune response so as to treat disease. It enhances the immune system's ability to recognize, target and eliminate cancer cells in the body. [Zhang, Z., et al. Front. Immunol. (2021) 12: Barbari, C., et al. Intl J. Mol. Sci. (2020) 21: 5009]. Some types of immunotherapy only target certain cells of the immune system. Others affect the immune system in a general way.

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents.

The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity.’

The term Ingenuity Pathways Analysis” or “IPA” refers to a web-based bioinformatics application that allows researcher to upload data analysis results from high-throughput experiments for functional analysis, integration and further understanding. Pathway analysis (PA) algorithms, combined with large molecular interaction databases, allow for the processing of high-throughput genomic (or proteomic) data, such as those from gene expression microarray or RNA-seq experiments and the rank ordering of perturbed (up or down regulated pathways) associated with an experimental (or clinical) condition. [Yu, C., et al. BMC Bioinformatics (2017) 18: 453, citing Haw, R., et al. Proteomics (2011) 11 (18): 3598-613; Dutta, B., et al. Source Code Biol. Med. (2012) 7(1): 10]. The strategy for pathway analysis involves the use of two mutually complementary metrics, recall and discrimination. Recall measures the consistency of the perturbed pathways identified by applying a particular analysis method to an original large dataset and those identified by the same method to a sub-dataset of the original dataset. [Yu, C. et al. BMC Bioinformatics (2017) 18: 453]. In contrast, discrimination measures specificity—the degree to which the perturbed pathways identified by a particular method to a dataset from one experiment differ from those identifying by the same method to a dataset from a different experiment; i.e., discrimination considers only those pathways that are specific to particular experimental conditions and does not directly reflect the overall extent to which all significant pathways are sufficiently represented in each condition. [Yu, C., et al. BMC Bioinformatics (2017) 18: 453].

The term “inhibitor receptor lymphocyte activation gene-3” or “LAG-3” as used herein refers to a member of the immunoglobulin superfamily (IgSF), which binds to major histocompatibility complex (MHC) class II. LAG-3 expression on tumor infiltrating lymphocytes (TILs) is associated with tumor-mediated immune suppression.

The term “innate immune response” as used herein refers to the various mechanisms encountered by a pathogen or transformed cell before adaptive immunity is induced, such as anatomical barriers, antimicrobial peptides, the complement system, and macrophages and neutrophils carrying nonspecific pattern-recognition receptors. Innate immunity is present in all individuals at all times, does not increase with repeated exposure, and discriminates between groups of similar pathogens, rather than responding to a particular pathogen.

The term “innate lymphoid cells” or “ILCs” as used herein refers to a class of innate immune cells having overlapping characteristics with T cells but lacking an antigen receptor. They arise in several groups, ILC1, ILC2, ILC3, and NK cells, which exhibit properties roughly similar to T_{H}1, T_{H}2, T_{H}17 and CD8 T cells, respectively.

The term “isoform” refers to a version of a protein that has the same function as another protein but that has some small difference(s) in its sequence.

The term “isolated” is used herein to refer to material, such as, but not limited to, a nucleic acid, peptide, polypeptide, or protein, which is: (1) substantially or essentially free from components that normally accompany or interact with it as found in its naturally occurring environment. The terms “substantially free” or “essentially free” are used herein to refer to considerably or significantly free of, or more than about 90% free, about 91% free, about 92% free, about 93% free, about 94% free; about 95% free of, about 96% free, about 97% free, about 98% free or more than about 99% free. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material may be performed on the material within, or removed, from its natural state. For example, a naturally occurring nucleic acid becomes an isolated nucleic acid if it is altered, or if it is transcribed from DNA that has been altered, by means of human intervention performed within the cell from which it originates. Likewise, a naturally occurring nucleic acid (for example, a promoter) becomes isolated if it is introduced by non-naturally occurring means to a locus of the genome not native to that nucleic acid.

The term “Kaplan-Meier survival curve” or “survival curve” as used herein refers to the probability of surviving in a given length of time while considering time in many small intervals. It is commonly used to analyze time-to-event (survival) data, such as the time until death or the time until a specific event occurs. Time is plotted on the x-axis and the survival rate is plotted on the y-axis. Each subject is characterized by three variables: (1) their serial time; (2) their status at the end of their serial time (occurrence of an event of interest or censored); and (3) the study group they are in. “Serial time” refers to the clinical course duration for each subject having a beginning and an end along the time line of the complete study. An “interval”, which is graphed as a horizontal line, is the serial time duration of known survival. An interval therefore is terminated only by the event of interest. “Censoring” means the total survival time for that subject cannot be accurately determined; this can happen when something negative for the study occurs, such as the subject drops out, is lost to follow-up, or required data is not available, or, conversely, something good happens, such as the study ends before the subject had the event of interest occur. Censoring can occur within the study or terminally at the end. Censored subjects are indicated as tick marks; these do not terminate the interval. [Rich, J T., et al. Otolaryngol. Head Neck Surg. (2010) 143 (3): 331-36]. The Kaplan Meier plot assumes that: (i) at any time subjects who are censored (i.e., lost) have the same survival prospects as subjects who continue to be followed; (ii) the survival probabilities are the same for subjects recruited early and late in the study; and (iii) the event (e.g., death) happens at the time specified. Probabilities of occurrence of an event are computed at a certain point of time with successive probabilities multiplied by any earlier computed probabilities to get a final estimate. The survival probability at any particular time is calculated as the number of subjects surviving divided by the number of subjects at risk. Subjects who have died, dropped out, or have been censored from the study are not counted as at risk.

The term “knockout mouse” or “KO” as used herein refers to a mutant mouse strain in which a single gene in the DNA of a mouse embryo is deliberately deleted or rendered defective by genetic engineering techniques.

The term “leukemia” as used herein refers to the clonal expansion of leukemic cells in the bone marrow, classically resulting in elevated numbers of cells of the affected lineage in circulating blood and, with certain lymphoid malignancies, abnormal cellular proliferation in lymphatic tissue [Bispo, J A B., et al. Cold Spring Harb. Perspect. Med. (2020) 10 (6): a034819]. Leukemias are generally classified into subtypes defined by cell lineage (lymphocytic or myeloid) and stage of maturation arrest (acute or chronic). Lymphocytic leukemias start in lymphoid cells while myelogenous leukemias start in myeloid cells. The types of leukemia include: (1) acute lymphocytic leukemia (ALL), the most common leukemia, which starts in the lymphoid cells of the bone marrow; (2) acute myelogenous leukemia (AML), also sometimes called acute granulocytic leukemia, acute melocytic leukemia, acute myeloid lleukemia and acute non-lymphocytic leukemia, which starts in the myeloid cells of the bone marrow; (3) chronic lymphocytic leukemia (CLL), a slow-growing leukemia that starts in the lymphoid cells of the bone marrow; (4) chronic myelogenous leukemia (CML), also called chronic myeloid leukemia, which starts in the myeloid cells of the bone marrow; (5) myeloproliferative neoplasms, which are a group of blood disorders, and chronic myelomonocytic leukemia (MPNs), which starts in the myeloid cells of the bone marrow, which can lead to AML. The causes of leukemia in pediatric patients remain elusive. Several genetic syndromes and immune disorders are associated with both ALL and AML risk, although most cases are not familial. These include Down syndrome (DS), Li-Fraumeni syndrome, neurofibromatosis, DNA repair deficiency syndromes like Fanconi anemia and Bloom syndrome, and rare inherited bone marrow failure syndromes like Kostmann syndrome, Diamond-Blackfan anemia, dyskeratosis congenita, and Schwachman-Diamond syndrome [Bispo, J A B., et al. Cold Spring Harb. Perspect. Med. (2020) 10 (6): a034819].

The term “lymphocyte” refers to a small white blood cell formed in lymphatic tissue throughout the body and in normal adults making up about 22-28% of the total number of leukocytes in the circulating blood that plays a large role in defending the body against disease. Individual lymphocytes are specialized in that they are committed to respond to a limited set of structurally related antigens. This commitment, which exists before the first contact of the immune system with a given antigen, is expressed by the presence on the lymphocyte's surface membrane of receptors specific for determinants (epitopes) on the antigen. Each lymphocyte possesses a population of receptors, all of which have identical combining sites. One set, or clone, of lymphocytes differs from another clone in the structure of the combining region of its receptors and thus differs in the epitopes that it can recognize. Lymphocytes differ from each other not only in the specificity of their receptors, but also in their functions. Lymphocytes are much more common in the lymphatic system, and include B cells, T cells, natural killer T (NKT) cells, and natural killer (NK) cells. There are two broad categories of lymphocytes, namely T cells and B cells. T-cells are responsible for cell-mediated immunity whereas B-cells are responsible for humoral immunity (relating to antibodies). T-cells are so-named such because these lymphocytes mature in the thymus; B-cells mature in bone marrow. B cells make antibodies that bind to pathogens to enable their destruction. CD4+ (helper) T cells coordinate the immune response. CD8+ (cytotoxic) T cells, NKT, and Natural Killer (NK) cells are able to kill cells of the body that are, e.g., infected by a virus or display an antigenic sequence.

The term “lymphocyte activation” or “activation” refers to stimulation of lymphocytes by specific antigens, nonspecific mitogens, or allogeneic cells resulting in synthesis of RNA, protein and DNA and production of lymphokines, the soluble product of lymphocytes; it is followed by proliferation and differentiation of various effector and memory cells. For example, a mature B cell can be activated by an encounter with an antigen that expresses epitopes that are recognized by its cell surface immunoglobulin (Ig). The activation process may be a direct one, dependent on cross-linkage of membrane Ig molecules by the antigen (cross-linkage-dependent B cell activation) or an indirect one, occurring most efficiently in the context of an intimate interaction with a helper T cell (“cognate help process”). T-cell activation is dependent on the interaction of the TCR/CD3 complex with its cognate ligand, a peptide bound in the groove of a class I or class II MHC molecule. The molecular events set in motion by receptor engagement are complex. Full responsiveness of a T cell requires, in addition to receptor engagement, an accessory cell-delivered costimulatory activity, e.g., engagement of CD28 on the T cell by CD80 and/or CD86 on the antigen presenting cell (APC).

The term “lymphoma” as used herein refers to a malignant solid neoplasm of the lymphoid system, which produces immune cells. Abnormal lymphocytes become lymphoma cells, which multiply and collect in the lymph nodes. Over time, these cancerous cells impair the immune system. There are two categories of lymphomas: Hodgkin lymphoma and non-Hodgkin lymphoma. About 12 percent of people with lymphoma have Hodgkin lymphoma. Most non-Hodgkin lymphomas are B-cell lymphomas, and either grow quickly (high-grade) or slowly (low-grade). There are over a dozen types of B-cell non-Hodgkin lymphomas. The rest are T-cell lymphomas.

The term “macrophage” as used herein refers to a mononuclear, actively phagocytic cell arising from monocytic stem cells in the bone marrow. These cells are widely distributed in the body and vary in morphology and motility. Phagocytic activity is typically mediated by serum recognition factors, including certain immunoglobulins and components of the complement system, but also may be nonspecific. Macrophages also are involved in both the production of antibodies and in cell-mediated immune responses, particularly in presenting antigens to lymphocytes. They secrete a variety of immunoregulatory molecules. Polarization of immune effector cells is exemplified by the induced differentiation of macrophages into M1 and M2 macrophages during type 1 and type 2 immune responses, respectively [Ma, Q., Frontiers in Immunol. (2020) 11: 1060, citing Biswas, S K and Mantovani, A. Nat. Immunol. (2010) 11: 889-96; Murray, P J and Wynn, TA. Nat. Rev. Immunol. (2011) 11: 723-37; Hussell, T. and Bell, TJ. Nat. Rev. Immunol. (2014) 14: 81-93; Murray, P J., et al. Immunity (2014) 41: 14-20]. M2 cells can be further separated into distinctive 2a, 2b, 2c, and 2d subgroups according to their activating signals, secreted cytokines, and activities. IL-4 and IL-13 are major inducers of M2a polarization. M2a cells regulate tissue repair and the internalization of proinflammatory molecules by upregulating the expression of arginase-1 (ARG1), mannose receptor C-type 1 (MRC1, CD206), major histocompatibility complex (MHC) II, and IL-10 and TGF-β. M2b cells are activated by immune complexes or lipopolysaccharides (LPS) and produce IL-1, IL-6, IL-10, and TNF-α to activate Th2 cells and anti-inflammatory activities. M2c cells are activated in response to IL-10, TGF-β and glucocorticoids. M2cs produce IL-10 and TGF-β to suppress inflammatory responses. M2ds, which are activated by IL-6 and adenosine, are associated with tumor microenvironment and, hence, are named tumor-associated macrophages (TAMs). Polarization of TAMs is regulated by cell signaling molecules, such as IL-4 and IFN-γ, as well as extracellular metabolites, such as lactate, in the tumor microenvironment [Hobson-Gutierrez, SA and Carmona-Fontaine, C. Dis. Model Mech. (2018) 11: dmm034462.10.1242/dmm.034462]. Polarization of macrophages exhibits considerable plasticity with regard to their cell source, inducing signal, mechanism of differentiation, and interconversion between subtypes [Id., citing Biswas, S K and Mantovani, A. Nat. Immunol. (2010) 11: 889-96; Murray, P J and Wynn, TA. Nat. Rev. Immunol. (2011) 11: 723-37; Hussell, T. and Bell, TJ. Nat. Rev. Immunol. (2014) 14: 81-93; Murray, P J et al. Immunity (2014) 41: 14-20]

The term “maintenance therapy” or “continuous therapy” as used herein refers to the ongoing treatment of cancer after it has responded to induction therapy to prevent relapse.

The terms “Major Histocompatibility Complex (MHC), MHC-like molecule” and “HLA” are used interchangeably herein to refer to cell-surface molecules that display a molecular fraction known as an epitope or an antigen and mediate interactions of leukocytes with other leukocyte or body cells. MHCs are encoded by a large gene group and can be organized into three subgroups-class I, class II, and class III. In humans, the MHC gene complex is called HLA (“Human leukocyte antigen”); in mice, it is called H-2 (for “histocompatibility”). Both species have three main MHC class I genes, which are called HLA-A, HLA-B, and HLA-C in humans, and H2-K, H2-D and H2-L in the mouse. These encode the α chain of the respective MHC class I proteins. The other subunit of an MHC class I molecule is β2-microglobulin. The class II region includes the genes for the α and β chains (designated A and B) of the MHC class II molecules HLA-DR, HLA-DP, and HLA-DQ in humans. Also in the MHC class II region are the genes for the TAP1:TAP2 peptide transporter, the PSMB (or LMP) genes that encode proteasome subunits, the genes encoding the DMa and BMP chains (DMA and DMB), the genes encoding the α and β chains of the DO molecule (DOA and DOB, respectively), and the gene encoding tapasin (TAPBP). The class II genes encode various other proteins with functions in immunity. The DMA and DMB genes encoding the subunits of the HLA-DM molecule that catalyzes peptide binding to MHC class II molecules are related to the MHC class II genes, as are the DOA and DOB genes that encode the subunits of the regulatory HLA-DO molecule. [Janeway's Immunobiology. 9th ed., GS, Garland Science, Taylor & Francis Group, 2017. pps. 232-233]. In humans, there are three MHC class II isotypes: HLA-DR, HLA-DP, and HLA-DQ, encoded by α and β chain genes within the Human Leukocyte Antigen (HLA) locus on chromosome 6 [Wosen, J E., et al. Front. Immunol. (2018) doi.10.3389/fimmu.2018.02144].

The term “MHC restriction” as used herein refers to the requirement that APCs or target cells express MHC molecules that a T cell recognizes as self in order for T cell to respond to the antigen presented by that APC or target cell (T cells will only recognize antigens presented by their own MHC molecules). For example, CD8 T cells bind class I MHC which are expressed on most cells in the body, and CD4 T cells bind class II MHC which are only expressed on specialized APCs.

As used herein, the terms “marker” or “cell surface marker” are used interchangeably herein to refer to an antigenic determinant or epitope found on the surface of a specific type of cell. Cell surface markers can facilitate the characterization of a cell type, its identification, and eventually its isolation. Cell sorting techniques are based on cellular biomarkers where a cell surface marker(s) may be used for either positive selection or negative selection, i.e., for inclusion or exclusion, from a cell population.

The term “memory cells” as used herein refers to B and T lymphocytes generated during a primary immune response that remain in a quiescent state until fully activated by a subsequent exposure to specific antigen (secondary immune response). Naïve T cells and uncommitted, resting memory T cells produce principally IL-2, which they use for their own growth. Memory cells generally are more sensitive than naïve lymphocytes to antigen and respond rapidly on reexposure to the antigen that originally induced them. During an immune response, naïve T cells (T_{N}) are primed by antigen-presenting cells (APCs). Depending on the strength and quality of stimulatory signals, proliferating T cells progress along a differentiation pathway that culminates in the generation of terminally differentiated short-lived effector T (T_{EFF}) cells. When antigenic and inflammatory stimuli cease, primed T cells become quiescent and enter into the memory stem cell (T_{SCM}), central memory (T_{CM}) cell or effector memory (T_{EM}) cell pools, depending on the signal strength received. T_{SCM} cells possess stem cell-like attributes to a greater extent than any other memory lymphocyte population. Although both T_{CM} and T_{EM} cells can also undergo self-renewal, the capacity to form diverse progeny is progressively restricted, so that only T_{SCM} cells are capable of generating all three memory subsets and T_{EFF} cells; T_{CM} cells can give rise to T_{CM}, T_{EM} and T_{EFF} cells, and T_{EM} cells can only produce themselves and T_{EFF} cells. [Gattinoni, L., et al. Nature Revs. Cancer 12 (2012) 671-84]. Memory T cells express a CD62L^{lo}CD44^{hi} phenotype. [Gerberick, G F., et al. Toxicol. Appl. Pharmacol. (1997) 146 (1): 1-10].

The term “modulate” as used herein means to regulate, alter, adapt, or adjust to a certain measure or proportion. Such modulation may be any change, including an undetectable change.

As used herein, the term “mutation” refers to a change of the DNA sequence within a gene or chromosome of an organism resulting in the creation of a new character or trait not found in the parental type, or the process by which such a change occurs in a chromosome, either through an alteration in the nucleotide sequence of the DNA coding for a gene or through a change in the physical arrangement of a chromosome. Three mechanisms of mutation include substitution (exchange of one base pair for another), addition (the insertion of one or more bases into a sequence), and deletion (loss of one or more base pairs).

The term “myeloproliferative neoplasms” as used herein refers to a group of blood disorders in which the bone marrow produces too many WBCs, RBCs or platelets. There are 6 types, characterized by which blood cells are abnormal: chronic eosinophilic leukemia; chronic myelogenous leukemia; chronic neutrofilic leukemia; essential thrombocythemia; polycythemia vera (too many RBCs); and primary myelofibrosis, or chronic idiopathic myelofibrosis (meaning abnormal buildup of blood cells in the bone marrow).

The term “naïve T cell” (also referred to as virgin, inexperienced or unprimed cells) as used herein refers to T cells and B cells that have generated an antigen receptor (TCR for T cells, BCR for B cells) of a particular specificity, but have never encountered the antigen. For example, before helper T cells and B cells can interact to produce specific antibody, the antigen-specific T cell precursors must be primed. Naïve T cells produce principally IL-2, which they use for their own growth. Naïve T cells are conventionally defined by coexpression of the RA isoform of the transmembrane phosphatase CD45, the lymph node homing molecules L-selectin (CD62L) and CCR7, and the costimulatory receptors CD27 and CD28. [De Rosa, S C et al. Nature Med. (2001) 7: 245-48]. Naïve cells express a CD62L^{hi}CD44^{lo} phenotype. [Gerberick, G F., et al. Toxicol. Appl. Pharmacol. (1997) 146 (1): 1-10].

The term “NOTCH” as used herein refers to the receptor in a highly conserved pleiotropic signaling pathway. NOTCH signaling plays fundamental roles in cell differentiation, proliferation and apoptosis across all species. Canonically, a cell membrane-tethered NOTCH ligand binds to NOTCH receptor on a neighboring cell, which induces enzymatic cleavages of the NOTCH receptor. In mammals, there are five NOTCH ligands (Dll-1, Dll-4, Jag-1, and Jag-2 are activators, and Dll-3 is an inhibitor) and four NOTCH receptors (Notch-1, Notch-2, Notch-3, and Notch-4), all of which contain extracellular epidermal growth factor (EGF)-like domains executing ligand-receptor binding.

After binding to its ligand, the NOTCH receptor undergoes two successive enzymatic cleavages mediated by ADAM10 and 7-secretase, releasing the NICD into cell nucleus where it binds with NOTCH signaling transcription factor CSL together with other co-factors to activate gene transcription [Shen, W., et al. Front. Cell Dev. Biol. (2021) 9: doi.org/10.3389/fcell.2021.652273, citing Kopan, R. and Llagan, MA Cell (2009) 137: 216-33]. The most conserved direct targets of NOTCH signaling are basic helix-loop-helix (bHLH) transcription factors of hairy/enhancer of split (Hes) family and hairy/enhancer of split related with YRPW motif (Hey) family [Id., citing Iso, T., et al. J. Cell Physiol. (2003) 194: 237-55; Borggrefe, T. and Oswald, F. Cell Mol. Life Sci. (2009) 66: 1631-46]. The released Notch intracellular domain (NICD) subsequently migrates into the cell nucleus, where it binds with NOTCH signaling transcriptional factor CSL (CBF-1/RBP-J in mammals, Su(H) in Drosophila, and Lag-1 in Caenorhabditis elegans) together with other transcription co-factors to activate gene transcription.

Unlike many other signaling pathways that contain kinase cascade-mediated signaling amplification processes, notwithstanding that NOTCH signaling does not contain a signaling intermediate to amplify the signal, NOTCH signaling can be activated at distinct strength levels for the following three reasons: (1) one NOTCH receptor can only release one NICD after ligand-receptor binding, (2) no signal intermediate or kinase cascade is involved to amplify the initial signal, and (3) NICD is subjected to proteasome-mediated degradation after transcriptional activation [Id., citing Fryer, C J., et al. Mol. Cell (2004) 16: 509-20]. Since CSL and other transcriptional co-factors are always readily present in cell nucleus, the level of NICD generated determines the strength and duration of NOTCH signaling. Collectively, the amount of ligand and receptor presented on the cell membrane, the type of ligand binding to receptor, glycosylation of EGF domain, and lipid-ligand interaction all influence ligand-receptor binding and consequently the amount of NICD released. Moreover, the four active NOTCH ligands (Jag-1, Jag-2, Dll-1, and Dll-4) in mammals exhibit different binding affinities [Id., citing Benedito, R., et al. Cell (2009) 137: 1124-35; Groot, A J., et al. Mole. Cell Biol. (2014) 34: 2822-32; Gama-Norton, L., et al. Nat. Commun. (2015) 6: 8510; Nandagopal, N., et al. Cell (2018) 172: 869e-880e], which further diversifies the levels of NOTCH signaling strength in different cell contexts. In addition, EGF domain glycosylation by fringe glycosyltransferase can change ligand-receptor binding affinity and facilitate NOTCH receptor cleavage [Id., citing Stanley, P. and Okajima, T. Curr. Top. Dev. Biol. (2010) 92: 131-64; Takeuchi, H. and Haltiwanger, RS. Semin. Cell Dev. Biol. (2010) 21: 638; Kakuda, S. and Haltiwanger, RS. Dev. Cell (2017) 40: 193-201].

The stability of NICD affects the duration of NOTCH signaling. NICD is generated following ligand-receptor binding and shuttles into cell nucleus, where it binds to transcription factor CSL together with co-factor of mastermind-like protein (MamL) and other chromatin modifiers to activate gene transcription. In addition to transcriptional regulations, CSL and MamL also recruit kinase CDK8 to phosphorylate NICD, which triggers protein ubiquitination on PEST (proline, glutamic acid, serine, and threonine-enriched) domain of NICD and proteasome-mediated NICD degradation [Id., citing Fryer, C J., et al. Mol. Cell (2004) 16: 509-20]. Thus, NOTCH signaling is quickly reduced without re-supply of new NICD, which is necessary to maintain proper levels of NOTCH signaling strength. In addition, NICD-CSL binding also triggers NICD ubiquitination that leads to its subsequent degradation [Id., citing Fryer, C J., et al. Mol. Cell (2004) 16: 509-20].

NOTCH signaling strength determines αβ T-cell (vs. γδ T-cell) specification in the T-cell lineage. Postnatal development of T immune cell in the thymus requires activation of NOTCH signaling in the hematopoietic progenitor cells (HPCs) that migrated from the bone marrow. NOTCH signaling activation inhibits non-T-cell cells including myeloid lineage during early stages and B-cell during late stages [Id., citing Wilson, A. et al. J. Exp. Med. (2001) 194: 1003-12]. However, after T-cell fate is committed, the strength of NOTCH in T-cell lineage determines T-cell sub-lineage specifications between αβ T-cell and γδ T-cell. An in vitro study showed that human OP9-Dll1/4 that served as NOTCH signal-sending cell can stimulate the differentiation of human hematopoietic progenitor cells (HPCs) into T-cells populated with both αβ T-cell and γδ T-cell. Interestingly, lowering NOTCH signaling strength via adding a series of γ-secretase inhibitor (DAPT) with increasing concentrations gradually switched γδ T-cell into αβ T-cell [Id., citing Van de Walle, I., et al., J. Exp. Med. (2013) 210: 683-97], documenting NOTCH-strength-dependent cell fate determination between the two T-cell subtypes.

The strength changes of NOTCH signaling in the T-cell lineage are caused by binding with different NOTCH ligands that hold distinct receptor binding affinities. In human HPCs, Jag-2 exhibited strong NOTCH activation potential and directed HPCs predominantly into γδ T-cell [Id., citing Van de Walle, I., et al. Blood (2011) 117: 4449-59; Van de Walle, I., et al., J. Exp. Med. (2013) 210: 683-97]; Dll-4 induced relatively weak NOTCH signaling and generated both γδ T-cell and αβ T-cells, while Jag-1 induced the weakest NOTCH signaling and generated mainly αβ T-cells [Id., citing Van de Walle, I., et al., J. Exp. Med. (2013) 210: 683-97]. Collectively, the diverted expression of NOTCH ligands maintained a diverse range of NOTCH signaling strength, which balances the populations of αβ T-cell and γδ T-cells. Mouse HPCs also utilize the strength difference of NOTCH signaling to determine the αβ T-cell and γδ T-cell fate, but in an opposite way so that low NOTCH signaling favors γδ T-cells (Id., citing Washburn, T., et al. Cell (1997) 88: 833-43).

The term “nuclear factor of activated T cells proteins” or “NFAT proteins” refers to a family of transcription factors whose activation is controlled by calcineurin, a calcium dependent phosphatase. During periods of sustained elevations of calcium, calcineurin dephosphorylates NFATC1-C4, allowing NFAT to translocate to the nucleus. This nuclear translocation is blocked by cyclosporine A (CSA), which blocks calcineurin activity. Once in the nucleus, NFAT binds to consensus DNA sites and controls gene transcription. Originally identified in T cells as inducers of cytokine gene expression, NFAT proteins play varied roles in cells outside of the immune system. [Horsley, V. and Pavlath, GK. J. Cell Biol. (2002) 156 (5): 771-4].

The abbreviation “NFκB” as used herein refers to a proinflammatory transcription factor that switches on multiple inflammatory genes, including cytokines, chemokines, proteases, and inhibitors of apoptosis, resulting in amplification of the inflammatory response [Barnes, PJ, (2016) Pharmacol. Rev. 68: 788-815]. The molecular pathways involved in NF-κB activation include several kinases. The classic (canonical) pathway for inflammatory stimuli and infections to activate NF-κB signaling involves the IKK (inhibitor of KB kinase) complex, which is composed of two catalytic subunits, IKK-α and IKK-β, and a regulatory subunit IKK-7 (or NFκB essential modulator [Id., citing Hayden, MS and Ghosh, S (2012) Genes Dev. 26: 203-234]. The IKK complex phosphorylates Nf-κB-bound IκBs, targeting them for degradation by the proteasome and thereby releasing NF-κB dimers that are composed of p65 and p50 subunits, which translocate to the nucleus where they bind to KB recognition sites in the promoter regions of inflammatory and immune genes, resulting in their transcriptional activation. This response depends mainly on the catalytic subunit IKK-β (also known as IKK2), which carries out IκB phosphorylation. The noncanonical (alternative) pathway involves the upstream kinase NF-κB-inducing kinase (NIK) that phosphorylates IKK-α homodimers and releases RelB and processes p100 to p52 in response to certain members of the TNF family, such as lymphotoxin-β [Id., citing Sun, SC. (2012) Immunol. Rev. 246: 125-140]. This pathway switches on different gene sets and may mediate different immune functions from the canonical pathway. Dominant-negative IKK-β inhibits most of the proinflammatory functions of NF-κB, whereas inhibiting IKK-α has a role only in response to limited stimuli and in certain cells, such as B-lymphocytes. The noncanonical pathway is involved in development of the immune system and in adaptive immune responses. The coactivator molecule CD40, which is expressed on antigen-presenting cells, such as dendritic cells and macrophages, activates the noncanonical pathway when it interacts with CD40L expressed on lymphocytes [Id., citing Lombardi, V et al. (2010) Int. Arch. Allergy Immunol. 151: 179-89].

The term “overall survival” as used herein refers to the length of time from either the date of diagnosis or the start of treatment for a disease, such as cancer, that subjects diagnosed with the disease are still alive.

The term “PD-1” or “programmed cell death protein 1” as used herein refers to an inhibitory receptor expressed on the surface of activated T cells. Its ligands, PD-L1 and PD-L2, are expressed on the surface of DCs or macrophages. PD-1 and its ligands PD-L1/PL-L2 act as co-inhibitory factors that can limit the development of the T cell response. PD-L1 is overexpressed on tumor cells or on non-transformed cells in the tumor microenvironment [Pardoll, DM. Nat. Rev. Cancer (2012) 12: 252-64]. PD-L1 expressed on the tumor cells binds to PD-1 receptors on the activated T cells, which leads to the inhibition of the cytotoxic T cells. These deactivated T cells remain inhibited in the tumor microenvironment.

The term “peptide” is used herein to designate a series of amino acid residues, connected one to the other typically by peptide bonds between the alpha-amino and carbonyl groups of the adjacent amino acids. Peptides are typically 9 amino acids in length, but can be as short as 8 amino acids in length, and as long as 14 amino acids in length. A series of amino acids are considered an “oligopeptide” when the amino acid length is greater than about 14 amino acids in length, typically up to about 30 to 40 residues in length. When the amino acid residue length exceeds 40 amino acid residues, the series of amino acid residues is termed a “polypeptide”.

The terms “peripheral blood mononuclear cells” or “PBMCs” are used interchangeably herein to refer to blood cells having a single round nucleus such as, for example, a lymphocyte or a monocyte. PBMCs are a critical component in the immune system's responses to infections.

The terms “peripheral lymphoid organ”, “peripheral lymphoid tissue”, and “secondary lymphoid tissue” as used herein refer to the lymph nodes, spleen, and mucosa-associated lymphoid tissues in which adaptive immune responses are induced.

The term “pharmaceutical composition” is used herein to refer to a composition that is employed to prevent, reduce in intensity, cure or otherwise treat a target condition or disease.

The term “phenotype” as used herein refers to qualitative and quantitative observable characteristics of cells. A cell's phenotype is the culmination of several cellular processes through a complex network of molecular interactions that ultimately result in a unique morphological signature. Clinical, biochemical and imaging methodologies can be used to refine and characterize a phenotype.

The term “polarization of immune cells” as used herein refers to a process in which immune cells adopt distinct programs and perform specialized functions in response to specific signals. For example, Naïve CD4+T, i.e., Th0, cells can differentiate into TH1, TH2, or TH17 cells in response to specific stimulating signals. Polarized Th cells play critical roles in the initiation, amplification and resolution or progression of Type 1, type 2, or type 3 immunity. Similarly, ILCs can polarize into ILC1, ILC2, and ILC3 subpopulations to regulate associated immune responses. [Ma, Q. Front. Immunol. (2020) 11: 1060].

The term “polyfunctional” and its various grammatical forms as used herein refer to the ability of individual T cells to produce multiple pro-inflammatory cytokines (e.g., IFN-γ, IL-2 and TNF) in response to activation [Boyd., A., et al PLoS One (2015) 10 (6): e0128714]. Progressive loss of polyfunctionality and concomitant upregulation of inhibitory receptor(s) expression are hallmarks of T cell exhaustion. Five inhibitory receptors (PD-1, LAG-3, TIM-3, TIGIT and BTLA) are well documented in negative regulation of T cell function. [Odorizzi, P M and Wherry, EJ. J. Immunol. (2012) 188 (7): 2957-65]. Consequently, polyfunctionality is routinely assessed to measure the T cell response or to determine the quality of that response.

The term “precursors of exhausted T cells” or “T_{PEX} cells” as used herein refers to a population of exhausted T cells with precursor characteristics that self-renew that express molecules typically associated with memory T cells, such as the transcription factor TCF1. [Utzschneider, C T et al. Nature Immunology (2020) 21: 1256-66]. In contrast to most exhausted T cells, TCF1⁺ cells lack expression of TIM-3, retain high proliferative potential and undergo long-term self-renewal, while also replenishing the dominant population of TCF1—exhausted effector T cells. [Id., citing Kalllies, A., et al. Nat. Rev. Immunol. (2020) 20: 128-36; Wu, T., et al. Sci. Immunol. (2016) 1: eaai8593; Utzschneider, D., et al. Immunity (2016) 45: 415-27; Im, S. J., et al. Nature (2016) 537: 417-21; He, R., et al. Nature (2016) 537: 412-428; Leong, Y A., et al. Nat. Immunol. (2016) 17: 1187-96]. T., cells are therefore essential to perpetuate antigen-specific T cell responses over long periods. [Id., citing McLane, L M et al. Annu Rev. Immunol. (2019) 37: 457-95; Hashimoto, M., et al. Annu Rev. Med. (2018) 69: 301-18; Lugli, E., et al. Trends Immunol. (2020) 41: 17-28; Kallies, A., et al. Nat. Rev. Immunol. (2020) 20: 128-36]. Despite exhibiting critical features of T cell exhaustion, such as high expression of TOX and PD-1, as well as an impaired ability to produce cytokines, TCF1+T_{PEX} cells in chronic infection retain the highest developmental potential among the exhausted T cell pool. [Id., citing Kallies, A., et al. Nat. Rev. Immunol. (2020) 10: 128-36; Alfei, F., et al. Nature (2019) 571: 265-69; Khan, O., et al. Nature (2019) 571: 211-18; Yao, C., et al. Nat. Imunol. (2019) 20: 890-901; Scott, A C., et al. Nature (2019) 571: 270-74; Seo, H., et al. Proc. Natl Acad. Sci. USA (2019) 116: 12410-15; Utzschneider, D T., et al. Immunity (2016) 45: 415-27; Hudson, W H., et al. Immunity (2019) 51: 1043-58; Zander, R., et al. Immunity (2019) 51: 1028-42].

TCF1-CX3CR1+ exhausted T cells are the immediate progeny of TCF1⁺ T_{PEX} cells, maintain the highest effector function, and are critical for mediating some level of control in chronic viral infections. [Id. citing Hudson, W H., et al. Immunity (2019) 51: 1043-58; Zander, R., et al. Immunity (2019) 51: 1028-42]. Loss of CX3CR1 demarcates acquisition of a terminally exhausted state in which effector functions are strongly diminished. [Id. citing Hudson, W H et al. Immunity (2019) 51: 1043-58; Zander, R. et al. Immunity (2019) 51: 1028-42].

The term “priming” as used herein refers to the first encounter with a given antigen, which generates a primary adaptive immune response. Priming involves several steps: antigen uptake, processing, and cell surface expression bound to class II MHC molecules by an antigen presenting cell, recirculation and antigen-specific trapping of helper T cell precursors in lymphoid tissue, and T cell proliferation and differentiation. [Janeway, CA, Jr., “The priming of helper T cells, Semin. Immunol. (1989) 1(1): 13-20]. Helper T cells express CD4, but not all CD4 T cells are helper cells. Id. The signals required for clonal expansion of helper T cells differ from those required by other CD4 T cells. The critical antigen-presenting cell for helper T cell priming appears to be a macrophage; and the critical second signal for helper T cell growth is the macrophage product interleukin 1 (IL-1). Id. If the primed T cells and/or B cells receive a second, co-stimulatory signal, they become activated T cells or B cells.

“The term “progenitor cell” as used herein refers to an early descendant of a stem cell that can only differentiate, but can no longer renew itself. Progenitor cells mature into precursor cells that mature into mature phenotypes.

The term “progenitor or stem-like exhausted CD8⁺ T cells” as used herein is defined by intermediate expression of PD-1 and expression of the chemokine receptor CXCR5. [Miller, B C et al. Nat. Immunol. (2019) 20 (11): 1556].

The term “progression” as used herein refers to the course of disease as it becomes worse or spreads in the body.

The term “progression-free survival” or PFS” as used herein refers to the length of time during and after the treatment of the disease that a patient lives with the disease but it does not get worse.

The term “proliferate” and its various grammatical forms as used herein is meant to refer to the process that results in an increase of the number of cells, and is defined by the balance between cell division and cell loss through cell death or differentiation.

The term “purified” and its various grammatical forms as used herein refers to being free from extraneous or undesirable elements.

The term “recurrent cancer” or “recurrence” means a cancer that has come back, usually after a period of time during which the cancer could not be detected. The cancer may come back to the same place as the primary tumor or to another place in the body.

The term “refractory cancer” or “resistant cancer” means a cancer that does not respond to treatment. The cancer may be resistant at the beginning of treatment or it may become resistant during treatment.

The term “relapse” refers to the return of a disease or the signs and symptoms of a disease after a period of improvement.

The terms “relapse-free survival” (RFS) or “disease-free survival” (DFS) mean the length of time after primary treatment for a cancer ends that the patient survives without any signs or symptoms of that cancer.

The term “RNA-seq.” as used herein refers to a process of creating short sequencing reads from RNA molecules. The steps consist of first converting the RNA into cDNA; then (optionally) amplifying the cDNA by PCR; and finally fragmenting the cDNA into short pieces/fragments. After the sequencing library is prepared, the fragments are used as input for next-generation sequencing. The resulting sequence reads contained in FASTQ files are then aligned to a known reference sequence. [Deshpande, D., et al. Frontiers Genetics (2023) 14: 997383].

The term “scFv” as used herein refers to a class of engineered functional antibodies generated by the fusion of the variable heavy (V_{H}) and variable light (V_{H}) domains of an immunoglobulin through a short polypeptide linker.

SMADS and SMAD signaling. The SMAD pathway regulates the production of IgA by B cells, maintains the protective mucosal barrier and promotes the balanced differentiation of CD4+ T cells into inflammatory T helper type 17 cells and suppressive FOXP3+T regulatory cells in the gut. To maintain intestinal integrity, the epithelial cells, myeloid cells, and lymphocytes that inhabit the gut secrete TGF β, which acts in both paracrine and autocrine fashion to activate its signal transducers, the SMAD transcription factors. The SMAD transcription factors achieve complementary context-dependent functions of TGF-β by cooperation of SMAD proteins with distinct dominant transcription activators and accessory chromatin modifiers.

Transforming growth factor-β signals through type I and II transmembrane serine/threonine protein kinase receptors. The binding of the ligand activates the type II receptors, which recruit type I receptors, forming a heterotetrameric complex. The activated type I receptors interact with and phosphorylate SMAD proteins to transduce signals. [Malhotra, N. and Kang, J. Immunology (2013) 139: 1-10, citing Li, MO and Flavell, RA. Cell (2008) 134: 392-404]. SMADs are divided into three functional classes: receptor activated (R-SMADs), common mediator (Co-SMADs), and inhibitory (I-SMADs). The R-SMADs are phosphorylated by the activated Type I receptors on their C-terminus. This group includes SMAD1, 2, 3, 5 and 8. [Id., citing Derynck, R., et al. Cell (1998) 95: 737-40] Phosphorylated R-SMADs form homotrimers and interact with SMAD4, a Co-SMAD that mediates translocation of R-SMADs into the nucleus. R-SMADs and Co-SMAD complexes function as TFs and regulate the expression of genes associated with cell proliferation, survival and differentiation. Their activity is counter-regulated by the I-SMADs, SMAD6/7, which interact with type I receptors and competitively interfere with the activation of R-SMADs. [Id., citing Schmierer, B. and Hill, CS. Nat. Rev. Mol. Cell Biol. (2007) 8: 970-82]. SMADs constantly shuttle between the cytoplasm and nucleus but they are retained longer in the nucleus upon activation by TGFβRI.

A number of accessory proteins have been identified that mediate the interactions of R-SMADs with their membrane receptors. Examples of such proteins are Disabled-2 (Dab2) and SMAD anchor for receptor activation (SARA, encoded by Zfyve9). Dab2 interacts with SMAD2/3 and promotes their association with TGFβRI and RII. [Id., citing Hocevar, B A et al. EMBO J. (2001) 20: 2789-801] In the immune system Dab2 is required for normal Treg cell function, perhaps in settings that require high TGF-β signalling. [Id., citing Jain, N., et al. J. Immunol. (2009) 183: 4192-6] SARA tethers unphosphorylated SMADs to the TGFβRI kinase in the cytoplasm and dissociates from them upon SMAD activation. [Id., citing Brown, K A, et al. J. Cell Biochem. (2007) 101: 9-33].

Due to their weak binding, SMADs work as oligomeric complexes at the SMAD binding element and require synergistic actions from other TFs. Unlike some TFs that can bind to relatively unoccupied DNA and directly recruit transcription activation complexes to proximal promoters, SMAD TFs are dependent on chromatin modifiers to assemble the basal transcription machinery. [Id., citing Ross, S., et al. EMBO J. (2006) 25: 4490-502]. Once positioned on the chromatin, SMADs promote further remodelling by recruiting histone-modifying enzymes such as the histone acetylase p300 (which acetylates histone H3), the SWI/SNF component Brg1, and the histone demethylase KDM6B (JMJD3), [Id., citing Ross, S., et al. EMBO J. (2006) 25: 4490-502; Kim, S W., et al. Dev. Biol. (2011) 357: 492-504] or by interacting with TFs that can modulate the activities of chromatin regulators, such as ATF-3 [Id., citing Kang, Y., et al. Mol. Cell (2003) 11: 915-26], HEB/E2A [Yoon, S J., et al. Genes Dev. (2011) 25: 1654-61] and LEF1 [Id., citing Labbe, E., et al. Proc. Natl Acad. Sci. USA (2000) 97: 8358-63]. Furthermore, factors that discriminate histone modification marks can interact with SMAD2/3 and distribute them to discrete chromatin regions. One example of this mode of shuttling involves TRIM33 (Tif1γ), which selectively binds to active H3 modifications, [Id., citing Agricola, E. et al. Mol. Cell (2011) 43: 85-96; Xi, Q. et al. Cell (2011) 147: 1511-24]. TRIM33 binds to SMAD2/3 and shuttles them to specific promoters for chromatin remodeling and transcription activation. [Id., citing Xi, Q., et al. Cell (2011) 147: 1511-24; He, W., et al. Cell (2006)].

The co-operation of chromatin modifiers and TFs in regulating gene transcription underpins the context-dependent function of TGF-β-activated SMADs. System-wide studies to map SMAD docking sites in the genome of diverse cell lineages showed that SMADs (including SMAD3 activation downstream of TGF-β) are co-localized with, and are regulated by, cell-type-specific master TFs, [Id., citing Mizutani, A., et al. J. boil. Chem. (2011) 286: 29848-60; Mullen, A C., et al. Cell (2011) 147: 565-76; Trompouki, E., et al. Cell (2011) 147: 577-89]. Hence, a substantial proportion of, if not most, SMAD occupancy and transcriptional modulation in a cell type reflects the global positioning of master TFs and their gene network. The master TFs help in promoting SMAD binding by establishing open chromatin, where SMADs bind to SMAD binding element and form a physical complex with the master TFs. The chromatin docking of SMADs (directed by cell-type-specific master TFs) enables them to mediate expression of a wide variety of unrelated genes in distinct cell types, [Id., citing Mullen, A C., et al. Cell (2011) 147: 565-76; Trompouki, E., et al. Cell (2011) 147: 577-89]. The global SMAD genomic occupancy is also likely to evolve with the lymphocyte activational state because the two major activation induced TFs, nuclear factor of activated T cells (NFAT) and activator protein 1 (AP-1), co-operatively control genes that dominantly specify cell fate, such as FOXP3,34,35 and cell survival. [Id., citing Zhang, Y., et al. Nature (1998) 394: 909-13; Verrechhia, F., et al. Oncogene (2001) 20: 2205-11; Yamamura, Y., et al. J. Biol. Chem. (2000) 275: 36295-302].

The term “Signal transducer and activator of transcription 3” or “STAT3”) as used herein refers to a transcription factor that is activated downstream of a large range of cell surface receptors. It forms part of a family of proteins that also includes STAT1, 2, 4, 5A, 5B, and 6, which are activated in a similar manner downstream of surface receptors. Binding of their ligand by these receptors, leads to the activation of receptor-associated Janus activating kinases (JAKs). The activated JAKs then phosphorylate the receptor providing docking sites for STATs, which in turn become tyrosine phosphorylated. This leads to the formation of homodimers or heterodimers, followed by translocation to the nucleus where the dimers bind to DNA and induce transcription of a broad range of target genes [Deenick, E K., et al. Front. Immunol. (2018) 9: doi.org/10.3389/fimmu.2018.00168, O'Shea, J J., et al. N. Engl. J. Med. (2013) 368 (20): 161-70; Shuai, K. and Liu, B. Nat. Rev. Immunol. (2003) 3 (11): 900-11]. Many of the cytokine receptors that lead to STAT3 activation are expressed by lymphocytes including those for IL-6, IL-10, IL-21, IL-23, and IFNs. STAT3 plays a central role in regulation of immune responses. Loss of function (LOF) mutations in STAT3 cause the primary immunodeficiency autosomal dominant hyper IgE syndrome (ADHIES), which is characterized by defects in both T and B cells [Id., citing Holland, S M., et al. N. Engl. J. Med. (2007) 357 (16): 1608-19; Minegishi, Y., et al. Nature (2007) 448 (7157): 1058-62]. Gain of function (GOF) mutations in STAT3 have been identified in patients who presented with early onset autoimmunity as well as immunodeficiency [Id., citing Flanagan, S E., et al. Nat. Genet. (2014) 46 (8): 812-4; Haapaniemi, E M et al. Blood (2015) 125 (4): 639-48; Milner, J D., et al. Blood (2015) 125 (40: 591-9].

B cell responses. Multiple findings in patients with dysregulated STAT3 function point to a role for STAT3 in regulating human B cells responses. For example, although patients with STAT3LOF mutations have relatively normal levels of total serum IgM, IgG, and IgA, they have elevated levels of serum IgE, defects in antigen specific antibody responses and reduced memory B cells [Id., citing Chandesris, M-O., et al. Medicine (Baltimore) 2012]91 (4): el-19; Deeskin, SC., et al. J. Clin. Invest. (1985) 75 (1): 26-34; Leung, D Y., et al. J. Allergy Clin. Immunol. (1988) 81 (6): 1082-7; Sheerin, K A and Bckley, RH. J. Allergy Clin. Immunol. (1991) 87 (4): 803-11; Meyer-Bahlburg, A., et al. J. Allergy Clin. Immunol. (2012) 129 (2): 559-62]. Further, the STAT3-activating cytokines IL-21, and to a lesser extent IL-10, are potent B cell activators.

CD4+ T cells. Naïve CD4⁺ T cells are able differentiate into distinct effector subsets that play specific roles in the immune response. These subsets include TH1, TH2, TH9, TH17, TH22, TFH cells, and Tregs. The differentiation of CD4⁺ T cells is determined by the cytokine milieu at the time of activation and numerous STAT3-signaling cytokines have been implicated in this process [Id., citing O'Shea, J J and Paul, WE. Science (2010) 327 (5969): 1098-102; Tangye, S G., et al. Nat. Rev. Immunol. (2013) 13 (6): 412-26].

T_{H}17. The differentiation of human Th17 cells is controlled by the action of several STAT3-activating cytokines including IL-6, IL-21, and IL-23 [Id., citing Acosta-Rodriguez, E V., et al. Nat. Immunol. (2007) 8 (6): 639-46; Wilson, NJ., et al. Nat. Immunol. (2007) 8 (9): 950-7; Yang, X O., et al. Immunity (2008) 28 (1): 29-39].

T_{H}1/TH2. In contrast to TH17 cells, the generation of human TH1 and TH2 is thought to act primarily through IL-12/STAT4 and IL-4/STAT6 signaling, respectively [Id., citing O'Shea, J J and Paul, WE. Science (2010) 327 (5969): 1098-102; Tangye, S G., et al. Nat. Rev. Immunol. (2013) 13 (6): 412-26]. Consistent with this, generation of these populations was found to be largely STAT3-independent, as shown by normal frequencies of CXCR3+CCR6− and CXCR3−CCR6− and IFNγ-producing and IL-4, IL-5, IL-13-producing cells, respectively, in AD-HIES patients [Id., citing Ma, C S et al. J. Allergy Clin. Immunol. (2015) 136 (4): 993-1006; Milner, J D et al. Nature (2008) 452 (7188): 773-6; Renner, E D et al. J. Allergy Clin. Immunol. (2008) 122 (1): 181-7]. Similarly, naïve CD4⁺ T cells from STAT3LOF patients could differentiate into TH1 or TH2 cells when cultured under the relevant polarizing conditions [Id., citing Ma, C S et al. (41, 47). IFNγ expression tended to be increased in STAT3-deficient CD4⁺ T cells [Id., citing Ma, C S et al. J. Allergy Clin. Immunol. (2015) 136 (4): 993-2006; Ma, C S et al. J. Exp. Med. (2016) 213 (8): 1589-608], suggesting STAT3 signaling may inhibit TH1 cell differentiation.

TH9. Human Th9 cells develop in the presence of TGFβ and IL-4 [Id., citing Beriou, G. et al. J. Immunol. (2010) 185 (1): 46-54; Beriou, G. et al. J. Immunol. (2010) 185 (1): 46-54; Chang, H-C et al. Nat. Immunol. (2010) 11 96]: 527-34]; however, they can also be induced by the addition of TGFβ to Th17 polarizing conditions (i.e., IL-1β/IL-6/IL-21/IL-23) [Id., citing Beriou, G. et al. J. Immunol. (2010) 185 (1): 46-54], suggesting STAT3 may be involved in Th9 cell differentiation.

TFH cells. Like TH17 differentiation, the generation of human TFH cells is driven by numerous STAT3-ativating cytokines, namely IL-6, IL-12, IL-21, and IL-27. Consistent with a requirement for STAT3 to induce this differentiation program, patients with STAT3LOF have a reduction in circulating CXCR5⁺TFH cells [Id., citing Ma, CS., et al. J. Allergy Clin. Immunol. (2015) 136 (4): 993-1006; Ma, CS., et al. Blood (2012) 119 (17): 3997-4008; Mazerolles, F., et al. J. Allergy Clin. Immunol. (2013) 131 (4): 1146-56], and naïve STAT3LOF CD4⁺ T cells failed to differentiate in vitro into IL-21-producing TFH-like cells (41, 52, 54). The role of STAT3 in differentiation and/or function of T_{F}H cells has also been demonstrated in mouse studies of Stat3-deficient T cells [Id., citing Eddahri, F., et al. Blood (2009) 113 (11): 2426-33; Nurieva, RI., et al. Immunity (2008) 29 (1): 138-49; Choi, Y S., et al. J. Innunol. (2013) 190 (7): 3049-53; McIlwain, D R., et al. Eur. J. Immunol. (2015) 45 (2): 418-27; Ray, J P., et al. Immunity (2014) 40 (3): 367-77]. However, the degree to which Stat3 is required seems to be dependent on the presence of other signals such as STAT1 and type 1 IFNs as well as the site of the immune response [Id., citing Choi, Y S., et al. J. Immunol. (2013) 190 (7): 3049-53; Ray, J P., et al. Immunity (2014) 40 (3): 367-77; Wu, H., et al. Eur. J. Immunol. (2016) 46 (5): 1152-61].

Treg. STAT appears to play a complex role in the regulation of Treg responses. patients with STAT3^{GOF} mutations much more clearly demonstrate a role for STAT3 in the regulation of Tregs. As discussed above, these patients display early onset autoimmunity that is reminiscent of patients with Immune dysregulation, polyendocrinopathy, enteropathy, X-linked (IPEX) syndrome, which is caused by mutations in FoxP3 leading to a loss of Treg function [Id., citing Bacchetta, R., et al. Ann. NY Acad. Sci. (2016) 100 (2): 1-18]. These overlapping clinical phenotypes suggested that STAT3^{GOF} patients also have dysfunctional Tregs. Consistent with this, they were found to have lower percentage of FoxP3+ cells in their blood and lower CD25 expression on their Tregs [Id., citing Milner, J D., et al. Blood (2015) 125 (4): 591-9]. This is thought to be due to increased SOCS3 levels that inhibit the activation of STAT5 downstream of IL-2 [Id., citing Milner, J D., et al. Blood (2015) 125 (4): 591-9].

Studies in mice have now demonstrated that there are different populations of Tregs that seem to be specialized for inhibiting particular T-helper populations [Id., citing Josefowicz, S Z., et al. Annu. Rev. Immunol. (2012) 30: 531-64]. Thus, a population of Treg cells that express CCR6 and are specialized for suppressing TH17 cells has been described. These “Treg17” cells, like the TH17 cells they suppress, were found to be dependent on STAT3 signaling [Id., citing Chaudhry, A., et al. Science (2009) 326 (5955): 986-91]. AD-HIES patients were also shown to have decreased CCR6+ Tregs suggesting that human “Treg17” cells may also exist and be dependent on STAT3 [Id., citing Kluger, MA., et al. J. A. Soc. Nephrol. (2014) 25 (6): 1291-302].

CD8+ T cells. STAT3 activating cytokines such as IL-21 also play a role in regulating CD8+ T cells. Studies on STAT3LOF CD8+ T cells showed they had impaired induction of perforin and granzyme B in response to IL-21; however, this could be rescued by strong TCR ligation [Id., citing Ives, M L., et al. J. Allergy Clin. Immunol. (2013) 132 (2): 400-11]. In contrast, proliferation induced by IL-21 was not affected in naïve STAT3LOF CD8+ T cells [Id., citing Ives, M L., et al. J. Allergy Clin. Immunol. (2013) 132 (2): 400-11]. STAT3 deficiency, however, did result in reduced memory CD8⁺ T cells [Id., citing Siegel, A M., et al. Immunity (2011) 35 (5): 806-18; Ives, M L., et al. J. Allergy Clin. Immunol. (2013) 132 (2): 400-11]), an effect that was shown to be cell intrinsic[Id., citing Siegel, A M., et al. Immunity (2011) 35 (5): 806-18].

The term “somatic recombination” or V(D)J recombination” as used herein refers to site-specific recombination of pre-existing V, D and J gene segments in the immunoglobulin (Ig) and TCR loci to generate unique variable (V) exons. It is tightly regulated during T and B lymphocyte development to generate a highly diverse repertoire of receptors.

As used herein, the term “stimulate” and any of its grammatical forms as used herein is meant to refer to inducing activation or increasing activity.

The term “stimulate an immune cell” or “stimulating an immune cell” as used herein refers to a process (e.g., involving a signaling event or stimulus) causing or resulting in a cellular response, such as activation and/or expansion, of an immune cell, e.g. a CD8⁺ T cell.

The terms “subject” or “individual” or “patient” are used interchangeably to refer to a member of an animal species of mammalian origin, including but not limited to, mouse, rat, cat, goat, sheep, horse, hamster, ferret, pig, dog, guinea pig, rabbit and a primate, such as, for example, a monkey, ape, or human.

The phrase “subject in need thereof” as used herein refers to an eligible patient that (i) will be administered an immunotherapy according to the present disclosure, (ii) is receiving at least one immunotherapy according to the present disclosure; or (iii) has received at least one immunotherapy according to the present disclosure, unless the context and usage of the phrase indicates otherwise.

The terms “T cell” or “T lymphocyte” or are used interchangeably to refer to cells that mediate a wide range of immunologic functions, including the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on their expression of specific cell surface molecules and the secretion of cytokines. T cells recognize antigens on the surface of antigen presenting cells (APCs) and mediate their functions by interacting with, and altering, the behavior of these APCs. T cells can also be classified based on their function as helper T cells; T cells involved in inducing cellular immunity; suppressor T cells; and cytotoxic T cells. T-cell activation is dependent on the interaction of the TCR/CD3 complex with its cognate ligand, a peptide bound in the groove of a class I or class II MHC molecule. The molecular events set in motion by receptor engagement are complex. Among the earliest steps appears to be the activation of tyrosine kinases leading to the tyrosine phosphorylation of a set of substrates that control several signaling pathways. These include a set of adaptor proteins that link the TCR to the ras pathway, phospholipase Cγ1, the tyrosine phosphorylation of which increases its catalytic activity and engages the inositol phospholipid metabolic pathway, leading to elevation of intracellular free calcium concentration and activation of protein kinase C, and a series of other enzymes that control cellular growth and differentiation. Full responsiveness of a T cell requires, in addition to receptor engagement, an accessory cell-delivered costimulatory activity, e.g., engagement of CD28 on the T cell by CD80 and/or CD86 on the antigen presenting cell (APC). The ultimate amplitude and quality of the T cell immune response, which is initiated through antigen recognition by the TCR, is regulated by a balance between co-stimulatory and inhibitory signals (immune checkpoints).

Although the lineage relationship between T cell subsets remains controversial, T cells cluster in populations that can be arranged as a progressive continuum on the basis of phenotypic, functional and transcriptional attributes. T lymphocytes transition through progressive stages of differentiation that are characterized by a stepwise loss of functional and therapeutic potential in the order from naïve T (T_{N}) cells to T memory stem cells (T_{SCM}) (the most immature antigen experienced T cells), to T central memory (T_{CM}) cells, which patrol central lymphoid organs, to T effector memory (T_{EM}) cells, which patrol peripheral tissues. In contrast to T_{N} cells, memory T cells are capable of rapidly releasing cytokines on restimulation. TCM cells more efficiently secrete IL-2 and T_{EM} have an increased capacity for IFNγ release and cytotoxicity. All antigen-experienced T cells upregulate the common IL-2 and IL-150 receptor (IL-2RO) conferring the ability to undergo homeostatic proliferation in response to IL-15, and also display high amounts of CD95 (also known as FAS), a receptor that provides either costimulatory or pro-apoptotic signals depending on the efficiency of CD95 signaling complex formation and on which particular intracellular signaling proteins are part of the complex. [Gattinoni, L. et al. Nature Revs. Cancer (2012) 12: 671-684].

The term “T cell antigen” as used herein is meant to refer to a protein, lipid (for CD1) or fragment thereof which can be processed into a peptide that can bind to either Class I MHC, Class II MHC, non-classical MHC, or CD1 family molecules (collectively antigen presenting molecules), and in this combination can engage a T cell receptor on a T cell.

The term “T cell epitope” as used herein is meant to refer to a short peptide molecule that binds to a class I or II MHC molecule and that is subsequently recognized by a T cell. T cell epitopes that bind to class I MHC molecules are typically 8-14 amino acids in length, and most typically 9 amino acids in length. T cell epitopes that bind to class II MHC molecules are typically 12-20 amino acids in length. In the case of epitopes that bind to class II MHC molecules, the same T cell epitope may share a common core segment, but differ in the length of the carboxy- and amino-terminal flanking sequences due to the fact that ends of the peptide molecule are not buried in the structure of the class II MHC molecule peptide-binding cleft as they are in the class I MHC molecule peptide-binding cleft.

The term “T cell exhaustion” as used herein refers to a state of T cell hyporesponsiveness with reduced cytotoxic activity, decreased cytokine production, and high expression of inhibitory receptors under persistent Ag and chronic TCR stimulation. It is defined by poor effector function, sustained expression of inhibitory receptors and a transcriptional state distinct from that of functional effector or memory T cells. Exhausted T cells become hyporesponsive via a progressive loss of functionality that is mainly mediated by upregulation of multiple inhibitory receptors, including the inhibitory pathways mediated by PD1 in response to binding of PD1 ligand 1 (PDL1) and/or PDL2. [Wherry E J and Kurachi, M. Nature (2015) 15: 486-99, citing Okazaki T., et al., Nature Immunol. (2013) 14:1212-1218, Odorizzi P M, Wherry E J. J. Immunol. (2012) 188:2957-2965, Araki K., et al. Cold Spring Harb. Symp. Quant. Biol. (2013) 78:239-247]. Exhausted T cells can co-express PD1 together with lymphocyte activation gene 3 protein (LAG3), 2B4 (also known as CD244), CD160, T cell immunoglobulin domain and mucin domain-containing protein 3 (TIM3; also known as HAVCR2), CTLA4 and many other inhibitory receptors [Id., citing Blackburn S D., et al. Nat. Immunol. (2009) 10:29-37]. Typically, the higher the number of inhibitory receptors co-expressed by exhausted T cells, the more severe the exhaustion. While long thought to be a dysfunctional state, T cell dysfunction is now appreciated as a protective mechanism to avoid damage to normal tissues rather than acomplete loss of function. [Wen, S. et al. J. Leuk. Biol. (2021) 110: 585-90, citing Blank, C U et al. Nat. Rev. Immunol. (2019) 19:665-74]. The population of exhausted T cells is highly heterogeneous, and includes such subpopulations as precursor exhausted T cells (T_{PEX} cells), which are defined above. Terminally exhausted T cells preferentially express markers such as CD39, CD244, and TIM3 [Jiang, W. et al. Front. Immunol. (2021) 11: 622509].

The term “T cell factor 1” or (TCF1) as used herein refers to the key transcription factor of the canonical Wnt signaling pathway encoded by Tcf7. The Wnt signaling pathway is evolutionarily conserved and regulates a variety of fundamental processes such as development, cell-fate specification, and maintenance of tissue homeostasis. In mature T cells, TCF1 is known to be critical for the generation of the CD8+ T cell memory response. [Escobar, G. et al. Sci Immunol. (2020) 5 (53): eabb9726, citing Raghu, D. et al. Trends Immunol. (2019) 40: 1149-62].

TCF1 plays stage-specific roles in T cell development. During T cell development, lymphoid progenitors from bone marrow migrate into thymus. Early thymocytes are CD4−CD8− (double-negative (DN)) cells, which can be subdivided into 4 sequential stages of differentiation based on the expression of CD44 and CD25 (DN1, CD44+CD25−; DN2, CD44+CD25+; DN3, CD44−CD25+ and DN4, CD44−CD25−). Cells at the DN3 stage express pre-TCR and transition into DN4 cells after the rearrangement of TCRβ and DN4 cells differentiate through an intermediate CD4-8+, immature (TCR_{low}) single positive (ISP) stage to CD4+CD8+ double-positive (DP) cells, followed by positive and negative selection and ultimately mature into CD4+ or CD8+ single positive (SP) T cells. [Wen, S., et al. J. Leukocyte Biology (2021) 110: 585-90, citing Yui, MA and Rothenberg, EV. Nat. Rev. Immunol. (2014) 14: 529-45]. In the DN1 stage, TCF1 enhances chromatin accessibility and expression of Zap70 and 117r to promote T cells entry the next developmental stage. In the DN2-DN3 stage, TCF1 suppresses the expression of Lef1, Id2, Dtx1, and HesI to prevent T cells from withdrawing from the normal developmental progress transition to malignancy. [Id., citing Wang, F., et al. Cell Mol. Immunol. (2021) 18: 644-59]. In the DP stage, TCF1 cooperates with HeLa E-box binding protein (HEB) to establish the epigenetic and transcription profiles of DP cells through enhancing the chromatin accessibility and expression of the genes involved in multiple pathways, including T cell differentiation, TCR pathway, etc. [Id., citing Emmanuel, A O., et al. Nat. Immunol. (2018) 19: 1366-78]. Moreover, TCF1 has intrinsic histone deacetylase (HDAC) activity that contributes to preserving CD8+ T cell identity by repressing lineage-inappropriate genes (e.g., Cd4, Foxp3, Gata3, and Rorc). [Id., citing Xing, S., et al. Nat. Immunol. (2016) 17: 695-703] In cells committed to the CD8⁺SP T cells, TCF1 interacts with Runx3 to silence Cd4 that ensure their distinct identity and functional divergence. [Id., citing Steinke, F C., et al. Nat. Immunol. (2014) 15: 646-56].

TCF1 modulates formation and function of memory CD8⁺ T cells. TCF1 also has been identified as a key modulator for the formation and function of memory CD8+ T cells. For example, Zhou et al. demonstrated that in memory CD8⁺ T cells, TCF1 induced the optimal expression of Eomes [Eomesodermin, a T-box transcription factor with high homology to T-bet that is expressed by activated CD8+ T cells as well as in resting and activated NK cells; see Shimizu, K. et al. Communication Biol. (2019) 2: article 150 (2019)], through binding to its regulatory gene, which in turn positively regulated IL-2Rβ expression and IL-15 responsiveness that are known to be critical for induction and function of memory T cells. [Zhou, X., et al. Immunity (2010) 33: 229-40]. More recently, Pais-Ferreira and colleagues found that CD8+T_{CM} were almost exclusively derived from effector-phase Tcf7^{high} CD8+ T cells; this finding challenged the previous notion that T_{CM} emerge after pathogen clearance due to the de-differentiation of cytotoxic effector T cells. They identified and verified the TCF1-dependent genes which ensure the stemness of effector-stage Tcf7^{high} CD8+ T cells, including Armxc2, Elovl6, Klf4, Plxdc2, etc. [Id., citing Pais-Ferreira, D., et al. Immunity (2020) 53 (5): 985-1000].

The term “T cell receptor” (TCR) as used herein refers to a complex of integral membrane proteins that participate in the activation of T cells in response to an antigen. The TCR expressed by the majority of T cells consisting of a heterodimer of α and β chains. A small group of T cells express receptors made of γ and δ chains. Among the α/β T cells are two sublineages: those that express the coreceptor molecule CD4 (CD4+ cells), and those that express CD8 (CD8+ cells). These cells differ in how they recognize antigen and in their effector and regulatory functions. The TCR is composed of four distinct signal transducing subunits (CD3-gamma (γ), -delta (δ), -epsilon-(ε), and zeta (ζ) that share a common functional sequence, the immunoreceptor tyrosine-based activation motif (ITAM) [Shores, E W., et al. J. Exp. Med. (1997) 185 (5): 893-900, citing Robey, E. and Fowlkes, BJ. Annu. Rev. Immunol. (1994) 12: 675-705] within their intracytoplasmic domains [Id., citing Reth, M. Nature (Lond) (1989): 338: 383-4; Samelson, L E and Klausner, RD. J Biol. Chem. (1992) 267: 24913-6]. After TCR engagement, phosphorylation of ITAMs leads to the recruitment of SH2 domain-containing proteins (e.g., tyrosine kinases) to the TCR complex and initiation of the T cell activation cascade [Id., citing Samelson, L E and Klausner, RD. J Biol. Chem. (1992) 267: 24913-6; Weiss, A., and Littman, DR. Cell (1994) 76: 263-74; Irving, B A and Weiss, A. Cell (1991) 64: 891-901; Romeo, C., et al. Cell (1992) 68: 889-97]. The CD3 subunits each contain a single ITAM, whereas (contains three ITAMs within its longer cytoplasmic tail. ITAM sequences are conserved but nonidentical. Irving, et al constructed a chimeric protein linking the extracellular and transmembrane domains of CD8 to the cytoplasmic domain of the zeta chain and demonstrated that the CD8/zeta chimera was expressed independently of the TCR and was capable of transducing signals that, by criteria of early and late activation, were indistinguishable from those generated by the intact TCR. [Irving, B A and Weiss, A. Cell (1991) 64: 891-901]. Their data showed that CD8/zeta can activate the appropriate signal transduction pathways in the absence of CD3 gamma, delta, and epsilon, and suggested that the role of CD3 zeta is to couple the TCR to intracellular signal transduction mechanisms.

The term “targeted therapy” as used herein refers to a cancer treatment that, using the genomic and molecular analysis of an individual patient's cancer, is designed to selectively interfere with the product(s) of aberrantly functioning genes that cause cancer and to provide clinical benefit without harming normal cells

The term “TATA box” as used herein refers to a consensus sequence in the promoter region of many eukaryotic genes that binds a general transcription factor and hence specifies the position at which transcription is initiated.

The term “Tbet” as used herein refers to a T_{H}1 cell transcription factor. Differential expression of the T_{H}1 cell transcription factor T bet and a closely related T-box family transcription factor particularly in CD8+ T cells, Eomesodermin (Eomes) facilitate the cooperative maintenance of the pool of antiviral CD8+ T cells during chronic viral infection. [Paley, MA., et al., Science (2012) 338: 1220-125]. During chronic infections, T-bet is reduced in virus-specific CD8+ T cells; this reduction correlates with T cell exhaustion. In contrast, Eomes mRNA expression is up-regulated in exhausted CD8⁺ T cells during chronic infection. [Id.]

The term “T follicular helper (THF) cells” as used herein refers to a distinct subset of CD4+T lymphocytes, specialized in B cell help and in regulation of antibody responses. They develop within secondary lymphoid organs (SLO) and can be identified based on their unique surface phenotype, cytokine secretion profile, and signature transcription factor. They support B cells to produce high-affinity antibodies toward antigens in order to develop a robust humoral immune response and are crucial for the generation of B cell memory. They are essential for infectious disease control and optimal antibody responses after vaccination. Stringent control of their production and function is critically important, both for the induction of an optimal humoral response against thymus-dependent antigens but also for the prevention of self-reactivity. [Gensous, N., et al. Front. Immunol. (2018) doi.org/10.3389/fimmu.2018.01637).

The term “helper T cells” or “T_{H}” cells as used herein refers to effector CD4 T cells that stimulate or “help” B cells to make antibody in response to antigenic challenge. T_{H}2, T_{H}1 and the THF subsets of effector CD4 T cells can perform this function.

The term “TH1 cells” as used herein refers to a lineage of CD4⁺ effector T cells that promotes cell-mediated immune responses and is required for host defense against intracellular viral and bacterial pathogens. They are mainly involved in activating macrophages but can also help stimulate B cells to produce antibody. T_{H}1 cells secrete IFN-gamma, IL-2, IL-10, and TNF-alpha/beta. IL-12 and IFN-γ make naïve CD4+ T cells highly express T-bet and STAT4 and differentiate to T_{H}1 cells. [Zhang, Y., et al. Adv. Exp. Med. Bio. (2014) 841: 15-44].

The term “TH2 cells” as used herein refers to a lineage of CD4+ effector T cells that secrete IL-4, IL-5, IL-9, IL-13, and IL-17E/IL-25. These cells are required for humoral or antibody-mediated immunity and play an important role in coordinating the immune response to large extracellular pathogens. IL-4 makes naïve CD4+ T cells highly express STAT6 and GATA3 and differentiate to TH2 cells. (Zhang, Y., et al. Adv. Exp. Med. Bio. (2014) 841: 15-44).

The term “TH17 cells” as used herein refers to a CD4⁺ T-cell subset characterized by production of interleukin-17 (IL-17). IL-17 is a highly inflammatory cytokine with robust effects on stromal cells in many tissues, resulting in production of inflammatory cytokines and recruitment of leukocytes, especially neutrophils, thus creating a link between innate and adaptive immunity. [Tesmer, LA., et al., Immunol. Rev. (2008) 223: 87-113]. The key transcription factor in TH17 cell development is RORγt.

The term “Treg” or “regulatory T cells” as used herein refers to effector CD4 T cells that inhibit T cell responses and are involved in controlling immune reactions and preventing autoimmunity. The natural regulatory T cell lineage that is produced in the thymus is one subset. The induced regulatory T cells that differentiate from naïve CD4 T cells in the periphery in certain cytokine environments is another subset. Tregs are most commonly identified as CD3⁺CD4⁺CD25⁺FoxP3⁺ cells in both mice and humans. Additional cell surface markers include CD39, 5′ Nucleotidase/CD73, CTLA-4, GITR, LAG-3, LRRC32, and Neuropilin-1. Tregs can also be identified based on the secretion of immunosuppressive cytokines including TGF-beta, IL-10, and IL-35. Cell surface molecules CTLA-4, LAG-3, and neuropilin-1 (Nrp1) impair dendritic cell (DC)-mediated conventional T cell activation: CTLA-4 and LAG-3 outcompete CD28 and T cell receptor expressed on conventional T cells for binding to CD80/86 and MHC class II on DCs, and Nrp1 stabilizes DC-Treg contact, thereby preventing antigen presentation to conventional T cells [Ikebuchi, R., et al. Front. Immunol. (2019) doi.org/10.3389/fimmu.2019.01098].

The term “therapeutic effect” as used herein is meant to refer to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

The term “TIGIT” as used herein refers to a member of the Ig super family and an immune inhibitory receptor.

The term “TIM-3” as used herein refers to a transmembrane protein and immune checkpoint receptor. It is associated with tumor-mediated immune suppression.

The term “tolerance” as used herein refers to a failure to respond to a particular antigen. Tolerance mechanisms that operate in the thymus before the maturation and circulation of T cells are referred to as “central tolerance.” Not all antigens of which T cells need to be tolerant are expressed in the thymus, and therefore central tolerance mechanisms alone are insufficient. Additional tolerance mechanisms exist to restrain the numbers and or function of T cells that are reactive to developmental or food antigens, which are not typically expressed. Tolerance acquired by mature circulating T cells in the peripheral tissues is called “peripheral tolerance.”

The term “toll-like receptor (TLR)” as used herein refers to innate receptors on macrophages, dendritic cells, and some other cells, that recognize pathogens and their products, such as bacterial lipopolysaccharide (LPS). Recognition stimulates the receptor-bearing cells to produce cytokines that help initiate immune responses. For example, TLR-1 is a cell surface toll-like receptor that acts in a heterodimer with TLR-2 to recognize lipoteichoic acid and bacterial lipoproteins. TLR-2 is a cell surface toll-like receptor that acts in a heterodimer with either TLR-1 or TLR-6 to recognize lipoteichoic acid and bacterial lipoproteins. TLR-4 is a cell surface toll-like receptor that, in conjunction with accessory proteins MD-2 and CD14, recognizes bacterial lipopolysaccharide and lipoteichoic acid. TLR5 is a cell surface toll-like receptor that recognizes the flagellin protein of bacterial flagella. TLR 6 is a cell surface toll-like receptor that acts in a heterodimer with TLR2 to recognize lipoteichoic acid and bacterial lipoproteins. TLR3 is an endosomal toll-like receptor that recognizes double-stranded viral RNA. TLR-7 is an endosomal toll-like receptor that recognizes single-stranded viral RNA. TLR-8 is an endosomal toll-like receptor that recognizes single-stranded viral RNA. TLR-9 is an endosomal toll-like receptor that recognizes DNA containing unmethylated CpG.

The terms “TOX” or “thymocytes selection-associated HMG BOX” as used herein refers to a member of a family of transcriptional factors that contain the highly conserved high mobility group box (HMG-box) region. Increasing studies have shown that TOX is involved in maintaining tumors and promoting T cell exhaustion. [Liang, C., et al. Biomark Res. (2021) 9: 20].

The terms “transcription” or “DNA transcription” are used interchangeably herein to refer to copying of one strand of DNA into a complementary RNA sequence by the enzyme RNA polymerase.

The term “transcriptional control” as used herein refers to control of gene expression by controlling when and how often a gene is transcribed.

The term “transcription factor” as used herein refers to a protein required to initiate or regulate transcription in eukaryotes. This includes both gene regulatory proteins as well as the general transcription factors.

The term “transduction” as used herein refers to a process whereby foreign DNA is introduced into another cell via a viral vector.

The term “transfection” as used herein refers to the process of introducing a foreign DNA molecule into a eukaryotic cell by nonviral methods.

The term “transforming growth factor-beta” or “TGF-β” as used herein refers to an inhibitory extracellular signal protein that inhibits the proliferation of several cell types, either by blocking cell-cycle progression in G1 or by stimulating apoptosis. TGF-β binds to cell-surface receptors and initiates an intracellular signaling pathway that leads to changes in the activities of gene regulatory proteins called Smads. This results in complex changes in the transcription of genes encoding regulators of cell division and cell death.

The terms “translation” and “RNA translation” are used interchangeably herein to refer to the process by which the sequence of nucleotides in a messenger RNA molecule directs the incorporation of amino acids into protein. Translation occurs on a ribosome.

The term “translational control” as used herein refers to control of gene expression by selection of which mRNAs in the cytoplasm are translated by ribosomes.

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

The term “tumor associated antigen” or “TAA” refers to a protein or other molecule that has elevated levels on tumor cells but that is also expressed at lower levels on healthy cells.

The term “tumor associated macrophages” or “TAMs” as used herein refers to an immunosuppressive macrophage subtype found in the tumor microenvironment that is involved in the progression and metastasis of cancer. TAMs are broadly considered M2-like, which can be further classified into the M2a phenotype (induced by IL-4 or IL-13), M2b phenotype (IL-10 high, IL-12 low) and M2c phenotype (TNF-α low) according to distinct signal stimuli. They produce abundant growth factors, extracellular matrix (ECM) remodeling molecules and cytokines for the regulation of cancer proliferation via noncoding RNAs, exosomes and epigenetics [Yan, S. and Wan, G. The FEBS Journal (2021) 288 (21): 6174-86. citing Qian, B Z and Pollard, J W. (Cell (2010) 141: 39-51]. Activated M2 macrophages distinctively express arginase 1 (ARG1). TAMs can demonstrate direct inhibition on the cytotoxicity of T-lymphocytes through multiple mechanisms and characteristics of tumor evolution, including immune checkpoint engagement via expression, production of inhibitory cytokines [such as IL-10 and transforming growth factor (TGF)-β] and metabolic activities consisting of depletion of 1-arginine (or other metabolites) and the production of reactive oxygen species (ROS). The suppressive immune response renders cancer cells capable of escaping from immune surveillance.

The term “tumor infiltrating lymphocytes” or “TILs” as used herein refers to a heterogeneous lymphocyte population mainly composed of T lymphocytes that may consist of numerous antitumor effector and/or regulatory T cells (Tregs) hat have invaded a tumor tissue; TILs are key players in the host's immune response to a tumor. [Wang, J., et al. BMC Cancer (2020) 20: 731].

The term “tumor microenvironment” or “TME” as used herein refers to the dynamic and complex ecosystem in which tumor cells exist.

The term “TME macrophages” as used herein refers to macrophages that arise primarily from bone marrow-derived monocytes that are recruited by tumor or stroma-derived chemokines such as colony-stimulating factor 1 (CSF1; also known as M-CSF) and CCL2, to the tumor microenvironment. M1 and M2 phenotypes are differentiated in response to different signal stimuli and are polarized according to the TME, exhibiting strong plasticity, such that macrophages adopt context-dependent phenotypes when stimulated [Yan, S. and Wan, G. The FEBS Journal (2021) 288 (21): 6174-86, citing Murry, P J and Wynn, TA. Nat. Rev. Immunol. (2011) 11: 723-37]. Antitumorigenic M1 macrophages express high levels of tumor necrosis factor (TNF), inducible nitric oxide synthase (iNOS; also known as NOS2) and major histocompatibility complex (MHC) class II molecules, whereas pro-tumorigenic M2 macrophages are marked with high levels of arginase 1 (ARG1), interleukin (IL)-10, CD163, CD204 or CD206 expression. The activation of primary macrophages into M1 or M2 phenotype is mainly induced by interferon-regulatory factor/signal transducer and activator of transcription (STAT) signaling pathways [Id., citing Waqas, S F H., et al. in Nuclear Receptors: Methods and Experimental Protocols, MZ Badr. Ed., Springer, New York, NY, pp. 211-24].

The term “tumor specific antigens” or (“TSA”) refers to a protein or other molecule found on cancer cells only.

The term “Uniform Manifold Approximation and Projection” or UMAP” is an algorithm for dimension reduction based on manifold learning techniques and ideas from topological data analysis. It takes a high-dimensional dataset and reduces it to a low-dimensional plot. A UMAP plot captures local relationships within a cluster as well as global relationships between distinct clusters.

The term “variable (V) domain” as used herein refers to the structural unit of an immunoglobulin or TCR chain that is encoded by the corresponding variable (V) exon.

The term “variable (V) region” as used herein refers to the highly variable N-terminal portion of an Ig or TCR molecule composed of the variable domain that contain the antigen binding site.

The term “volcano plot” as used herein refers to a type of scatterplot that shows statistical significance (P value) versus magnitude of change (fold change). It enables quick visual identification of genes with large fold changes that are also statistically significant. These may be the most biologically significant genes.

The term “wild-type” as used herein refers to the typical form of an organism, strain, gene, protein, nucleic acid, or characteristic as it occurs in nature. Wild-type refers to the most common phenotype in the natural population. The terms “wild-type” and “naturally occurring” are used interchangeably.

The term “zeta-chain-associated protein kinase 70” or “ZAP-70” as used herein refers to a non-src family protein kinase that associates with phosphorylated CD3 zeta chain and plays an important role in TCR-CD3 complex signaling. The main substrate of ZAP70 is the transmembrane adaptor LAT.

### Embodiments

According to one aspect, the present disclosure provides an immunotherapy method for treating a receipient subject with a hematologic cancer comprising administering to the recipient subject an activated and expanded population of genetically engineered CD3+ T cells derived from a healthy donor, wherein the genetic engineering of the donor CD3⁺ T cells in vitro reduces expression of Id3, an inhibitor of DNA binding E protein transcription factors, by at least 25% compared to a control; and wherein the method poses a decreased risk of a graft versus host reaction while preserving graft versus tumor immunity in the recipient subject.

According to some embodiments, the subject is a mammal. According to some embodiments, the subject is a human.

According to some embodiments, the cancer is a hematologic cancer. Hematopoietic cancers are clonal in nature and arise when a target cell sustains multiple genetic alterations to DNA repair genes, oncogenes and/or tumor suppressor genes; many appear to be driven by cancer stem cells (CSCs). [Mak, T W et al. Ch. 20 Hematopoietic cancers, in Primer to the Immune response (2014) Elsevier, Inc., pp. 553 et seq]

According to some embodiments, the cancer is a hematologic cancer. According to some embodiments, the hematologic cancer is a leukemia, a myelodysplastic neoplasm, a myeloma or a lymphoma.

According to some embodiments, the hematologic cancer is a leukemia. Leukemias are tumors that arise from the transformation of a hematopoietic cell in the blood or a hematopoietic precursor in bone marrow; in the latter case, the cancerous progeny of the transformed cell usually make their way into the blood. Leukemias most often occur as “liquid tumors” that are manifested as greatly increased numbers of myeloid, lymphoid, or (more rarely) erythroid lineage cells in the blood and bone marrow.

Leukemia consists of a heterogeneous group of hematological malignancies affecting the cells of all hematopoietic lineages [Sak, K. & Evaraus, H., Curr. Genomics (2017) 18 (1): 3-26], citing Mahbub, A. A. et al. Anticancer Agents med. Chem. (2013) 13 (10): 1601-13; Qin, Y. et al. Eur. J. Pharm. Sci. (2012) 45 (5): 648-56; Kikuchi, H. et al. Intl J. Oncol. (2013) 43(6): 1976-84].

Examples of leukemias include, without limitation, acute lymphoblastic leukemia (also called acute lymphocytic leukemia and ALL), acute myelogenous leukemia (also called acute myeloblastic leukemia, acute myeloid leukemia, acute nonlymphocytic leukemia, AML, and ANLL), acute myeloid leukemia with myelodysplasia, acute promyelocytic leukemia (also called APL), chronic eosinophilic leukemia; chronic granulocytic leukemia (also called chronic myelogenous leukemia, chronic myeloid leukemia, and CML), erythroleukemia; mast cell leukemia; and hairy cell leukemia.

The current treatments for leukemia include chemotherapy, radiation, and bone marrow transplantation, whereas chemotherapy has still remained the most important intervening strategy in treating different types of hematological malignancies [Sak, K. & Evaraus, H., Curr. Genomics (2017) 18 (1): 3-26, citing Zhang, D. et al. Oxid. Med. Cell Longev. (2012) 209843; Liu, X. et al. Nutr. Cancer (2015) 67 (2): 238-249; Mahbub, A A et al. Anticencer. Agents Med. Chem. (2013) 13 (10: 1601-13; Davis, A S, et al. Am. Fam. Physician (2014) 89 (9): 731-38; Lu, H F, et al. Anticancer Res. (2007) 17 (1A): 117-25; Li, R F et al. Exp. Ther. Med. (2015) 9(3): 697-706; Lin, C C, et al. In Vivo (2012) 26 (4): 665-70; Davenport, A. et al. Int. J. Mol. Med. (2010) 25 (3): 465-70; Lee, C Y et al. Oncol. Rep. (2012) 28 (5): 1883-88]. However, standard chemotherapy agents are usually expensive and often associated with toxicity towards normal cells resulting in serious side effects and limiting the overall efficacy of drugs [Id., citing Zu, Y. et al., Planta Med. (2009) 75 (10): 1134-40, Liu, X. et al. Nutr. Cancer (2015) 67 (2): 238-49, Mahbub, A A et al. Anticancer. Agents Med. Chem. (2013) 13 (10): 1601-13, Davis, A S et al. Am. Fam. Physician (2014) 89 (9): 731-8, Li, R F et al. Exp. Ther. Med. (2015) 9(3): 697-706; Davenport, A. et al. Int. J. Mol. Med. (2010) 25(30): 465-70; Li, D. et al. Free Radic. Biol. Med. (2009) 46 (6): 731-36; Lugli, E. et al, Leuk Res. (2009) 33 (1): 140-50; Lee, R. et al. Toxicol. (2004) 195 (2-3): 87-95. In addition, drug resistance also represents a major problem in the current treatment of leukemia [Id., citing Zheng, J. et al. Asian Pac. J. Cancer Prev. (2012) 13 (4): 1119-24, Liu, X. et al. Nutr. Cancer (2015) 67 (2): 238-49, De Martino, L. et al. Mini Rev. Med. Chem. (2011) 11 (6): 492-502, Davenport, A. et al. Int. J. Mol. Med. (2010) 25(30): 465-70, Kilani-Jaziri, S. et al. Chem. Biol. Interact. (2009) 181 (1): 85-94; Chen, F Y et al. Mol. Med. Rep. (2015) 11 (1): 341-48; Shen, J. et al. Int. J. Hyperthermia (2008) 24 (2): 151-9; Cheng, J. et al. Int. J. Nanomedicine (2012) 7: 2843-52]. Such chemoresistance can be either intrinsic or acquired after initial therapy, thus being a main reason for treatment failure [Id., citing De Martino, L. et al. Mini Rev. Med. Chem. (2011) 11 (6): 492-502].

Introduction of targeted therapies has brought about considerable improvements in survival of patients with certain types of leukemia; all-trans retinoic acid (ATRA) for AML and Imatinib against CML represent two examples of the success of target-based therapies [Id., citing Liu, X. et al. Nutr. Cancer (2015) 67 (2): 238-49; Winter, E. et al. Toxicol. In Vitro (2014) 28 (5): 769-77; Noori-Daloii, M R et al. Leuk Res. (2014) 38 (5): 575-80; Kimura, S. et al. Int. J. Oncol. (2014) 19 (1): 3-9; Park, J. et al. Ther. Adv. Hematol. (2011) 2(5): 335-52]. Agents that force immature leukemia cells to undergo terminal differentiation (“differentiation therapy”) may be a less toxic alternative to treat hematopoietic neoplasms [Id., citing De Martino, L. et al. Mini Rev. Med. Chem. (2011) 11 (6): 492-502, Chen, H. et al. Molecules (2012) 17 (11): 13424-38; Isoda, H. et al. Chem. Biol. Interact. (2014) 220: 269-77; Tsolmon, S. et al. Mol. Nutr. Food Res. (2011) 55 (Suppl. 1): S93-S102; Chen, Y. et al. Blood (2013) 121 (18): 3682-91; Lee, C Y et al. Oncol. Rep. (2012) 28 (5): 1883-88, Li, D. et al. Free Radic. Biol. Med. (2009) 46 (6): 731-6, Chen, H. et al. Cell Biol. Int. (2013) 37 (11): 1215-1224; Wang, M. et al. Arch. Pharm Res. (2012) 35 (1): 129-135; Yang, H. et al. Oncotarget (2014) 5(18): 8188-8201]. Indeed, granulocytic differentiation induced by ATRA plus arsenic trioxide (ATO) has proven to be a highly curative standard of care for treatment of acute promyelocytic leukemia (APL), an AML subtype. [Kantarjian, H M et al. Cancer (2018) 124: 4301-13]. However, application of differentiation therapy can be effective only in certain forms of leukemia and the treatment might be accompanied by severe side effects as well as development of resistance [Sak, K. & Evaraus, H., Curr. Genomics (2017) 18 (1): 3-26, citing Qin, Y. et al., Eur. J. Pharm. Sci. (2012) 45 (5): 648-56; Zhang, K. et al. Cancer Sci. (20008) 99 (4): 689-95; Hui, H. et al. Gene (2014) 551 (20: 230-35; Nakazato, T. et al. Haematologica (2005) 990 (3): 317-25; Philchenkov, A A et al. Ukr. Biokhim. Zh. (2010) 82 (2): 104-110; Nakazaki, E. et al. Eur. J. Nutr. (2013) 52 (1): 25-35].

Management strategies of CML have made a significant progress after the discovery of Imatinib as a selective protein tyrosine kinase inhibitor against BCR-ABL, which is formed as a consequence of the reciprocal translocation between chromosomes 9 and 22; its constitutive tyrosine kinase activity contributes to antiapoptotic mechanisms, uncontrolled cell proliferation and survival advantage in CML [Id., citing Zhu, J F et al. PLoS One (2011) 6 (8): e23720; Kim, J H et al., Leuk Res. (2012) 36 (9): 1157-64; Noori-Daloii, M R et al. Leuk Res. (2014) 38 (5): 575-80; Yang, H. et al. Oncotarget (2014) 5 (18): 8188-8201; Iwasaki, R. et al. Cancer Sci. (2009) 100 (2): 349-56; Tolomeo, M. et al. Cancer let. (2008) 265 (2): 289-97; Solmaz, S. et al. Nutr. Cancer (2014) 66 (4): 599-612; Lust, S. et al. Mol. Nutr. Food Res. (2010) 54 (6): 823-32; Monteghirfo, S. et al. Mol Cancer Ther. (2008) 7 (9): 2692-2702; Valdes, A. et al. Electrophoresis (2012) 33 (15): 2314-27]. However, despite the initial therapeutic efficiency of Imatinib, development of resistance and disease relapse are still serious problems for most patients [Tolomeo, M. et al. Cancer let. (2008) 265 (2): 289-97; Solmaz, S. et al. Nutr. Cancer (2014) 66 (4): 599-612; Lust, S. et al. Mol. Nutr. Food Res. (2010) 54 (6): 823-32; Monteghirfo, S. et al. Mol Cancer Ther. (2008) 7 (9): 2692-2702]. In addition, the use of new generation inhibitors specifically targeting the tyrosine kinase domain of Bcr-Abl, such as Dasatinib and Nilotinib, can also be limited due to emergence of resistance and adverse effects of these agents [Kim, J H et al., Leuk Res. (2012) 36 (9): 1157-64, Iwasaki, R. et al. Cancer Sci. (2009) 100 (2): 349-56, Tolomeo, M. et al. Cancer let. (2008) 265 (2): 289-97; Solmaz, S. et al. Nutr. Cancer (2014) 66 (4): 599-612].

Emerging therapies in AML include FLT3 inhibitors, IDH1/IDH2 inhibitors, gemtuzumab oxogamicin (GO) and novel antibodies targeting CD33 and CD123, venetoclax as a BCL3 inhibitor and the introduction of checkpoint inhibitors. [Antarjian, H M et al. Cancer (2018) 124: 4301-13].

According to some embodiments, the subject with a leukemia is a child. According to some embodiments, the subject with a leukemia is an adult. According to some embodiments, the leukemia is an acute lymphocytic leukemia. According to some embodiments, the leukemia is a chronic lymphocytic leukemia. According to some embodiments, the leukemia is an acute myeloid leukemia. According to some embodiments, the leukemia is a chronic myeloid leukemia.

According to some embodiments, the hematologic cancer is a myeloproliferative neoplasm. The abnormal proliferation of one or more terminal myeloid cell lines in the peripheral blood gives rise to a heterogeneous group of disorders called myeloproliferative neoplasms. Chronic myeloid leukemia (CML), polycythemia vera (PV), essential thrombocythemia (ET), and primary myelofibrosis (PMF) are four classic types of myeloproliferative neoplasms. WHO classification also included chronic neutrophilic leukemia (CNL), chronic eosinophilic leukemia (CEL), and MPN, unclassifiable. Out of the classic types of MPNs, CML is BCR-ABL1 positive, but PV, ET, and PMF are BCR-ABL1 negative.

The BCR-ABL1-negative myeloproliferative neoplasms (MPNs) are clonal disorders of the hematopoietic stem cell, mainly characterized by hyperproliferative bone marrow with varying degrees of reticulin/collagen fibrosis, extramedullary hematopoiesis, abnormal peripheral blood count, and constitutional symptoms. [Iurlo, A. et al. Intl J. Mol. Sci. (2019) 20 (8): 1839]. The major causes of morbidity and mortality in these patients are most commonly represented by thrombo-hemorrhagic events and less frequently by infectious complications, and/or transformation to blast phase, often termed secondary acute myeloid leukemia (AML) or blast-phase MPN (MPN-BP).

According to some embodiments, the hematologic cancer is a myeloma. Myelomas are tumors of fully differentiated plasma cells that are present either as solid masses or as dispersed clones in the bone marrow, blood or tissues. Unlike normal plasma cells, which do not divide after they differentiate, myeloma cells continue to proliferate in an uncontrolled way and synthesize large amounts of Ig chains. The term “myeloma” as used herein refers to a plasma cell tumor that secretes large quantities of an Ig protein of usually unknown specificity. When tumors are present in multiple body sites, the disease is referred to as multiple myeloma (MM).

Normal plasma cells cannot divide and so die soon after secreting antigen-specific antibody. In contrast, cancerous plasma cells divide uncontrollably and express huge quantities of antibodies or single Ig chains of unknown antigenic specificity.

The progression of multiple myeloma (MM), a B cell malignancy, begins with the precursor pathogenic state of monoclonal gammopathy of undetermined significance (“MGUS”). In the absence of clinical symptoms, MGUS is diagnosed by quantifying the amount of immunoglobulin present in both the bloodstream and BM, specifically with a plasma cell population of <10% in the BM. The Ig protein produced by the malignant plasma cell is called a paraprotein, IgM paraprotein, para-IgM or M protein. Evidence indicates that MGUS, previously characterized by myeloma cell growth without bone destruction or other organ involvement, is in fact associated with alterations in the bone. Epidemiologic evidence has shown that patients with MGUS suffer from a significantly increased fracture risk, and that the prevalence of MGUS is increased in patients with osteoporosis. [Fairfield, H. et al., Ann. NY Acad. Sci. (2016) 1364 (1): 32-51, citing Drake, MT. J. Bone Miner. Res. (2014) 29: 2529-33] It has been demonstrated that the onset of MGUS is concurrent with the deterioration of both auxiliary and appendicular microarchitecture leading to skeletal fragility [Id., citing Drake, MT. J. Bone Miner. Res. (2014) 29: 2529-33].

The disease state transitions into either smoldering MM (SMM) or MM (if clinically manifested), with a plasma cell content exceeding 10%, [Fairfield, H. et al., Ann. NY Acad. Sci. (2016) 1364 (1): 32-51, citing Berenson, JR e t al. Br. J. Haematol. (2010) 150: 28-38].

Smoldering multiple myeloma (SMM) is an intermediate clinical stage between MGUS and MM. It is classified as having high serum or urinary monoclonal protein as well as clonal BM plasma cells in the range of 10-60%, in the absence of additional myeloma-defining events [Id., citing Glavey, SV & Ghobrial, IM. Expert Rev. Hematol. (2015) 8: 273-5] such as hypercalcaemia, renal insufficiency, anemia, or bone lesions. [Id., citing Rajkumar, S V et al. Lancet Oncol. (2014) 15: e538-3548]. MGUS progresses to MM at a rate of 1-2% of patients per year; this transition is likely influenced by the presence of mutational diversity or clonality of MM cell populations as well as changes in the local bone marrow (BM) and other systemic factors [Id., citing Jemal, A. et al. C A Cancer J. Clin. (2009) 59: 1-25; Pawlyn, C. et al. Blood (2015) 125 (5): 831-40; Barlogie, B. et al. Blood (2004) 103: 20-32; Rollig, C. et al. Lancet (2014) 385]. MM cells are thought to initially create a plasmacytoma, a single tumor, and then develop into multiple lesions to form the disease of multiple myeloma. [Id., citing Lorsbach, R B et al. Am. J. Clin. Pathol. (2011) 136: 168-182]. [00175]

MM begins as monoclonal gammopathy of undetermined significance (MGUS), progresses to smoldering (asymptomatic) myeloma and finally becomes overt (symptomatic) myeloma, resulting in BM infiltration and osteolytic lesions. [Fairfield, H. et al., Ann. NY Acad. Sci. (2016) 1364 (1): 32-51].

According to some embodiments, the hematologic malignancy is a monoclonal gammopathy of undetermined significance (MGUS). According to some embodiments, the hematologic malignancy is a smoldering myeloma. According to some embodiments, the hematologic malignancy is a multiple myeloma.

According to some embodiments, the cancer is a lymphoma. Lymphomas are solid cancers of the lymphatic system that initiate from the malignant transformation of a single lymphocyte. The affected lymphocyte usually is located in a lymph node, but may be resident in another organized lymphoid tissue outside the bone marrow, such as the spleen or thymus. When the transformed lymphocyte is positioned in a diffuse lymphoid tissue, such as the gut associated lymphoid tissue (GALT), the lymphoma that develops is said to be extranodal. Lymphomas almost always depend on surrounding stromal cells for survival and growth factors as well as vital intercellular contacts, and so are generally restricted to sites within tissues. From its initiation site, a lymphoma tends to spread to additional secondary lymphoid tissues and eventually to non-lymphoid organs. Occasionally, a lymphoma cell undergoes additional mutations that allow it to survive and circulate in the blood, i.e., it becomes a leukemic cell. The disease then may be called a “leukemia/lymphoma.” [Mak, T W et al. Ch. 20 Hematopoietic cancers, in Primer to the Immune response (2014) Elsevier, Inc., pp. 573-574].

The progression of any lymphoma can be described in four stages:

In Stage I, one or more diseased lymph nodes are present in a single group of lymph nodes in one particular lymphoid tissue of the body.

In stage 2, diseased lymph nodes are present in more than one group of lymph nodes, but all diseased nodes are contained either above or below the diaphragm. Tumor cells may also be present in a single organ near an affected node.

In stage III, diseased lymph nodes are present in two or more groups on both sides of the diaphragm. Tumor cells also may be present in the spleen and/or another organ near an affected node.

In stage IV. There is wide dissemination of tumor cells into multiple lymph nodes, bone marrow, liver and multiple organs. [Mak, T W et al. Ch. 20 Hematopoietic cancers, in Primer to the Immune response (2014) Elsevier, Inc., pp. 573-574]

Lymphomas display a tumor microenvironment (TME), with huge differences amongst the various forms. Hodgkin lymphoma (HL), both classic HL and nodular lymphocyte predominant HL, as well as several T cell lymphoma entities, such as angioimmunoblastic T-cell lymphomas (AITL), predominantly (>80% of the tumor mass) consist of TME cells. In indolent B-cell lymphomas, such as follicular lymphoma (FL) or marginal zone lymphomas, the TME constitutes about 50% of the cellular mass. In aggressive lymphomas, such as diffuse large B-cell lymphomas (DLBCL), the proportion of the TME varies and is generally lower. In Burkitt lymphoma, plasmablastic lymphoma and lympoblastic T cell and B cell lymphomas, the TME is barely existent. [Menter, T. & Tzankov, A. Pathobiology (2019) 86: 225-36].

### Hodgkin Lymphoma

Hodgkin's lymphoma is a lymphoma in which the tumor mass is made up of a reactive infiltrate of nontransformed lymphocytes, macrophages and fibroblasts plus scattered, malignant Reed-Sternberg cells. Reed-Sternberg cells are large, abnormal lymphocytes that may contain more than one nucleus, are clonal in their growth and are the tumor cells of the malignant mass. While of the B-cell lineage, they lack common B-cell specific surface markers such as CD19 and CD79a as well as Ig gene transcripts. [Hertel, C B et al. Oncogene (2002) 21: 4908-20]. Hodgkin lymphoma most commonly affects lymph nodes in the neck or in the area between the lungs and behind the breastbone. It can also begin in groups of lymph nodes under an arm, in the groin, or in the abdomen or pelvis. If it spreads, it may spread to the lung, spleen liver, bone marrow or bone.

There are two major categories of Hodgkin lymphoma: classical Hodgkin lymphoma, which is divided into 4 subtypes based on the appearance of lymph node structure and cells, and nodular lymphocyte-predominant Hodgkin lymphoma.

Classical Hodgkin lymphoma (cHL) represents about 95% of cases of Hodgkin lymphoma. It is diagnosed when characteristic abnormal lymphocytes, (Reed-Sternberg cells) are found. There are 4 subtypes of cHL.

Nodular sclerosis Hodgkin lymphoma, the most common subtype of cHL, affects up to 80% of people diagnosed with cHL. It is most common in young adults, especially women. In addition to Reed-Sternberg cells, there are bands of connective tissue (called fibrosis) found in the lymph node. This type of lymphoma often affects the lymph nodes in the mediastinum.

Lymphocyte-rich classic Hodgkin lymphoma affects up to 6% of individuals with cHL. It is more common in men and usually affects areas other than the mediastinum.

In addition to Reed-Sternberg cells, the lymph node tissue contains many normal lymphocytes.

Mixed cellularity Hodgkin lymphoma occurs most often in older adults. It sometimes develops in the abdomen and carries many different cell types, including large numbers of Reed-Sternberg cells.

Lymphocyte-depleted Hodgkin lymphoma is the least common subtype of cHL, and represents only about 1% of patients with cHL. It is most common in older adults; people with HIC; and people in non-industrial countries. The lymph node contains almost all Reed-Sternberg cells.

Nodular lymphocyte-predominant Hodgkin lymphoma (also called LPHL, lymphocyte-predominant Hodgkin lymphoma, and NLPHL). is a rare type of Hodgkin lymphoma marked by the presence of lymphocyte-predominant cells, which used to be called popcorn cells. These cells are different from the typical Reed-Sternberg cells found in classic Hodgkin lymphoma. Nodular lymphocyte-predominant Hodgkin lymphoma may change into diffuse large B-cell lymphoma.

### Non-Hodgkin Lymphoma

Non-Hodgkin's lymphoma (NHL) is a heterogeneous group of lymphomas in which the solid tumor mass consists almost entirely of malignant lymphocytes. [Mak, T W et al. Ch. 20 Hematopoietic cancers, in Primer to the Immune response (2014) Elsevier, Inc., pp. 573-574] NHL may be described by how quickly the cancer is growing. An “indolent or low grade lymphoma' is a type of lymphoma that tends to grow and spread slowly, and has few symptoms. When indolent lymphoma is in stages 1 and 2, it is called localized disease. An “aggressive lymphoma” “high-grade lymphoma” or intermediate-grade lymphoma” is a type of lymphoma that grows and spreads quickly and has severe symptoms. In children, aggressive non-Hodgkin lymphoma is more common. Some types of lymphoma cannot be easily classified as indolent or aggressive.

### Subtypes of NHL

There are more than 60 NHL subtypes of NHL that tend to mimic stages of normal B cell differentiation. [Mancuso, S. et al. Immunity & Aging (2018) 15: 22].

About 90% of individuals in western countries with lymphoma have B-cell lymphoma.

T cell lymphomas T cell lymphomas make up approximately 10%-15% of lymphoid malignancies. [Armitage, J. O. Am. J. Hematol. (2017) 92 (7): 706-15, citing 1]. The frequency of these lymphomas varies geographically, with the highest incidence in parts of Asia. [Id., citing Anderson, J R et al. Ann. Oncol. (1998) 9: 717-20]T-cell lymphomas can be divided into those of precursor T-cells (i.e., precursor T-lymphoblastic lymphoma) and those arising in more mature T-cells, with the latter termed peripheral T-cell lymphomas (PTCLs). As is true for B-cell lymphomas, a subset of PTCLs such as mycosis fungoides and the CD30-positive cutaneous lymphoproliferative disorders (i.e., cutaneous anaplastic large-cell lymphoma and lymphomatoid papulosis) have a prolonged natural history. The aggressive PTCLs are associated with a short survival. These can be subdivided into those of primarily nodal origin and those that typically present in specific extranodal sites and are often associated with characteristic clinical syndromes. [Armitage, J. O. Am. J. Hematol. (2017) 92 (7): 706-15]

Less than 1% of individuals with lymphoma have NK-cell lymphoma.

The most common subtypes of T and NK-cell lymphoma are:

Anaplastic large cell lymphoma, primary cutaneous type, which only involves the skin. It is often indolent, although aggressive subtypes are possible.

Anaplastic large cell lymphoma, systemic type is an aggressive form of lymphoma that makes up about 2% of all lymphomas and about 10% of all childhood lymphomas. An increased amount of the ALK-1 protein may be found in the cancer cells, which leads to a better prognosis.

Breast implant-associated anaplastic large cell lymphoma arises in areas near breast implants. It is usually less aggressive than the systemic type of anaplastic large cell lymphoma.

Peripheral T-cell lymphoma, not otherwise specified (NOS) is an aggressive form of lymphoma most common in individuals older than 60 and makes up about 6% of all lymphomas in the United States and Europe. The cells of this lymphoma vary in size, and have CD4 or CD8 on their surface.

Angioimmunoblastic T-cell lymphoma (AITL) is an aggressive form of lymphoma characterized by enlarged often tender lymph nodes, fever, weight loss, rash and high levels of Igs in the blood.

Adult T-cell lymphoma/leukemia (human T cell lymphotropic virus type I positive) is caused by the human T-cell lymphotropic virus type 1. It is an aggressive disease that often involves the bone and skin. Lymphoma cells are often found in the blood.

Extranodal NK/T cell lymphoma nasal type is an aggressive type of lymphoma that is very rare in the United States and Europe in general, but more common in Asian and Hispanic communities. While most often involving the nasal area and sinuses, it also can involve the gastrointestinal tract, skin, testicles or other areas in the body.

Enteropathy-associated T cell lymphoma is rare in the United States but is more common in Europe. It is an aggressive form of T-cell lymphoma that involves the intestines. Some with this subtype have celiac disease or a history of gluten intolerance.

Hepatosplenic T-cell lymphoma is an aggressive form of peripheral T cell lymphoma that involves the liver and spleen. It occurs most often in teenaged and young men.

Subcutaneous panniculitis-like T cell lymphoma is a high risk aggressive lymphoma. It is a form of peripheral T cell lymphoma that is similar to hepatosplenic T cell lymphoma. It involves the tissue under the skin, and is often first diagnosed as panniculitis (inflammation of fatty tissues).

Mycosis fungoides is a rare T cell lymphoma that primarily involves the skin. It often has a very long and indolent course but may become more aggressive and spread to lymph nodes or internal organs.

There are three major types of NHL in children. These include: aggressive mature B cell non-Hodgkin lymphoma; lymphoblastic lymphoma; and anaplastic large cell lymphoma. Aggressive mature B-cell non-Hodgkin lymphomas include Burkitt lymphoma/leukemia; diffuse large B cell lymphoma; and primary mediastinal B-cell lymphoma. Lymphoblastic lymphoma is a type of lymphoma that mainly affects T-cell lymphocytes. Anaplastic large cell lymphoma is a type of lymphoma that mainly affects T-cell lymphocytes. It usually forms in the lymph nodes, skin, or bone, and sometimes forms in the gastrointestinal tract, lung, tissue that covers the lungs, and muscle. Patients with anaplastic large cell lymphoma have a receptor, called CD30, on the surface of their T cells. In many children, anaplastic large cell lymphoma is marked by changes in the ALK gene that makes a protein called anaplastic lymphoma kinase. [www.cancer.gov/types/lymphoma/patient/child-nhl-treatment-pdq#:˜:text=Childhood %20non%2DHodgkin%201ymphoma%20is,of%20childhood%20non%2DHodgkin%20lymphoma.&text=Some%20types%20of%20non%2DHodgkin%201ymphoma%20are%20rare%20in%20children, visited Jul. 22, 2024].

According to some embodiments, the hematologic cancer is a lymphoma. According to some embodiments, the subject with a lymphoma is a child. According to some embodiments, the subject with a lymphoma is an adult. According to some embodiments, the lymphoma is a Hodgkin lymphoma. According to some embodiments, the lymphoma is a non-Hodgkin lymphoma. According to some embodiments, the non-Hodgkin lymphoma is a B cell lymphoma. According to some embodiments, the non-Hodgkin lymphoma is a T cell lymphoma.

Cellular immunotherapy. According to some embodiments, the donor T cells are autologous to the recipient subject. According to some embodiments, the donor T cells are allogeneic to the recipient subject. According to some embodiments, CD3⁺ T cells are purified from mononuclear cells collected from umbilical cord blood or from adult peripheral blood (PB). According to some embodiments, the T cells are derived from adult peripheral blood after mobilization of peripheral blood stem cells with an infusion of G-CSF. According to some embodiments, a population of mononuclear cells comprising a population of T cells is obtained from a healthy donor.

According to some embodiments, CD3+ T cells comprising CD4+ T cells, CD8+ T cells or both are isolated from a population of mononuclear cells by negative selection. According to some embodiments, the CD3+ T cells comprising CD4+ T cells, CD8+ T cells or both isolated from the population of mononuclear cells by negative selection express a nonexhausted phenotype. According to some embodiments, CD25+ Tregs are depleted by positive selection.

According to some embodiments, the Id3 gene of the population of CD3+ T cells comprising CD4+ T cells, CD8+ T cells or both is edited by genetic engineering in vitro to ablate expression of Id3. According to some embodiments, the editing that ablates the gene expression of the Id3 gene is accomplished by CRISPR/Cas9 in vitro. According to some embodiments, the editing reduces expression of the gene encoding Id3 by at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or 100% compared to a control.

According to some embodiments, the genetically engineered population of T cells comprising the edited Id3 gene is expanded and activated in vitro in the presence of one or more cytokines. According to some embodiments, the cytokine is IL-2, IL7, IL15, IL-18, IL-21 or a combination thereof. [See, e.g., Gattinoni, L. et al. Nature Reviews Cancer (2012) 12: 671-84, at 679-80; Cieri, N. et al. Blood (2013) 121 (4): 573-84; Xu, X-J et al. Oncotarget (2016) 7 (50): 82354-68; Xu, Y et al., Blood (2014) 123 (24): 3750-9; Bachmann, M F and Oxenius, A. EMBO Rep. (2007) 8 (12): 1142-48]. Dynabeads CD3/CD28 are exemplary activating agents for mouse and human T cells (ThermoFisher Scientific).

### Exemplary Methods for Assessing Activation and Proliferation

Activation Markers. Activated T cells proliferate and express surface receptors, such as CD25 (the IL-2 receptor) and CD71 (the transferrin receptor), or costimulatory molecules, such as CD26, CD27, CD28, and CD154. Both surface marker expression and cell proliferation can be assessed by flow cytometry. In addition, T-cell surface proteins such as CD30 are released by activated T cells into the circulation as sCD30 and can be measured in serum or plasma by enzyme-linked immunoassays. Alternatively, T-cell activation can be monitored by cytokine production and gene expression assessment (e.g., Elispot).

The term “Enzyme linked immunospot” or ELISpot, as used herein refers to a technique that was developed for the detection of secreted proteins, such as cytokines and growth factors. It is performed using a PVDF or nitrocellulose membrane 96-well plate pre-coated with an antibody specific to the secreted protein. Cells are added to the plate and attach to the coated membrane. Cells are then stimulated and the secreted protein binds to the antibody. Next, a detection antibody is added that binds specifically to the bound protein. The resulting antibody complex can be detected either through enzymatic action to produce a colored substrate or with fluorescent tags. The membrane can be analyzed by manually counting the spots or with an automated reader designed for this purpose. Each secreting cell appears as a spot of color or fluorescence.

Another approach to assess the state of T cell activation is to examine the upregulation of cytokines. CD4+TH1 cells are the primary source for the pro-inflammatory cytokines IFN-γ, IL-2, and TNFβ, whereas IL-4, IL-5, IL-9, IL-10, IL-13 and IL-25 are produced by CD4+TH2 cells. [Id., citing Zhu, J. and Paul, WE. Blood (2008) 112: 1557-69]. Activated CD8+ effector and memory T cells also produce IL-2, IL-10, TNFα, and IFNγ. [Id., citing Zhang, N. and Bevan, MJ. Immunity (2011) 35: 161-8]. Cytokines can be monitored inside activated T cells (e.g., by flow cytometry) or the release of cytokines from stimulated T cells can be followed (e.g., by ELISAs, antibody-coated beads and flow cytometry, ELISPOT).

Since T cell activation is accompanied by upregulation of genes, such as those coding for cell division, cytokines, adhesion molecules or cytotoxic peptides, these genes can be followed individually by qPCR. In addition, gene signatures have been described using DNA microarrays, although compared to qPCR, they are less sensitive to detect small changes in gene expression due to background noise.

Proliferation. Proliferation also can be detected by incorporation of labeled DNA precursors. Alternatively, cell division and growth can be detected by incubating cells with a colorimetric substrate, such as the tetrazolium salt MTT (3-(4,5-dimethylthiazol-2-yl)-2,5-diphenyl-tetrazolium bromide; thazolyl blue), which is reduced by mitochondrial succinate dehydrogenase. The succimidyl ester of carboxy fluorescein diacetate (CFSE), which irreversibly binds to proteins both on the cell surface and intracellularly by reaction with lysine and other amine groups, also can be used to study cell proliferation. During cell division, CFSE labeling is distributed equally between daughter cells, thereby losing fluorescence, which can be followed by flow cytometry.

According to some embodiments, the administering of the genetically engineered CD3+ T cells comprising reduced expression of the Id3 gene is by infusion.

According to some embodiments, the method includes administering a short course of chemotherapy to reduce/kill the subject's population of T cells prior to infusion. This short course of chemotherapy is referred to as “lymphodeletion”, which creates a favorable immune environment for T cell transplantation. According to some embodiments the lymphodepleting therapy comprises fludarabine at 30 mg/m2 per day for 4 days and cyclophosphamide at 500 mg/m2 per day for 2 days (Fabrizio, V A et al. Blood Adv. (2022) 6 (7): 1961-8]. According to some embodiments, reduced levels of chemotherapy and a modified lymphodepleting regimen that also included alemtuzumab (Lemtrada) are administered to more effectively deplete T-cells and ensure CAR T-cells can expand and persist in the body longer. [Jain, N. www.targetedonc.com/view/lymphodepletion-improves-results-for-allogenic-car-t-cell-therapy-in-all, visited Jul. 29, 2024].

According to some embodiments, the expanded activated population of genetically engineered CD3+ T cells comprising CD4+ T cells, CD8+ T cells or both comprising the edited Id3 gene is characterized by an improved ability to secrete effector cytokines; an improved cytotoxicity, or both against tumor cells compared to the population of mononuclear cells and a reduced risk of graft versus host disease.

### CAR-T Cells Containing Extopically Expressed Id3

According to another aspect, the present disclosure provides an immunotherapy method for treating a recipient subject with a hematologic cancer comprising:
(a) genetically engineering a population of CD3+ T cells derived from a healthy donor: (i) to express a chimeric antigen receptor (CAR) that specifically binds to a tumor antigen; and (ii) to ectopically express Id3 (Id30E);
(b) activating and expanding the population of genetically engineered CD3+-CAR IDOE T cells of (a);
(c) administering the activated and expanded population of genetically engineered CD3+CAR-Id^{OE} T cells of (b), wherein the cell population is characterized by:

An enhanced persistence and enhanced ability to expand; and/or
a lower frequency of terminally exhausted cells of phenotype PD-1+TIM3+; and/or
a higher frequency of progenitor exhausted cells (T_{PEX}) cells of phenotype PD-1⁺TIM3⁻; and/or
a higher frequency of central memory phenotype (CD62+CD45RA−) upon ex vivo culture in IL2, IL7 and IL-15; and/or
an enhanced ability to product IL-2 secretion and to proliferate upon antigen rechallenge in ex vivo culture; and/or
augmented memory protection against tumor challenge in a mouse leukemia model; and/or

improved overall survival without tumor; and/or
a combination thereof.

According to some embodiments, the subject is a mammal. According to some embodiments, the subject is a human.

According to some embodiments, the cancer is a hematologic cancer.

According to some embodiments, the hematologic cancer is a leukemia, a myelodysplastic neoplasm, a myeloma or a lymphoma.

According to some embodiments, the donor T cells are allogeneic to the recipient subject. According to some embodiments, CD3⁺ T cells are purified from mononuclear cells collected from umbilical cord blood or from adult peripheral blood (PB). According to some embodiments, the T cells are derived from adult peripheral blood after mobilization of peripheral blood stem cells with an infusion of G-CSF. According to some embodiments, a population of mononuclear cells comprising a population of T cells is obtained from a healthy donor.

According to some embodiments, CD3+ T cells comprising CD4+ T cells, CD8+ T cells or both are isolated from a population of mononuclear cells by negative selection. According to some embodiments, the CD3+ T cells comprising CD4+ T cells, CD8+ T cells or both isolated from the population of mononuclear cells by negative selection express a nonexhausted phenotype. According to some embodiments, CD25+ Tregs are depleted by positive selection.

According to some embodiments, the population of donor T cells comprising CD4+ T cells, CD8+ T cells or both are transduced on RetroNectin (Takara Bio)-coated plates by a retroviral vector comprising a nucleic acid encoding a synthetic chimeric antigen receptor (CAR) that specifically binds a cancer antigen to stably express the cancer antigen-specific CAR.

According to some embodiments, the CAR comprises an extracellular antigen recognition domain, a spacer/hinge region and transmembrane domain; and an intracellular signal transduction domain.

According to some embodiments, the intracellular signal transduction domain comprises a CD3 (T cell activation chain. According to some embodiments, the intracellular signal transduction domain comprising a CD3 (activation chain may comprise one or more co-stimulatory moleules, e.g., CD28. According to some embodiments, the CD28 domain can be followed by a 4-1BB signaling domain, an OX-40 signaling domain or combinations thereof.

CAR T-cells are commercially available to order from a commercial source such as Creative Biolabs (NY USA), which provides custom construction and production services for CARs and also provides premade CAR constructs stock. Custom made CAR T-cells may also be obtained from Promab Biotechnologies (CA USA), which can provide specifically designed CAR T-cells.

### Targeting Antigens

According to some embodiments, for each antigen, the extracellular antigen recognition domain comprises a single-chain variable fragment (scFv) derived from a monoclonal antibody specific for that antigen. According to some embodiments, each CAR is specific for a tumor associated antigen or a tumor specific antigen.

Leukemia-associated antigens (LAA) can be broadly characterized as: (1) unique leukemia-specific antigens or neoantigens, arising as a consequence of mutations or chromosomal translocations (e.g. BCR-ABL in B cell acute lymphoblastic leukemia [Neelakantan, P. and Rezvani, K. Chapter 81 in Handbook of Biologically Active Peptides (2013) Elsevier, Inc. pp. 596-602, citing Bocchia, M., et al. Blood (1996) 87: 3587-92]); (2) aberrantly expressed or overexpressed self-antigens e.g. Wilms tumor (WTI1), a zinc finger transcription factor [Id., citing Gao, L., et al. Blood (2000) 95: 2198-208]; proteinase-3 (PR3) [Id., citing Moldrem, J., et al. Blood (1996) 88: 2450-7; Rezvani, K. et al. Blood (2008) 102: 2892-900]; the receptor for hyaluronic-acid-mediated motility (RHAMM, also known as CD168)[Id., citing Schmitt, M., et al. Blood (2008) 111: 1357-65] and preferentially expressed antigen of melanoma (PRAME) [Id., citing Quintarelli, C., et al. Blood (2008) 112: 1876-85; Rezvani, K. et al. Blood (2009) 113: 2245-55]; (3) Viral antigens e.g. Ebstein-Barr virus (EBV); and 4) Allo antigens, such as the minor histocompatibility antigens (mHAgs), HA-1 and HA-2 [Id., citing Goulmy, E. Curr. Opin. Immunol. (1996) 8: 75-81].

An ideal target antigen for immunotherapy is preferentially expressed on leukemic progenitor cells, is intrinsic to leukemic survival so that viable tumor escape by down regulation of the antigen cannot occur, and induces a strong cytotoxic T-cell (CTL) response.

Acute lymphoblastic leukemias (also called acute lymphocytic leukemia) (ALL) (among adults, B-cell lineage represents 75 percent of cases) and B cell chronic lymphocytic leukemia (CLL) cells express a variety of specific antigens such as CD20, CD22, CD33, and CD52.

For example, CD19 is an IgSF surface glycoprotein of 95 kDa that is expressed from the earliest stages of B cell development until plasma cell terminal differentiation, when its expression is lost.

For example, CD20 (encoded by the MS4A1 gene) is a B cell differentiation antigen expressed at most stages of B cell development, apart from early pro-B cells, plasmablasts, and plasma cells.

For example, CD22 is an inhibitory receptor of the Siglec family that is predominantly expressed on B cells.

For example, CD33 is a sialoadhesin molecule and a member of the immunoglobulin supergene family. It is present in more than 80% of patients with AML but is absent from pluripotent hematopoietic stem cells and is expressed by the immature myelomonocytic population in acute myeloid leukemia (AML).

For example, CD52 is a cell-surface glycopeptide expressed by virtually all human lymphocytes, monocytes, and macrophages, a small subset of granulocytes, but not erythrocytes, platelets, or hematopoietic stem cells. CD52 is expressed on all CLL cells and indolent B-NHL cells.

According to some embodiments, the CAR T-cells of the compositions disclosed herein bind to a tumor associated antigen (TAA). For example, according to some embodiments, the scFv extracellular domain of the CAR binds to CD19 or CD20 to target B cells. According to some embodiments, the scFv extracellular domain of the CAR binds to CD20. According to some embodiments, the scFv extracellular domain of the CAR T-cell targets the CD19 antigen, and has a therapeutic effect on subjects with B-cell malignancies, ALL, Follicular lymphoma, CLL, and Lymphoma. According to some embodiments, the scFv extracellular domain of the CAR targets the CD22 antigen, and has a therapeutic effect on subjects with B-cell malignancies. According to some embodiments, the scFv extracellular domain of the CAR targets CD33, and has a therapeutic effect on subjects with AML According to some embodiments, the scFv extracellular domain of the CAR targets kappa-light chain, and has a therapeutic effect on subjects with B-cell malignancies (B-NHL, CLL). According to some embodiments, the CAR T-cells of the compositions disclosed herein bind to a tumor specific antigen (TSA).

There are a number of different types of antigens [Khan, G N et al. J. Clin. Med. (2019) 8 (2): 134, citing Coulie, P G et al. Nat. Rev. Cancer (2014) 14: 135-46], including differentiation, mutated, overexpressed, and cancer-testis antigens (CTAs), some of which have been found in AML, including antigens from mutated genes such as Nucleophosmin 1 (NPM1), DNA methyltansferase 3A (DNMT3A), Fms Related Tyrosine Kinase 3 (FLT3), and Ten-Eleven Translocation 2 (TET2) [Id., citing [Saultz, J N et al. J. Clin. Med. (2016) 5: 33]. The CTAs category includes some of the oldest and best characterized families, and although MAGE family members were not found to be expressed in presentation AML patient samples with any notable frequency [Id., citing Adams, S P et al. Leukemia (2002) 16: 2238-43], helicase antigen (HAGE) and Per ARNT SIM domain containing 1 (PASD1) antigens have been [Id., citing Id., citing Adams, S P et al. Leukemia (2002) 16: 2238-42; Guinn, B A et al. Biochem. Biohys. Res. Commun. (2005) 335: 1293-1304]. The differentiation antigens category is another large group of molecules that includes, among many others, the well-known Carcinoembryonic antigen (CEA), glycoprotein 100 (gp100), melan A/melanoma antigen recognized by T cells (MART-1), prostate specific antigen (PSA), and tyrosinase antigens, but relatively few AML antigens have come from this category. The myeloid differentiation antigen CD65 is found at low levels in the least differentiated forms of AML (MO, M1), and usually appears as CD34 disappears during normal myeloid development, reflecting the lack of differentiation in the blast cells in these disease states. The largest group are the overexpressed antigens that include human epidermal growth factor receptor 2 (ErbB-2), human telomerase reverse transcriptase (hTERT), Mucin1 (MUC1), mesothelin, PSA, prostate specific membrane antigen (PSMA), survivin, WT1, p53 and cyclin Bl. [Id.]

According to some embodiments, the extracellular antigen recognition domain is directed to CD30. CD30 expression is characteristic of the malignant Reed-Sternberg cells in Hodgkin lymphoma and is expressed in several other lymphoid malignancies, such as anaplastic large-cell lymphoma (ALCL). [Diefenbach, CSM and JP Leonard. Devel. Therapeutics (2012) 32 (1) http://doi.org/10.14694/EdBook_AM.2012.32.83].

According to some embodiments, the genetically engineered population of CAR-T cells is further genetically engineered to ectopically express Id3. (Id30^{E})

According to some embodiments, the genetically engineered population of CAR-T cells genetically engineered to ectopically express Id3^{OE} is activated in vitro in the presence of one or more cytokines. According to some embodiments, the cytokine is IL-2, IL7, IL15, IL-18, IL-21 or a combination thereof. [See, e.g., Gattinoni, L. et al. Nature Reviews Cancer (2012) 12: 671-84, at 679-80; Cieri, N. et al. Blood (2013) 121 (4): 573-84; Xu, X-J et al. Oncotarget (2016) 7 (50): 82354-68; Xu, Y et al., Blood (2014) 123 (24): 3750-9; Bachmann, M F and Oxenius, A. EMBO Rep. (2007) 8 (12): 1142-48]. Dynabeads CD3/CD28 are exemplary activating agents for mouse and human T cells (ThermoFisher Scientific). Methods to characterize the activation of the CAR-T cells are described above.

According to some embodiments, the activated genetically engineered population of CAR-T cells ectopically expressing Id3^{OE} is expanded to amplify the total number of cells to achieve a therapeutic dose. According to some embodiments, the therapeutic dose is from 1×10E6 to about 20×10E6 CAR-T cells/m² body surface area, inclusive.

Cell viability and proliferation will be confirmed by standard methods.

According to some embodiments, the CAR-T cell population ectopically expressing Id3⁰E is labeled eIther with carboxyfluorescein diacetate succinimidyl ester (CFSE, C34554; Thermo Fisher) or BD Horizon™ Violet Proliferation Dye 450 (VPD-450)(1 uM) to monitor CAR-T proliferation by dye dilution by flow cytometry.

According to some embodiments, the method comprises administering a short course of chemotherapy as described above to reduce/kill the subject's T cells prior to infusion of the CAR-T cells (lymphodepletion), which creates a favorable immune environment for T cell transplantation.

According to some embodiments, the method comprises administering to the subject by infusion a pharmaceutical composition comprising the genetically engineered population of CAR-T cells comprising ectopically expressed Id30E. wherein the human CAR-T cells engineered to ectopically express Id3 have an enhanced ability to eliminate tumors compared to a control that does not ectopically express Id3.

### Additional Agents

According to some embodiments, compositions and methods as disclosed herein can utilize combination therapy of the genetically modified CAR-T cells of the described invention with one or more additional agents.

According to some embodiments, the method further includes administering an approved immune checkpoint inhibitor at a dose standard for the cancer indication. [Wardill, H. et al. Cancers (Basel) (2023) 15(16): 4137; McClanahan, F. et al. Blood (2015) 126 (2): 203-11 (CLL); Gomez-Llobell, M. et al. Front Oncol. (2022) 12: 882531 (AML); Al-Hadidi, S A and Lee, HJ. JCO Oncology Practice (2021) 17 (2): doi.org/10.1200/OP.20.00771 (cHL)].

Immune checkpoints engage when proteins on the surface of T cells recognize and bind to partner immune checkpoint proteins on other cells, such as some tumor cells. When the checkpoint and partner proteins bind together, they send an “off” signal to the T cells, which can prevent the immune system from destroying the cancer. Immune checkpoint inhibitors can block checkpoint proteins from binding with their partner proteins, which prevents the “off” signal from being sent, allowing the T cells to kill cancer cells. Checkpoint inhibitors are not effective in all cancers.

Examples of commercially available checkpoint inhibitors include anti-PD1 (e.g., lambrolizumab/pembrolizumab (KEYTRUDA®) and nivolumab (OPDIVO®), anti-PD-L1 (Atezolizumab, TECENTRIQ®, MPDL3280A), and anti-CTLA4 (ipilimumab, YERVOY®).

According to some embodiments, the additional agent comprises rituximab (anti-CD20). According to some embodiments, the additional agent comprises alemtuzumab (anti-CD52). According to some embodiments, the additional agent comprises epratuzumab (anti-CD22). Rituximab, alemtuzumab and epratuzumab have been incorporated into ALL or CLL regimens because of their activity against certain leukemia associated antigens.

According to some embodiments, the additional agent is a PD-1 agonist. For example, Curnock [JCI Insight (2021) 6 (20): e152468] identified several single variable domain on a heavy chain (VHH) PD-1 antibodies with low nanomolar affinities and fused these to the PPI15-24 TCR to produce a panel of PD-1 agonist ImmTAAI molecules that mimic the ability of PD-L1 to facilitate the colocalization of PD-1 with the TCR complex at the target cell-T cell interface and inhibit T cell function locally. For example, PD1 agonist antibody peresolimab (Eli Lilly) is in phase III. Another example, Rosnilimab (AnaptysBio), is also a PD1 agonist.

According to some embodiments, the additional agent is alpha-1-antitrypsin (AAT). It has been demonstrated that. monotherapy with clinical-grade human AAT (hAAT) reduced circulating pro-inflammatory cytokines, diminished Graft vs Host Disease (GvHD) severity, and prolonged animal survival after experimental allogeneic bone marrow transfer (Tawara et al., Proc Natl Acad Sci USA. (2012) January 10; 109(2):564-9), incorporated herein by reference. AAT treatment reduced the expansion of alloreactive T effector cells but enhanced the recovery of T regulatory T-cells, (Tregs) thus altering the ratio of donor T effector to T regulatory cells in favor of reducing the pathological process. In vitro, AAT suppressed LPS-induced in vitro secretion of proinflammatory cytokines such as TNF-α and IL-10, enhanced the production of the anti-inflammatory cytokine IL-10, and impaired NF-κB translocation in the host dendritic cells. Marcondes, Blood. 2014 (October 30; 124(18):2881-91) incorporated herein by reference show that treatment with AAT not only ameliorated GvHD but also preserved and perhaps even enhanced the graft vs leukemia (GVL) effect.

According to some embodiments, the pharmaceutical compositions comprising chimeric antigen receptor-expressing T-cells (CAR T-cells) ectopically expressing Id3^{OE} are administered consecutively or simuntaneously with alpha-1-antitrypsin (AAT). According to some embodiments, the population of CAR T-cells and the Alpha-1-antitrypsin (AAT) are in separate compositions. According to some embodiments, AAT comprises a full length AAT or a functional fragment thereof.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Materials and Methods.

Mice and human T cells. C57BL/6 (B6, H-2b, CD45.1-CD45.2⁺), B6/SJL (H-2b, CD45.1+CD45.2−), Balb/c, B6 background Id3fl/fl and NOD/SCID/IL2Rg−/− (NSG) mice were purchased from The Jackson Laboratory. Balb/b mice were maintained in house. F1 B6 mice (H-2b, CD45.1+CD45.2+) were generated by crossing C57BL/6 with B6/SJL mice. CD4-Cre+Id3fl/fl B6 mice were bred by crossing Id3fl/fl B6 mice with CD4-Cre-expressing B6 mice. Experimental protocols were approved by the Institutional Animal Care and Use Committees of Temple University and Hackensack Meridian Health. Human T cells were isolated from buffy coat of deidentified healthy donors (New York Blood Center, NY).

Murine cell preparation and culture. Naïve CD4⁺ and CD8⁺ T cells were isolated from the spleen and LNs of age matched WT and Id3-KO mice using the Biolegend naïve CD4⁺ and CD8⁺ T cell negative selection kits. TCD-BM were prepared as described.¹ Naïve CD4⁺ T cells were stimulated with anti-CD3/CD28 conjugated microbeads (Mitenyi Biotech) and cultured in Th1 polarization medium made with IMDM medium containing 10% FBS with the supplement of 10 ng/ml IL2, 10 ng/ml IL12 and 100 g/ml anti-IL4. C-kit cells were prepared from the BM by incubating with the anti-c-kit antibody conjugated microbeads before passing through the LS columns (Mitenyl Biotech). C-kit cells were cultured in RPMI1640 containing 10% FBS in the presence of SCF, IL6 and FLT3L.

Murine GVHD models. Mice underwent bone marrow transplantation as described.²⁰ To induce GVHD, we gave 850 cGray irradiation to in Balb/c mice and 1100 cGray to Balb/b mice from an X-Ray source followed by transplantation with donor B6 (TCD-BM cells (5×10⁶), with or without adding naïve CD4⁺ and CD8⁺ T cells. Recipients were monitored for survival and clinical signs of GVHD using the scoring system that includes weight loss, posture, mobility, fur and skin impairment. Epithelial organs (skin, liver, intestine) were harvested for histological examination (Pathological Core, Fox Chase Cancer Center).

Human T cells, lentivirus and human CAR-T cell production. Buffy coat was obtained from deidentified healthy donors from NYBC. Peripheral blood mononuclear cells (PBMCs) were isolated by Ficoll (GE Life Sciences, Marlborough, MA, USA) gradient. CD4⁺ and CD8⁺ T cells were isolated with MojoSort™ Human CD4⁺ or CD8⁺ T Cell Isolation Kit (Biolegend, CA). Using codon optimization method, we synthesized CD19-4-1BB-CAR and cloned it into pLenti-EF1□-GFP (pLU) vector. HEK293T cells were transfected with lentiviral CAR plasmid, pMG2d plasmid, and psPAX2 packaging plasmid using TranslT®-LT1 Transfection Reagent (Mirus, WI) to produce lentivirus. T cells were activated with Dynabeads Human T-Activator CD3/CD28 (Gibco, NY) at a 1:1 bead-to-cell ratio for lentiviral infection.²

Induction of xeno-GVHD using human T cells. T cells were depleted from PBMCs with human CD4 Ab and CD8Ab conjugated microbeads (Miltenyi, MA) (TCD-PBMCs) and i.v. injected to NSG mice (10×10⁶ TCD-PBMCs) at day 0. Seven days later, WT or ID3 KO T cells (10×10⁶ cells/mouse, i.v.) were injected to these NSG mice to induce xeno-GVHD. Mice were monitored for survival and clinical signs of GVHD.

Induction of human leukemia in NSG mice. Raji^{TGL} cells (0.2×10⁶ cells) were injected intravenously into 8 to 12 week-old NSG mice at day 0. WT or ID3 KO CAR T cells (2.5×10⁶ cells/mouse) were injected intravenously at day 3. Leukemia growth was monitored by in vivo bioluminescence imaging. Mice were anesthetized with isoflurane and administered luciferin (150 mg/kg; Pierce) via intraperitoneal injection 10 min before imaging. Imaging was performed on IVIS Lumina X5 Imaging System (Perkin Elmer, MA).

CRISPR/Cas9 KO of ID3 in human T cells. Two single guide RNA (sgRNA) sequences targeting ID3 were synthesized by integrated DNA Technologies (IDT) (Figure s9B). CD4⁺ and CD8⁺ T cells were isolated with MojoSort™. Human CD4⁺ or CD8+ T Cell Isolation Kit (Biolegend, CA) and mixed at 1: 1 ratio in RPMI-1640 supplemented with 10 ng/mL rhIL-2, 5 ng/mL of rhIL-7 and rhIL-15 each (Shenandoah). T cells were activated with Dynabeads Human T-Activator CD3/CD28 (Gibico, NY) at a 1:1 bead-to-cell ratio. At day 2, Dynabeads were removed and cells were resuspended at 1×10⁸ cells/mL in Ingenio solution (Mirus, MA). The ribo-nucleoprotein (RNP) complexes were generated by incubating each sgRNA (5 μg per 10×10⁶ cells) individually with the Alt-R CRISPR-Cas9 nuclease V3(IDT, 10 μg per 10×10⁶ cells) for 15 min at room temperature. RNP plus 16.8 pmol of electroporation enhancer (IDT) were electroporated into the cells using a BTX830 (Harvard Apparatus BTX) at 360 V and 1 ms; this process was followed by a second electrotransfer of 5 mg of gRNA 12 to 24 hours later. Following electroporation, the cells were immediately placed in prewarmed culture media. After cultured overnight, T-cell were lentivirally transduced with CD19-CAR in some experiments and expanded to produce CD19-CAR T cells.

CRISPR/Cas9 KO editing assessment (Indel-seq analysis). Genomic DNA from WT or ID3 KO human T cells were extracted with Wizard SV Genomic DNA Purification System (Promega). PCR primers and sequencing primers were designed as described (FIG. 16B).³ KAPA HiFi HotStart PCR kit was used for target sequence amplification and used following manufacturer's protocol and MinElute Reaction Cleanup kit (Qiagen) was used for DNA purification. Analysis of gene editing efficiency was assessed by Sanger sequencing (Genewiz). Two sets of KO T cells per group were obtained: one bearing small insertions and deletions due to a single sgRNA hit, and a second population of CAR T cells bearing a large fragment deletion because of a double sgRNA hit. Synthego's Performance Analysis ICE (short for Inference of CRISPR Edits) tool was used to calculate the editing efficiency.

Retrovirus preparation and transduction. Id3 was cloned to a GFP-expressing retroviral vector.⁴ Retrovirus was prepared using Platinum-E packaging cell line (CellBiolabs). Retroviral transduction of murine C-kit+HSPCs was performed as described.⁵

Antibodies (Abs), flow cytometry analysis and cell sorting. Single-cell lymphocyte suspensions from the spleen, mLN and liver were separated by described.^{6,7} Intestine lymphocyte infiltrates were isolated as described.⁸ All antibodies used for immunofluorescence staining were purchased from eBioscience (San Diego, CA), BioLegend (San Diego, CA) or BD Biosciences (San Jose, CA). Cells were stained with appropriate concentrations of mAbs. Dead cells were excluded using Fixable Viability Dye from eBioscience (San Diego, CA). Flow cytometry analyses were performed using BD LSRII, LSRFortessa or FACSymphony A3 cytometers (BD Bioscience, CA). Cell sorting was performed with BD Aria (BD Bioscience, CA).

Real-time quantitative PCR. Total RNA was extracted from cultured or sorted T cells with Trizol (Invitrogen Life Technologies), digested with DNase to remove genomic DNA and reverse transcribed into cDNA using Superscript IV VILO cDNA synthesis kit (Invitrogen Life Technologies). Real-time quantitative PCR was performed with the SYBR Green PCR master mix with ROX as the reference dye (Roche) on the QuantStudio 5 Real-time PCR instrument. Gene expression level was calculated relative to the endogenous control 18S rRNA.

Bulk RNA sequencing and analysis. Total RNA was extracted with Qiagen RNeasy Mini Kit. Directional mRNA libraries were prepared using the poly(A) enrichment pipeline with the NEBNext Ultra II Directional RNA Library Prep Kit for Illumina (New England Biolabs, Inc) following the manufacturer's protocol. Libraries were sequenced on Novaseq in paired-end mode with the read length of 150 nucleotides by Novogene. Transcript-level quantification was performed with Salmon v1.9.0 against the GENCODE GRCm39 v27 transcriptome.⁹ Differential expression was assessed using the ‘treat’ function from the ‘limma’ package by fitting gene-wise linear models on the log 2-qsmooth-normalized counts and testing against a log-fold change of log 2(1.2). Principal component biplots were produced by performing PCA on the normalized log counts with the ‘pca’ function from the ‘PCA tools’ package. Gene set enrichment analysis was performed using the ‘fgsea’ package by testing for enrichment against the MSigDb v7.4 HALLMARK gene sets for mouse. Gene ontology analysis was performed using the ‘enrich GO’ function from the ‘cluster Profiler’ R package.

ATAC-seq and data analysis. Biological triplicates of 25,000 CD4⁺ T cells stimulated for 24 hours and sorted 4-day cultured CD62L⁻CD44⁺, CD62L⁺CD44⁺Th1 cells were collected for ATAC-seq library preparation following a published protocol.¹⁰ Briefly, cells were lysed for 3 minutes in ATAC-Resuspension Buffer containing 0.1% NP40, 0.1% Tween-20 and 0.01% Digitonin for nuclei extraction. Nuclei were washed, pelleted and resuspended in 25 ul transposition mix (Illumina) for transposition reaction at 37° C. for 25 minutes. Transposed DNA fragments were cleaned up with the MinElute Reaction Cleanup kit (Qiagen), amplified by PCR for 12 cycles with barcoded Nextera primers (Illumina). PCR products were run on 2% E-Gel EX Agarose Gels (Invitrogen) to extract DNA fragments in the range of 150-600 bp using a Gel Extraction kit (Qiagen). The libraries were quantified using the KAPA Library Quantification kit with fragment size normalized using the Bioanalyzer (Agilent). Paired-end sequencing with a read length of 150 bp was performed on Illumina HiSeq4000 at Admera Health. Paired-end ATAC-seq reads were trimmed with trim galore.¹¹ BAM files were then imported into ‘R’ and 300 bp window counts were generated using the ‘windows Counts’ function from the ‘csaw’ package. ENCODE blacklist regions were excluded from window counting. Local windows were then filtered for background enrichment greater than log 2. 10,000 bp background window counts were used to generate scaling factors for TMM normalization. Differential accessibility of local windows was then assessed with ‘edgeR’ using the ‘glmQLFTest’ function. The ‘merge Results’ function from the ‘csaw’ package was then used to merge window-level statistics to find regions of differential accessibility. Motif analysis was performed by extracting genomic sequences from regions of differential accessibility (FDR<0.05) that overlapped the upstream regions of differentially expressed genes (FDR<0.05). De novo motif analysis was then performed on these regions using STREME from the MEME suite v5.4.1. De novo motifs were then assessed for enrichment of known transcription factor binding motifs in the HOCOMOCOv11_full_MOUSE database using TOMTOM from the MEME suite.

Single cell RNA sequencing (scRNA) analysis. Donor CD4⁺ T cells were enriched from the spleen and liver of Balb/c recipients 21 days after HSCT using positive selection beads (Mitenyi Biotech). Same numbers of T cells from different mice of the same group were pooled, generating 4 samples for scRNA-seq library preparation: WT spleen, WT liver, Id3 spleen, Id3 liver. 10,000 cells from each sample were loaded onto the Chromium Chip B to generate single-cell gel beads-in-emulsion (GEM) for a target recovery of 6,000 single cells. mRNA from single cells were reverse transcribed, barcoded and amplified via PCR for library construction. scRNA-seq libraries were generated using Chromium™ Single Cell 3′ GEM, Library & Gel Bead Kit v3 following the manufacturer's manual (10× Genomics). Each library was loaded to a lane of an Illumina HiSeq4000 sequencer for paired-end sequencing with a read length of 150 bp and a depth of 350M reads per library (Genewiz). Per cell feature counts for sequenced libraries were generated using 10× Genomics Cell Ranger 3.1.0 by aligning to the cellranger-mm10-3.0.0 reference transcriptorne.¹² The resulting filtered feature barcode matrices for each sample were imported into R using the ‘Read10×’ function from the Seurat V3 package. Quality control was performed by removing cells with low feature counts (<200 unique genes) and cells with a mitochondrial percentage greater than 5%. After removing low quality cells, the data was log-normalized and the 2,000 most variable features were selected for each sample using the ‘Find Variable Features’ function in the Seurat package. The data were then scaled and dimensionality reduction was performed using the ‘run PCA’ function. The optimal number of principal components to keep was determined using the Jack Straw method. Graph-based clustering was then performed by building the nearest neighbor graph with ‘Find Neighbors’ and clustering with ‘Find Clusters’. Visualization of dimensionality reduction and clustering was performed with the ‘run UMAP’ and ‘Dim Plot’ functions. Differentially expressed markers per-cluster were assessed using the ‘Find All Markers’ function using a log-fold-change threshold of 0.25 and a minimum percentage expressed of 5%. The average expression value for each gene was calculated by taking the natural logarithm of the average count after adding a pseudo count of 1. Heatmaps were constructed from these expression values.

Data sharing statement. For information and requests for resources and reagents, please contact Yi Zhang (yi.zhang@hmh-cdi.org). The GEO ids for each of the experiments are available as the following: Super Series Accession: GSE230286, RNA-seq data: GSE230274, scRNA-seq data: GSE230285, and ATAC-seq data: GSE230082.

Statistical analysis. Survival differences between groups were determined with log-rank test. Clinical scores were compared between groups using Student's t test and Mann-Whitney test. Two-group means comparisons were performed with Student's t test; multiple-group means comparisons were performed with One-Way ANOVA. p-value less than 0.05 is considered as significant difference.

### REFERENCES

1. Zhang, Y., Joe, G., Hexner, E., Zhu, J. & Emerson, S. G. Host-reactive CD8⁺ memory stem cells in graft-versus-host disease. Nat Med 11, 1299-1305 (2005).
2. Porter, D. L., Levine, B. L., Kalos, M., Bagg, A. & June, C. H. Chimeric antigen receptor-modified T cells in chronic lymphoid leukemia. New England Journal of Medicine 365, 725-733 (2011).
3. Good, C. R., et al. An NK-like CAR T cell transition in CAR T cell dysfunction. Cell 184, 6081-6100 e6026 (2021).
4. He, S., et al. Ezh2 phosphorylation state determines its capacity to maintain CD8(+) T memory precursors for antitumor immunity. Nat Commun 8, 2125 (2017).
5. Zhu, J., Zhang, Y., Joe, G. J., Pompetti, R. & Emerson, S. G. NF-Ya activates multiple hematopoietic stem cell (HSC) regulatory genes and promotes HSC self-renewal. Proc Natl Acad Sci USA 102, 11728-11733 (2005).
6. Zhang, Y., et al. Notch signaling is a critical regulator of allogeneic CD4⁺ T-cell responses mediating graft-versus-host disease. Blood 117, 299-308 (2011).
7. Yu, X., et al. A Novel RIPK1 Inhibitor Reduces GVHD in Mice via a Non-immunosuppressive Mechanism that Restores Intestinal Homeostasis. Blood (2022).
8. Qiu, Z. & Sheridan, B. S. Isolating Lymphocytes from the Mouse Small Intestinal Immune System. J Vis Exp (2018).
9. Patro, R., Duggal, G., Love, M. I., Irizarry, R. A. & Kingsford, C. Salmon provides fast and bias-aware quantification of transcript expression. Nat Methods 14, 417-419 (2017).
10. Corces, M. R., et al. An improved ATAC-seq protocol reduces background and enables interrogation of frozen tissues. Nat Methods 14, 959-962 (2017).
11. Lun, A. T. & Smyth, G. K. csaw: a Bioconductor package for differential binding analysis of ChIP-seq data using sliding windows. Nucleic Acids Res 44, e45 (2016).
12. Stuart, T., et al. Comprehensive Integration of Single-Cell Data. Cell 177, 1888-1902 e1821 (2019).

### Results

T cells require Id3 to maintain GVHD in peripheral tissues. To investigate a role of Id3 in T cell-mediated GVHD, we isolated CD4⁺ T cells from wild-type (WT) and CD4-Cre⁺Id3^{fl/fl} B6 mice (Id3-cKO), mixed with T cell-depleted bone marrow (TCD-BM) cells from B6(F1) mice, and transplanted them into lethally irradiated Balb/c mice. WT T cells caused severe GVHD, with 90% of them succumbing to the disease (FIG. 1A, FIG. s1A). Id3-cKO T cells failed to induce lethal GVHD, with all recipients surviving during the observation period (FIG. 1A, FIG. s1A). This was accompanied by absence of cutaneous GVHD (FIG. 1B, FIG. s1B-C), and reduced inflammation in the intestine (FIG. 1C), skin and liver (FIG. s1B-C).

To understand the underlying mechanism, we determined whether Id3 ablation impaired engraftment, proliferation and migration of donor T cells. By days 8 and 22 after transplantation, Id3 loss did not significantly reduce the frequency and number of IFN-γ-producing CD4+Th1 cells in the spleen and mesenteric lymph node (mLN) (FIG. 1D, FIG. 1E), but profoundly diminished Th1 cells in the liver (3- and 2.5-fold, respectively, FIG. 1E). Id3 loss also decreased numbers of IFN-γ-producing Th1 effectors in the intestine 17 days after transplantation (FIG. 1F). BrdU incorporation assay showed that Id3 loss did not decrease donor T cell proliferation (FIG. 8D). Chemokine receptors CCR9 and CCR5 and adhesion molecule α4β7 are required for T cell migration into GVHD tissues.^{20,21} Id3 ablation did not reduce the expression of CCR5 and α4β7 on the surface of alloreactive T cells in the mLN (FIG. 1G, FIG. 8E). Instead, alloreactive Id3-cKO CD4+ T cells produced higher frequency of CCR9+ effectors (FIG. 1G, FIG. 8E). Thus, Id3 maintains alloreactive Th1 cells in GVHD target tissues via a mechanism independent of proliferation and tissue migration.

Id3 represses genes that regulate effector differentiation and expression of inhibitory receptors. To identify the Id3-targeted genes associated with alloreactive T cell function, we performed RNA-seq analysis. Effector differentiation is associated with downregulation of Id3 expression.^{15,18} Ninety-six hours after culture under Th1 cell condition, both WT and Id3-cKO CD4+ T cells upregulated CD44 but the latter showed more profound downregulation of CD62L (FIG. 9A). To avoid the potential contribution of different Th1 cell subsets to Id3 deficiency-mediated gene expression, we performed RNA-seq analysis using mRNA from highly purified CD44hiCD62Llo and CD44hiCD62Lhi Th1 cells (FIG. 9A). Additionally, to capture the earliest molecular changes of Id3 deficiency upon Th1 induction, we analyzed gene profiles of CD4+ T cells 24 hours after TCR activation (TCR-primed CD4+ T cells) (FIG. 2B). TCR-priming induced significant transcriptional diversification between WT and Id3-cKO CD4+ T cells. This diversification was dramatically amplified between WT and Id3-cKO CD44hiCD62Llo Th1 cells (FIG. 9B).

Without Id3, TCR-primed CD4+ T cells primarily induced gene expression (FIG. 9C). These differentially expressed genes (DEGs) were associated with activation of ‘TNFA signaling via NFκB, Apoptosis and IL-6/JAK/STAT3 signaling’ (FIG. 9D). Upon Th1 cell differentiation, Id3 deficiency induced 4265 genes and 4150 genes, but repressed 260 genes and 476 genes, in CD44^{hi}CD62L^{lo} Th1 cells and CD44^{hi}CD62L^{hi} Th1 cells, respectively (FIG. 9C). However, only few DEGs were identified between CD44^{hi}CD62L^{lo} and CD44^{hi}CD62L^{hi} Th1 cells despite Id3 ablation (FIG. 9E). Thus, CD62L expression did not distinguish Id3-controlled transcription in Th1 cells. We therefore focused on analyzing DEGs in CD44^{hi}CD62L^{lo} Th1 cells. Functional annotation of these DEGs indicated strong enrichment of ‘IL-2-STAT5 signaling’, ‘IFN-γ response’ and ‘allograft rejection’ besides those identified in TCR-primed CD4+ T cells (FIG. 2A-B). Thus, Id3 is important for repressing genes critical for effector differentiation and IFN responses but promoting cell survival.

We further characterized those Id3-repressed gene pathways with higher GSEA enrichment scores (FIG. 9C, FIG. 2A), which represented the transcriptional signature repressed by Id3 in TCR-primed T cells and Th1 cells (FIG. 2B, Table 1).

TABLE 1
Transcriptional signature repressed by Id3 in TCR-primed T cells and Th2 cells.

| Contrast | Pathway | PVal. | Padj. | Log2err. | ES | NES | Size | Leading Edge |
|---|---|---|---|---|---|---|---|---|
| ID3 v | Wang- | 0.000246 | 0.000246 | 0.311078 | 0.77902 | 1.502782 | 96 | Il13 Il5 |
| WT.TCR | Id3 | | | | | | | Entpd1 Il4 |
| prid | TCR | | | | | | | Il31 Il24 Il17f |
| | primed | | | | | | | Id2 Runx2 |
| | | | | | | | | Il10 Il17a |
| | | | | | | | | Tigit Haver2 |
| | | | | | | | | Casp6 Gata3 |
| | | | | | | | | Il21 Il1a |
| | | | | | | | | Tnfrsf11b |
| | | | | | | | | Tnfsf10 Batf |
| | | | | | | | | Il6 Prdm1 |
| | | | | | | | | Tnfrsf8 Fosl1 |
| | | | | | | | | Tnfrsf9 Fos |
| | | | | | | | | Il17re Atf3 |
| | | | | | | | | Il17rb Casp1 |
| | | | | | | | | Tnfsf8 |
| | | | | | | | | Tnfsf14 Fosb |
| | | | | | | | | Bhlhe40 Rorc |
| | | | | | | | | Il23a Tox2 |
| | | | | | | | | Tnfsf9 Nfkb2 |
| | | | | | | | | Tnfrsf12a |
| | | | | | | | | Nfkbia Il18 |
| | | | | | | | | Egr1 Il9r |
| | | | | | | | | Nr4a2 |
| ID6_V_ | Wang- | 1.98E−06 | 1.98E−06 | 0.627257 | 0.6509 | 1.424667 | 96 | Gata3 Batf |
| WT.Th1 | Id3-Th1 | | | | | | | Irf4 Nfkbia |
| effectors | effector | | | | | | | Tnfrsf4 Cd274 |
| | cells | | | | | | | Irf1 Il24 Irf9 |
| | | | | | | | | Nfat5 Ets1 |
| | | | | | | | | Il12rb1 Junb |
| | | | | | | | | Il27ra Il4ra |
| | | | | | | | | Ripk1 Nfkb2 |
| | | | | | | | | Stat2 Il13 |
| | | | | | | | | Il17a |
| | | | | | | | | Tnfrsf12a |
| | | | | | | | | Ifngr2 Atf6 |
| | | | | | | | | Jun Stat3 |
| | | | | | | | | Notch1 Tox4 |
| | | | | | | | | Mcl1 Id2 Il5 |
| | | | | | | | | Tnfrsf8 |
| | | | | | | | | Tnfsf13b Il21 |
| | | | | | | | | Rel Il31 Jund |
| | | | | | | | | Il15ra Il3ra |
| | | | | | | | | Casp2 Casp6 |
| | | | | | | | | Il6 Entpd1 |
| | | | | | | | | Rorc Icos |
| | | | | | | | | Tnfsf10 |
| | | | | | | | | Gsdmd |
| | | | | | | | | Havcr2 |
| | | | | | | | | Tnfrsf18 |
| | | | | | | | | Bhlhe40 |
| | | | | | | | | Stat5a Atf3 |
| | | | | | | | | Tigit Il17re |
| | | | | | | | | Stat1 |
| | | | | | | | | Tnfrsf13b |
| | | | | | | | | Prdm1 Ctla4 |
| | | | | | | | | Il4 Tox2 |
| | | | | | | | | Runx1 Ripk3 |
| | | | | | | | | Lag3 Nfatc2 |
| | | | | | | | | Zbp1 Cd28 |
| | | | | | | | | Egr1 |
| | | | | | | | | Casp8ap2 |
| | | | | | | | | Il17rb Tnfrsf9 |
| | | | | | | | | Il6ra Tnfsf9 |
| | | | | | | | | Fosb Il18 |
| | | | | | | | | Casp1 Il17f |
| | | | | | | | | Fosl1 Il9r |
| | | | | | | | | Il23a Hey1 |
| | | | | | | | | Bad Tnfsf8 |
| | | | | | | | | Il10 Il1a |

We classified them into 5 clusters, including: i) TFs critical for effector proliferation and differentiation; ii) cytokines that distinguish Th1 from other lineages (IL-4, IL-5, IL-13, IL-17a, IL-17f and IL21); iii) cell survival and death molecules (e.g., Mcll, Bcl2111, Bad, Zbp1); iv) costimulatory molecules important for GVHD T cell proliferation and survival (e.g., Cd28, Icos, Tnfsf8, Tnfrsf9); and v) inhibitory receptors (FIG. 2C-G). Id3 loss upregulated expression of TF genes that promote Th1 (Atf3), Th17 (Batf) and Th2 cell (Gata3) differentiation (FIG. 2C). Bhlhe40, which promotes generation of GM-CSF-secreting effector T cells,⁹ was highly increased Id3-cKO Th1 cells (FIG. 2C). Enhanced gene transcripts encoding Th2 and Th17 cytokines agrees with previous observations that Id3 represses Th2- and Th17-cell differentiation.^{11,22,23} Id3 inhibition led to marked upregulation of inhibitory receptors (e.g., Pdcdl, Cd274, Ctla4, Havcr2 and Entpd1) (FIG. 2G). We verified that Id3 loss caused significant increases of PD-1+ and PD-L1(CD274)+Th1 cells (FIG. 2H-J). These results reveal a previously unrecognized role of Id3 in limiting expression of gene programs that promote effector differentiation and expression of immune inhibitory PD-1 and PD-L1.

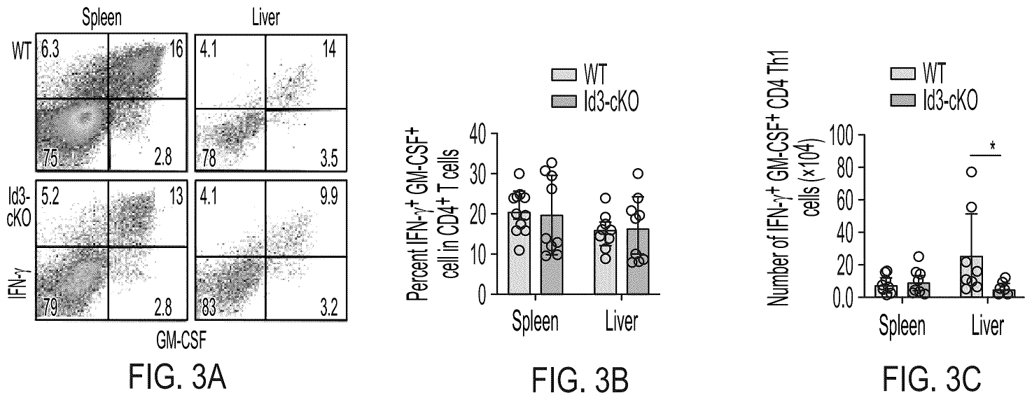

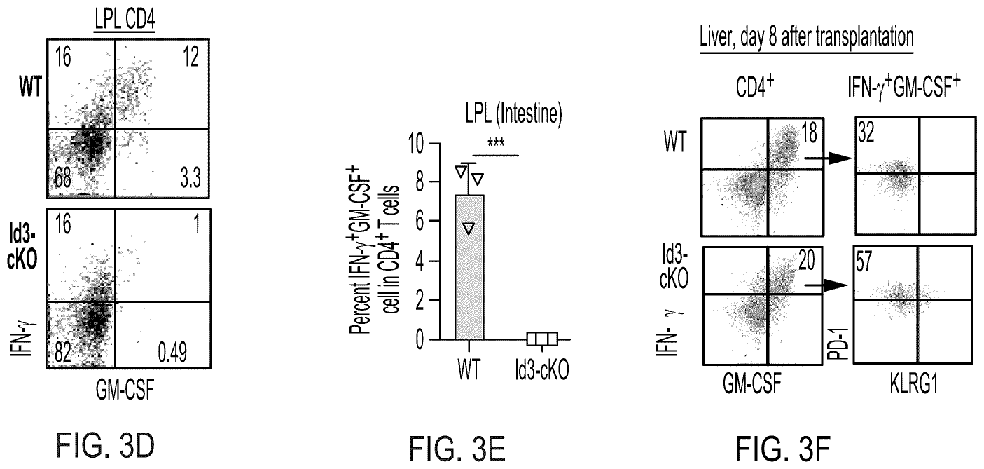

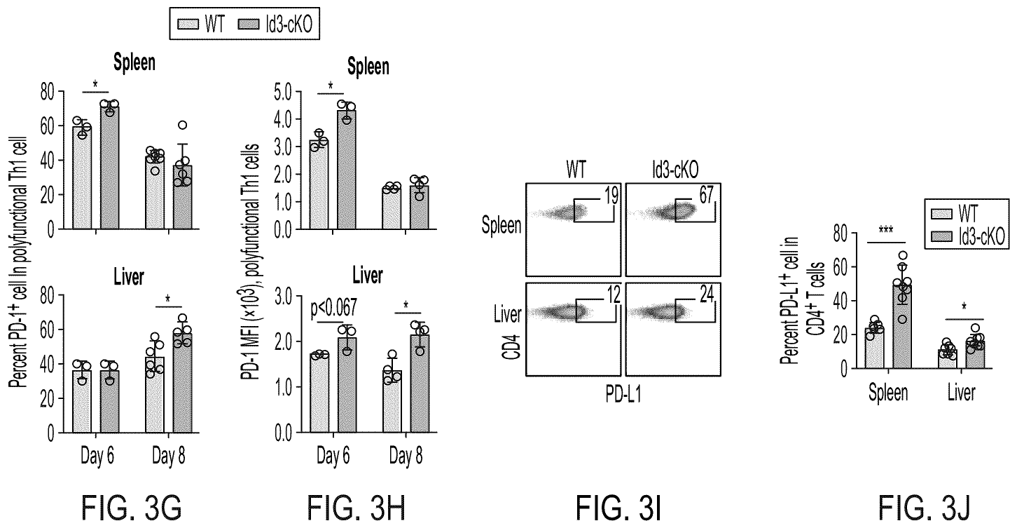

Id3 limits PD-1 expression by tissue-infiltrating alloreactive Th1 cells. Activation of PD-1 signaling by PD-L1 causes T cell death and dysfunction, thereby limiting T cell-mediated tissue injury.²⁴⁻²⁶ To test the impact of Id3 deficiency on PD-1 expression in alloreactive T cells, we recovered donor T cells from Balb/c mice 6 and 8 days after allo-HSCT. Alloantigen-reactive effector T cells that simultaneously produce IFN-γ and GM-CSF are responsible for mediating severe GVHD and enriched for alloantigen-specific T cells.^{7,27,28} Compared to WT CD4+ T cells, Id3-cKO CD4+ T cells produced similar frequencies of IFN-γ+GM-CSF+Th1 cells (resembling polyfunctional Th1 cells) in the spleen and liver 8 days after transplantation (FIG. 3A-B), but approximately 5-fold fewer polyfunctional Th1 cell numbers in the liver (FIG. 3C). Id3 loss also led to approximately 10-fold fewer polyfunctional Th1 cells in the intestine 17 days post-HSCT (FIG. 3D-E). Thus, Id3 is critical for sustaining alloreactive Th1 cells that infiltrate GVHD target tissues. Further analysis revealed that Id3 ablation significantly increased the frequency of PD-1+ polyfunctional Th1 CD4+ cells and their PD-1 protein levels (mean fluorescence intensity, MFI) initially in the spleen 6 days after transplantation and subsequently in the liver by 8 days (FIG. 3F-H). Id3 deficiency resulted in significantly increased percentage of PD-L1-expressing Th1 cells (FIG. 3I-J). These data verify the importance of Id3 in repressing expression of PD-1 and PD-L1 by alloreactive effector T cells during GVHD.

PD-1 blockade restores the capability of Id3-cKO T cells to mediate GVHD. Activating PD-1 in donor T cells is important for reducing GVHD.29-32 We examined the impact of the PD-1/PD-L1 pathway on Id3 deficiency-mediated GVHD inhibition using two different mouse models. First, we used the B6 anti-Balb/b mouse model of GVHD directed against minor histocompatibility antigen (miHA), in which both donor CD4+ and CD8+ T cells induce lethal GVHD.33 Again, Id3-cKO T cells failed to induce severe GVHD in Balb/b recipients (FIG. 4A). However, anti-PD-1 Ab treatment induced severe GVHD in Id3-cKO T cell Balb/b recipients, with 80% of them dying from the disease (FIG. 4A). Compared to saline control, PD-1 blockade resulted in significantly increased numbers of donor Id3-cKO CD4+ T cells in the liver and intestine (FIG. 4B), and increased frequency of PD-1+CD4+ T cells in the spleen and liver (FIG. 4C). Alloreactive Id3-cKO CD4+ T cells derived from these PD-1 Ab-treated mice expressed lower levels of PD-1 than their WT counterparts (FIG. 4D). This coincides with previous observations that PD-1 blockade reinvigorates tumor-reactive T cells expressing low levels of PD-1.^{25,34} Notably, although PD-1 blockade did not affect the frequency of polyfunctional Th1 cells in the liver (FIG. 10A-B), it caused a 1.5-fold increase of polyfunctional Th1 cell numbers compared to saline control (FIG. 4E), suggesting improved survival of liver-infiltrating Id3-cKO Th1 cells.

We validated this conclusion using MHC- and miHA-mismatched B6 anti-Balb/c mouse GVHD model. Compared to saline treatment, PD-1 blockade induced more severe GVHD in Id3-cKO T cell recipients, with approximately 40% mortality by day 65 (FIG. 3F, FIG. 13C). PD-1 blockade increased by 2-fold polyfunctional Th1 cells in the liver of mice receiving Id3-cKO T cells compared to saline treatment (FIG. 10D).

In the B6 anti-Balb/b mouse model, miHA H60-specific (H60+) CD8+ T cells are responsible for inducing GVHD.^{33,35} Id3-cKO CD8+ T cells produced 2-fold lower frequency of H60+CD8+ T cells and 10-fold lower PD-1′H60+CD8+ T cell numbers compared to WT CD8+ T cells (FIG. 11A, 11B, 11C). PD-1 Ab treatment increased the fraction of both H60+CD8+ T cells and PD-1′H60+CD8+ T cells compared to saline control (FIG. 11B, 11C). Taken together, our findings demonstrate that Id3 is required to protect tissue-infiltrating alloreactive CD4+ and CD8+ T cells from PD-1-mediated suppression.

Id3 maintains tissue-infiltrating PD-1+TCF-1+ progenitor-like T cells and polyfunctional PD-1+Th1 cells. We examined the mechanism by which Id3 maintained tissue-infiltrating Th1 cells. Id3 is required for TCF-1-mediated recall response of memory CD8+ T cells.¹⁷ During GVHD, tissue-resident TCF-1+ progenitor-like CD4⁺ T cells are associated with alloreactive effector T cell persistence.^{20,10} To test whether Id3 deficiency may reduce tissue-infiltrating PD-1+TCF-1+ progenitor-like T cells, we purified PD-1′KLRG-1-donor T cells from the spleen, mLN, and liver of Balb/c mice receiving CD4+ T cells derived from WT and Id3-cKO B6 mice 8 days after transplantation, and transferred them into lethally irradiated secondary Balb/c recipients (FIG. 12A). Compared to their WT counterparts, Id3-cKO PD-1′KLRG-1− effectors generated 2.5-fold lower frequency of liver-infiltrating PD-1′KLRG1-Th1 cells (FIG. 12B, 12C). Meanwhile, they failed to produce PD-1′TCF-1+ progenitor-like T cells in the liver (FIG. 12D). In contrast, ectopic expression of Id3 in Id3-cKO T cells produced 4.6-fold more PD-1+ polyfunctional Th1 cells in the liver compared to GFP-expressing Id3-cKO CD4+ T cells (FIG. 12E). Thus, Id3 is important for maintaining tissue-infiltrating PD-1+TCF-1+ progenitor-like T cells and PD-1+Th1 cells.

Id3 ablation leads to PD-1-mediated depletion of tissue-infiltrating Th1 cells. To define the mechanism through which Id3 maintains liver-infiltrating polyfunctional Th1 cells and progenitor-like cells, we performed scRNA-seq analysis using alloreactive CD4+ T cells isolated from the liver of Balb/c mice receiving WT and Id3-cKO B6 T cells. We identified 6 clusters of donor T cells in the liver, and among them were 3 clusters of Th1 cells expressing Ifng (FIG. 5A-B; FIG. 13A). WT cluster 1A and cluster 3B cells had higher frequency of Csf2 (encodes GM-CSF)-expressing cells than cluster 3A (FIG. 5B), suggesting that they were enriched for IFN-γ+GM-CSF+Th1 cells. Both WT cluster C1A and C3B cells expressed Pdcdl transcript, with greater abundance in C3B (FIG. 5C). Id3 loss resulted in upregulated Csf2 gene expression in cluster C1A and C3B cells, and increases of Pdcdl-expressing cells in cluster C3B cells (FIG. 5C), and decreased frequency of C3B cells (FIG. 13B). While WT C7 cluster cells were characterized by Tcf7 (encoding TCF-1) expression, Id3-cKO C7 cluster cells were depleted of Tcf7 (FIG. 13A) and had 4-fold fewer cells in frequency compared to their WT counterparts (FIG. 13B). Thus, Id3 is required for maintaining TCF-1+ progenitor-like cells and limiting the generation of PD-1+ polyfunctional Th1 cells in the local tissue.

Activation of PD-1 inhibits T cell responses primarily by inactivating CD28 signaling and/or TCR signaling^{24,25,36} and PJ3K/Akt pathway.^{27,34} We found that C1A cells, enriched in polyfunctional Th1 cells, were a dominant cell subset in both WT and Id3-cKO compared to C3B3 and C7 (FIG. 13B). WT C1A cluster was characterized with gene programs associated with highly active TNFR2-signaling, CD28 costimulatory signaling and PKC-θ signaling activity (FIG. 5D)). These WI C1A cells expressed molecular features associated with tissue invasiveness and residence (Ccl1, Ccr2, Cxcr6, Ccr12, Itgb1, Table 2), resembling those tissue resident GVHD T cells,^{1,2} suggesting their crucial roles in tissue injury.

TABLE 2
Tissue invasiveness-and residence-associated molecules expressed by WT C1A cells.

| Gene Name | P Val. | Ave Log FC | Pct.1 | Pct.2 | P_Val_Adj. | Cluster |
|---|---|---|---|---|---|---|
| Ifitm1 | 1.30E−16 | 2.11465402 | 0.17 | 0 | 4.03E−12 | 1A |
| Lgals3 | 1.86E−18 | 1.06524734 | 0.352 | 0.097 | 5.77E−14 | 1A |
| S100a6 | 1.52E−45 | 1.01718565 | 0.843 | 0.458 | 4.72E−41 | 1A |
| Ccl1 | 2.16E−09 | 1.00413911 | 0.197 | 0.058 | 6.70E−05 | 1A |
| Ifitm2 | 9.69E−28 | 0.97550419 | 0.537 | 0.169 | 3.01E−23 | 1A |
| Atf3 | 2.48E−16 | 0.95928364 | 0.33 | 0.097 | 7.69E−12 | 1A |
| Areg | 1.17E−07 | 0.89637347 | 0.105 | 0.014 | 0.00364306 | 1A |
| Ifitm3 | 1.84E−14 | 0.89169092 | 0.317 | 0.103 | 5.71E−10 | 1A |
| AA467197 | 1.56E−08 | 0.86464808 | 0.107 | 0.011 | 4.83E−04 | 1A |
| Csf2 | 8.99E−09 | 0.86395079 | 0.205 | 0.067 | 2.79E−04 | 1A |
| Prr7 | 1.23E−09 | 0.81299222 | 0.178 | 0.044 | 3.82E−05 | 1A |
| Gata3 | 2.35E−12 | 0.8055344 | 0.327 | 0.128 | 7.31E−08 | 1A |
| Crip1 | 4.77E−30 | 0.80424878 | 0.825 | 0.519 | 1.48E−25 | 1A |
| Hilpda | 3.96E−14 | 0.76066838 | 0.461 | 0.217 | 1.23E−09 | 1A |
| Vim | 5.54E−20 | 0.72401743 | 0.617 | 0.308 | 1.72E−15 | 1A |
| Lgals1 | 2.48E−24 | 0.72281643 | 0.702 | 0.35 | 7.71E−20 | 1A |
| Rgcc | 3.45E−07 | 0.65900347 | 0.197 | 0.075 | 0.0107063 | 1A |
| Hk2 | 7.34E−05 | 0.58252441 | 0.144 | 0.061 | 1 | 1A |
| Capg | 5.67E−09 | 0.58038837 | 0.19 | 0.053 | 1.76E−04 | 1A |
| Nfkbia | 1.25E−24 | 0.57316065 | 0.873 | 0.592 | 3.89E−20 | 1A |
| S100a4 | 2.57E−18 | 0.57004991 | 0.704 | 0.403 | 8.00E−14 | 1A |
| Ahnak | 3.99E−15 | 0.56763439 | 0.624 | 0.333 | 1.24E−10 | 1A |
| Pim1 | 8.93E−18 | 0.55571152 | 0.74 | 0.464 | 2.77E−13 | 1A |
| Hspa1a | 5.30E−04 | 0.55098665 | 0.163 | 0.086 | 1 | 1A |
| Kdm6b | 1.77E−08 | 0.53060196 | 0.412 | 0.231 | 5.50E−04 | 1A |
| Rora | 2.57E−06 | 0.5251306 | 0.182 | 0.072 | 0.0797903 | 1A |
| Tnfsf8 | 2.10E−05 | 0.5077615 | 0.263 | 0.147 | 0.65103046 | 1A |
| Tagln2 | 1.65E−08 | 0.50747168 | 0.31 | 0.142 | 5.13E−04 | 1A |
| Cish | 2.50E−05 | 0.49784997 | 0.218 | 0.111 | 0.77787338 | 1A |
| Map2k3 | 8.11E−05 | 0.49081602 | 0.12 | 0.044 | 1 | 1A |
| Nfkb1 | 2.99E−08 | 0.48177652 | 0.342 | 0.172 | 9.29E−04 | 1A |
| Rel | 9.78E−07 | 0.47645022 | 0.207 | 0.083 | 0.03036356 | 1A |
| Crybg1 | 1.62E−04 | 0.46408961 | 0.103 | 0.036 | 1 | 1A |
| Fosl2 | 2.01E−04 | 0.45488904 | 0.134 | 0.058 | 1 | 1A |
| Rgs2 | 6.42E−10 | 0.4529133 | 0.479 | 0.256 | 1.99E−05 | 1A |
| Zyx | 1.52E−04 | 0.45158485 | 0.16 | 0.075 | 1 | 1A |
| Nfkbiz | 2.37E−06 | 0.44959523 | 0.296 | 0.153 | 0.07351028 | 1A |
| Itgb1 | 6.72E−09 | 0.44729496 | 0.395 | 0.197 | 2.09E−04 | 1A |
| Hsph1 | 4.71E−04 | 0.44580451 | 0.113 | 0.047 | 1 | 1A |
| Cd401g | 8.32E−05 | 0.44264573 | 0.228 | 0.125 | 1 | 1A |
| Lilr4b | 2.78E−05 | 0.43775204 | 0.43 | 0.294 | 0.86421015 | 1A |
| Ccrl2 | 2.94E−04 | 0.42863233 | 0.109 | 0.042 | 1 | 1A |
| Dnajc15 | 6.27E−04 | 0.42270557 | 0.195 | 0.114 | 1 | 1A |
| Map3k8 | 0.0014717 | 0.41915769 | 0.116 | 0.056 | 1 | 1A |
| Emp3 | 4.93E−06 | 0.41286238 | 0.28 | 0.144 | 0.15299101 | 1A |
| Fmnl1 | 1.49E−04 | 0.40434805 | 0.177 | 0.086 | 1 | 1A |
| Ctla2a | 0.00133638 | 0.40265394 | 0.14 | 0.072 | 1 | 1A |
| Itgb7 | 1.79E−06 | 0.39914369 | 0.25 | 0.117 | 0.05551209 | 1A |
| Sytl3 | 0.00107179 | 0.39531649 | 0.112 | 0.05 | 1 | 1A |
| Chd7 | 3.65E−04 | 0.3922145 | 0.235 | 0.139 | 1 | 1A |
| Traf1 | 1.60E−07 | 0.39202313 | 0.386 | 0.211 | 0.00496677 | 1A |
| Sqstm1 | 1.18E−04 | 0.38567984 | 0.211 | 0.114 | 1 | 1A |
| Nr4a1 | 1.11E−05 | 0.38532157 | 0.347 | 0.203 | 0.34337165 | 1A |
| S100a10 | 2.30E−10 | 0.38507457 | 0.76 | 0.575 | 7.15E−06 | 1A |
| Ppp2r5a | 8.41E−04 | 0.38199065 | 0.11 | 0.047 | 1 | 1A |
| Cxcr6 | 0.00125456 | 0.37637675 | 0.197 | 0.117 | 1 | 1A |
| Ifng | 3.57E−04 | 0.37624714 | 0.273 | 0.172 | 1 | 1A |
| Ccr2 | 0.00265996 | 0.37221209 | 0.212 | 0.136 | 1 | 1A |
| Dennd4a | 1.33E−04 | 0.37178543 | 0.279 | 0.167 | 1 | 1A |
| Il18rap | 0.00164536 | 0.37046973 | 0.106 | 0.047 | 1 | 1A |
| Sdf4 | 3.69E−04 | 0.37032804 | 0.154 | 0.075 | 1 | 1A |
| Rasgrp2 | 0.00131115 | 0.36400135 | 0.115 | 0.053 | 1 | 1A |
| Cdkn1a | 1.13E−04 | 0.36159063 | 0.306 | 0.183 | 1 | 1A |
| Pim3 | 0.00156202 | 0.35709559 | 0.177 | 0.1 | 1 | 1A |
| Ppib | 9.51E−05 | 0.35707625 | 0.293 | 0.178 | 1 | 1A |
| Furin | 2.06E−04 | 0.35660516 | 0.27 | 0.164 | 1 | 1A |
| Cd44 | 8.96E−04 | 0.34690382 | 0.147 | 0.075 | 1 | 1A |
| Srgn | 4.75E−07 | 0.34281992 | 0.717 | 0.558 | 0.0147391 | 1A |
| Pak2 | 1.96E−04 | 0.342228 | 0.177 | 0.086 | 1 | 1A |
| Zc3hav1 | 0.00112075 | 0.33742551 | 0.273 | 0.178 | 1 | 1A |
| AC160336.1 | 4.78E−05 | 0.33560325 | 0.103 | 0.031 | 1 | 1A |
| Bcl2a1b | 6.02E−04 | 0.33447266 | 0.26 | 0.164 | 1 | 1A |
| Selplg | 1.17E−04 | 0.33425329 | 0.331 | 0.206 | 1 | 1A |
| Sh3bgrl3 | 4.89E−07 | 0.33400659 | 0.655 | 0.467 | 0.01519397 | 1A |
| Il18r1 | 1.83E−04 | 0.33226619 | 0.116 | 0.044 | 1 | 1A |
| Gm12840 | 2.40E−04 | 0.32309433 | 0.27 | 0.164 | 1 | 1A |
| Glipr2 | 1.93E−04 | 0.32170466 | 0.113 | 0.042 | 1 | 1A |
| Myl6 | 4.52E−07 | 0.31950379 | 0.612 | 0.417 | 0.01404891 | 1A |
| Ago2 | 0.00315701 | 0.31850509 | 0.105 | 0.05 | 1 | 1A |
| Tmem160 | 0.00308335 | 0.31338957 | 0.151 | 0.086 | 1 | 1A |
| Reep5 | 1.33E−04 | 0.3131882 | 0.262 | 0.15 | 1 | 1A |
| Ifrd1 | 1.26E−04 | 0.31275088 | 0.318 | 0.2 | 1 | 1A |
| Churc1 | 0.00214117 | 0.31158077 | 0.146 | 0.078 | 1 | 1A |
| Irf2bp2 | 7.66E−04 | 0.30727337 | 0.167 | 0.089 | 1 | 1A |
| Anxa6 | 0.00153799 | 0.30678122 | 0.223 | 0.139 | 1 | 1A |
| Ybx3 | 0.00734901 | 0.30585192 | 0.137 | 0.081 | 1 | 1A |
| Samsn1 | 1.70E−05 | 0.3056115 | 0.351 | 0.206 | 0.52786519 | 1A |
| Bhlhe40 | 3.84E−07 | 0.3048086 | 0.673 | 0.456 | 0.01192787 | 1A |
| AC149090.1 | 0.00281847 | 0.30389715 | 0.168 | 0.097 | 1 | 1A |
| Tgfb1 | 2.71E−04 | 0.30387942 | 0.216 | 0.117 | 1 | 1A |
| Otulin | 0.00732043 | 0.3028837 | 0.106 | 0.056 | 1 | 1A |
| 2810474O19Rik | 0.00792142 | 0.29987921 | 0.164 | 0.103 | 1 | 1A |
| Hist1h1c | 0.0040961 | 0.29938665 | 0.149 | 0.083 | 1 | 1A |
| Chd4 | 0.00289298 | 0.29450907 | 0.195 | 0.119 | 1 | 1A |
| Rnf19b | 0.00178859 | 0.29378472 | 0.19 | 0.111 | 1 | 1A |
| Tnfaip3 | 1.60E−06 | 0.29359386 | 0.666 | 0.503 | 0.049755 | 1A |
| Actg1 | 1.47E−07 | 0.29160416 | 0.795 | 0.642 | 0.00455281 | 1A |
| Gm47283 | 0.00346656 | 0.29110079 | 0.294 | 0.203 | 1 | 1A |
| Arf6 | 0.00689906 | 0.2886468 | 0.153 | 0.092 | 1 | 1A |
| Plk3 | 0.00425271 | 0.28542972 | 0.147 | 0.083 | 1 | 1A |
| Msn | 1.10E−04 | 0.28398183 | 0.226 | 0.119 | 1 | 1A |
| Ptp4a2 | 9.80E−04 | 0.28282153 | 0.298 | 0.194 | 1 | 1A |
| Cd69 | 2.06E−05 | 0.28226347 | 0.348 | 0.206 | 0.63921425 | 1A |
| Runx3 | 0.00298249 | 0.28216358 | 0.246 | 0.161 | 1 | 1A |
| Arpc5 | 0.00708465 | 0.28123011 | 0.181 | 0.114 | 1 | 1A |
| Bag1 | 0.0085123 | 0.27686508 | 0.14 | 0.083 | 1 | 1A |
| Mrpl33 | 2.57E−04 | 0.27442638 | 0.255 | 0.147 | 1 | 1A |
| Csf1 | 0.0025607 | 0.27238645 | 0.126 | 0.064 | 1 | 1A |
| Adam8 | 0.0075087 | 0.26667539 | 0.106 | 0.056 | 1 | 1A |
| Itk | 6.87E−04 | 0.26563412 | 0.24 | 0.142 | 1 | 1A |
| Rsbnl1 | 0.00575868 | 0.26499732 | 0.175 | 0.108 | 1 | 1A |
| Pcbp1 | 0.00712247 | 0.26452322 | 0.12 | 0.067 | 1 | 1A |
| Esyt1 | 0.00164063 | 0.26200588 | 0.149 | 0.078 | 1 | 1A |
| Akap13 | 4.38E−04 | 0.26000274 | 0.461 | 0.344 | 1 | 1A |
| Ptp4a1 | 0.00520598 | 0.25643297 | 0.122 | 0.064 | 1 | 1A |
| Lcp1 | 3.76E−04 | 0.25426737 | 0.28 | 0.172 | 1 | 1A |
| Dok2 | 0.0065413 | 0.2540281 | 0.139 | 0.081 | 1 | 1A |
| Pdcd4 | 0.00169261 | −0.2971226 | 0.423 | 0.475 | 1 | 1A |
| Zfp36l2 | 0.0064188 | −0.3057181 | 0.402 | 0.458 | 1 | 1A |
| Ccl5 | 7.31E−04 | −0.3200511 | 0.269 | 0.358 | 1 | 1A |
| Rnaset2b | 0.00353012 | −0.4318074 | 0.099 | 0.156 | 1 | 1A |
| Gm8369 | 0.00350461 | −0.5406211 | 0.061 | 0.108 | 1 | 1A |
| Ndufa4 | 2.85E−04 | −0.5744307 | 0.088 | 0.156 | 1 | 1A |
| Ifi27l2a | 6.50E−07 | −0.6389115 | 0.236 | 0.364 | 0.02017563 | 1A |

Although Id3 loss did not affect the molecular features of tissue invasiveness and residence of C1A cells, it caused significantly decreased activity of gene programs associated with CD28 costimulatory signaling and PI3K/AKT signaling FIG. 5E), without reducing CD28 expression (FIG. 13C). This agrees with other studies showing that in the GVHD target tissues, activation of T cell PD-1 by tissue PD-L1 causes inhibition of AKT-mTOR pathway and depletion of GM-CSF-producing Th1 cells.²⁷

Id3 reduces the ChrAcc to TFs critical for PD-1 expression in Th1 cells. To understand the epigenetic effects by which Id3 loss results in activation of gene programs promoting PD-1 expression and effector differentiation, we performed ATAC-seq analysis. Analysis of ChrAcc diversification showed, as defined as ±3.0 kb sequence flanking transcription start site (TSS), at least 3-fold or higher enrichment between call peaks and averaged genomic background. Id3-cKO Th1 cells had 5153 ChrAcc sites more accessible but only 1131 ChrAcc sites less accessible (FIG. 14A). These opened ChrAcc sites are located at the promoters, exons and introns of genes (FIG. 15A), suggesting that Id3 reduces ChrAcc at diverse regulator loci in Th1 cells.

When combining these opened ChrAcc sites with those DEGs detected by RNA-seq analysis, we found a total of 1444 ChrAcc sites distributed at the promoter, exon and intron regions of 253 DEGs (FIG. 15B). These DEGs overlapped with opening ChrAcc in Th1 cells were enriched for gene programs that promote effector differentiation and function, such as ‘regulation of immune effector process’ and ‘Th1 cell cytokine production’ (FIG. 6A). They were also enriched for genes critical for transcription regulation, including ‘protein localization to nucleus’ and ‘regulation of DNA-binding transcription factor activity’ (FIG. 6A).

The ChrAcc sites opened in Id3-cKO Th1 cells were enriched for motifs of TFs downstream from the TCR and cytokine signals, such as Zinc-Finger TFs, AP-1, ETS1, bHLH, Stat3 and NFATC1 (FIG. 6B). RNA-seq analysis revealed that Id3 deficiency resulted in increased transcription of AP-1 (Fos, Jun, and Junb), NFAT (Nfatc2), Tox (Tox2/4) and Stat3 (FIG. 15B). Among them, NFATc1, AP-1 and Stat3 contribute to induction of Pdcdl transcription.³⁷⁻³⁹ ATAC-seq identified enhanced accessibility in the intron 1, C-region and −28 kb upstream of the TSS of Pdcdl gene in Id3-cKO Th1 cells (FIG. 6C). C-region is a critical regulatory locus where NFATc1 binds for initiating Pdcdl transcription.³⁷ The upstream −28 kb is where Stat3, NFATc1, Stat4 and CTCF bind to maintain Pdcdl expression through forming constitutively interacting chromatin loop.⁴⁰ These data suggest that Id3 remodels ChrAcc across the Pdcdl regulatory loci to restrict its expression.

Inhibiting ID3 in human T cells reduces xeno-GVHD but preserves anti-leukemia activity. Finally, we examined whether ID3 ablation on human T cells may impair their induction of xeno-GVHD. CRISPR-Cas9/ID3-specific gRNA complex was delivered into human T cells as described.⁴¹ The genomic targeting efficacy was >90% (FIG. 16A). ID3-CRISPR-KO human T cells were collected after 7 to 9 days of culture and injected (i.v.) into NSG mice. Mice receiving T cell-depleted peripheral blood mononuclear cells (TCD-PBMCs) did not develop GVHD (FIG. 7A). Transfer of scrambled gRNA-transduced T cells (WT) induced severe GVHD, with 80% of mortality by 70 days after transplantation (FIG. 7A-B). In contrast, 71% of NSG mice receiving ID3-CRISPR-KO T cells were protected from severe xeno-GVHD and survived over 80 days (FIG. 7A-B). Notably, ID3 ablation led to increased fraction of PD-1⁺ T cells infiltrating the liver (FIG. 7C-D), resembling the phenotype of murine Id3-cKO T cells.

To examine the impact of ID3-KO on human T cell-mediated anti-leukemia effects, we used CD19-CAR-T cells whose anti-B cell leukemia activity can be convincingly tracked.^{42,43} We intravenously injected Raji cells expressing Firefly Luciferase-eGFP to NSG mice. Three days later, WT and Id3-KO CD19-CAR-T cells were injected in these mice. Both WT and ID3-KO CAR-T cells showed potent effects on eliminating Raji cell leukemia, significantly improving the overall survival of leukemia mice (FIG. 7E-G). Thus, inhibiting T cell ID3 is effective for reducing GVHD without compromising anti-leukemia activity.

### Discussion

This study has demonstrated that Id3 protects tissue-infiltrating alloreactive T cells against the PD-1/PD-L1 pathway-mediated suppression. Id3 limits Th1 cell expression of PD-1 and maintains tissue-infiltrating PD-1⁺ polyfunctional Th1 cells and PD-1⁺TCF-1+ progenitor-like T cells. Id3 reduces chromatin accessibility of TFs that drive effector differentiation and induce PD-1 transcription. CRISPR/Cas9 KO of ID3 in human T cells inhibits their induction of xeno-GVHD, but reassuringly ID3 ablated CD19-directed CAR T cells retained their anti-leukemia activity. Thus, T cell ID3 may be an effective target to reduce GVHD in nonhematopoietic tissues.

Expansion and function of organ-infiltrating donor T cells are critical to GVHD development.¹⁻³ When infiltrating the GVHD target tissues, alloreactive T cells acquire transcriptomic and protein profiles associated with local tissues.^{1,2} How these processes are regulated remains largely unknown.³ Sacirbegovic et al. discovered that tissue-resident CD39^{lo}TCF-1⁺ progenitor-like cells possessed greater ability than CD39^{hi}TCF-1⁻ CD4⁺ effector cells to produce secondary effector cells upon antigen rechallenge.² We found that loss of Id3 led to depletion of PD-1⁺TCF-1⁺ progenitor-like cells in the liver. Thus, Id3 controls intrinsic transcriptional programs critical for maintaining expansion and function of tissue-infiltrating alloreactive T cells.

Our findings are important for understanding T cell-mediated pathogenic inflammation. The protective effect of Id3 on alloreactive T cell against PD-1 suppression is likely associated with the GVHD tissue microenvironment. During GVHD, PD-L1 in the target tissue acts as a molecular shield to limit T cell attacks. Parenchymal cells produced high levels of PD-L1 in response to IFN-γ produced by alloreactive T cells, and the interaction between PD-L1 and PD-1 becomes dominant in GVHD target tissues relative to that in lymphoid tissues.^{25,27,31,44} Previous studies suggest that the PD-1-mediated suppression is insufficient to inhibit GVHD.³⁰⁻³¹ We identify that Id3 controls gene programs that enable alloreactive T cells resistance against the PD-1 effect. Id3 ablation led to increased PD-1 expression in alloreactive T cells and impaired persistence of tissue-infiltrating polyfunctional Th1 cells.

PD-L1 activation of PD-1 leads to impaired T cell responses via a mechanism of inactivating CD28 signaling and PI3K-AKT signaling activity.^{24,25,36} We observed that Id3 loss resulted in significantly decreased activity of gene programs associated with CD28 signaling and PI3K/AKT signaling, and enhanced frequency of Pdcdi-expressing polyfunctional Th1 cells. Activated T cells become sensitive to the PD-1 pathway-mediated suppression under suboptimal levels of CD28-costimulation.²⁴ Since nonhematopoietic tissues are known to produce low levels of CD28 ligand CD86, Id3 suppression of PD-1 expression in polyfunctional Th1 cells may reduce their sensitivity to PD-L1-induced suppression in the local tissues.

We identify for the first time that Id3 modulates the ChrAcc for TFs that drive Pdcdl transcription. Many TFs have been found to activate PD-1, including NFAT, c-Fos/AP-1 and Stat3.³⁴⁻³⁷⁻⁴⁵ NFAT2 and other TFs (e.g., Tox and Nr4a) can activate PD-1 transcription in T cells.³⁷⁻³⁹⁻⁴⁶ We found that Id3 may play important roles in orchestrating the expression of these TFs and their ChrAcc sites at the Pdcdl locus. Id3 loss resulted in upregulation of transcripts encoding TFs (NFAT, Tox2 and AP-1), and opening of chromatin sites enriched in motifs for TFs (NFAT, AP-1 and STAT3). These Id3-mediated mechanisms add together to orchestrate the expression and function of TFs that promote T cell dysfunction.

The finding that Id3 sustains tissue-infiltrating Th1 cells and TCF-1+ progenitor-like T cells provides novel insights into GVHD pathogenicity. Stimulation by alloantigens, especially those derived from non-hematopoietic tissues, causes alloreactive T cell dysfunction.⁴⁷⁻⁴⁹ Interactions between donor T cells and non-hematopoietic cells may block programming of memory precursors required for producing secondary effectors.⁴⁹ Recent studies demonstrate that those tissue-infiltrating alloreactive T cells can be re-programmed by the local tissues, acquiring the ability to persist during the GVHD process.¹⁻³ We propose that Id3 controls the gene program that enables alloreactive T cells to deflect persistent alloantigen-induced dysfunction.

As CAR-T cell therapy is increasingly used to treat recurrent hematological malignancies, GVHD could potentially be a major challenge hindering allogeneic or third-party CAR T cell therapy. In a preclinical study, 4-1BB-costimulated CAR-T cells increased the occurrence of GVHD.⁵⁰ A universal “off-the-shelf” CAR-T cells remains a goal: such products can be readily available, provide a more consistent product, and improved access to the therapy.⁵¹ Targeting ID3 in CAR T cells may be a potential approach to reduce alloreactivity but retains anti-leukemia activity. A recent study suggested that ID3 is involved in mediating CAR-T cell dysfunction.⁴¹ In response to a persistent CD19⁺ tumor, CD8⁺CD19-CAR-T cells (expressing 4-1BB domain) underwent a NK-like CAR T cell transition. These transitioned NK-like CAR-T cells were phenotypically exhausted cells with attenuated anti-tumor effects.⁴¹ However, whether those CAR T cells expressing low levels of ID3 may diminish during interacting with tumor cells is not reported. Since ID3 is required for maintaining tissue-infiltrating GVHD T cells, we suggest that ID3 might be also important for maintaining tumor-reactive T cells that infiltrate solid tumors. Unlike solid tumors, leukemia mostly occurs in lymphoid-hematopoietic organs where polyfunctional Th1 cells are not affected by the absence of ID3. This may explain that deletion of ID3 from CD19-CAR T cells did not significantly impair anti-leukemia activity.

In summary, we have identified a key role for Id3 in fostering the maintenance of tissue infiltrating polyfunctional effector T cells during GVHD. Targeting ID3 in allogeneic CAR T cells preserves their potent anti-leukemia immunity while reducing GVHD. Thus, targeting Id3 may represent a unique approach to reducing GVHD, and may be more broadly applicable to other inflammatory or autoimmune disorders. Furthermore, given the crucial role of the PD-1 pathway in repressing tumor immunity,⁵² programming Id3 in tumor-reactive T cells may enhance their therapeutic efficacy by rendering them resistant to the PD-1 pathway-mediated suppression.

### REFERENCES

1. Tkachev V, Kaminski J, Potter E L, et al. Spatiotemporal single-cell profiling reveals that invasive and tissue-resident memory donor CD8(+) T cells drive gastrointestinal acute graft-versus-host disease. Sci Transl Med. 2021; 13(576): eabc0227.
2. Sacirbegovic F, Gunther M, Greco A, et al. Graft-versus-host disease is locally maintained in target tissues by resident progenitor-like T cells. Immunity. 2023 56 (2): 369-85.
3. Santos-e-Sousa, P, Cire S, Conlan T, et al. Peripheral tissues reprogram CD8+ T cells for pathogenicity during graft-versus-host disease. JCI Insight. 2018; 3(5): e97011.
4. Yu X, Ma H, Li B, et al. A Novel RIPK1 Inhibitor Reduces GVHD in Mice via a Non-immunosuppressive Mechanism that Restores Intestinal Homeostasis. Blood. 2022.
5. Takashima S, Martin M L, Jansen S A, et al. T cell-derived interferon-gamma programs stem cell death in immune-mediated intestinal damage. Sci Immunol. 2019; 4(42).
6. Zeiser R, Blazar B R. Acute Graft-versus-Host Disease—Biologic Process, Prevention, and Therapy. The New England journal of medicine. 2017; 377(22):2167-2179.
7. Ullrich E, Abendroth B, Rothamer J, et al. BATF-dependent IL-7RhiGM-CSF+ T cells control intestinal graft-versus-host disease. J Clin Invest. 2018; 128(3):916-930.
8. Hill G R, Betts B C, Tkachev V, Kean L S, Blazar B R. Current Concepts and Advances in Graft-Versus-Host Disease Immunology. Annu Rev Immunol. 2021; 39:19-49.
9. Piper C, Zhou V, Komorowski R, et al. Pathogenic Bhlhe40+GM-CSF+CD4+ T cells promote indirect alloantigen presentation in the GI tract during GVHD. Blood. 2020; 135(8):568-581.
10. Engel J A, Lee H J, Williams C G, et al. Single-cell transcriptomics of alloreactive CD4+ T cells over time reveals divergent fates during gut graft-versus-host disease. JCI Insight. 2020; 5(13).
11. Jones-Mason M E, Zhao X, Kappes D, Lasorella A, Iavarone A, Zhuang Y. E protein transcription factors are required for the development of CD4(+) lineage T cells. Immunity. 2012; 36(3):348-361.
12. Lauritsen J P, Wong G W, Lee S Y, et al. Marked induction of the helix-loop-helix protein Id3 promotes the gammadelta T cell fate and renders their functional maturation Notch independent. Immunity. 2009; 31(4):565-575.
13. Zhang B, Lin Y Y, Dai M, Zhuang Y. Id3 and Id2 act as a dual safety mechanism in regulating the development and population size of innate-like gammadelta T cells. J Immunol. 2014; 192(3):1055-1063.
14. Verykokakis M, Krishnamoorthy V, Iavarone A, Lasorella A, Sigvardsson M, Kee B L. Essential functions for ID proteins at multiple checkpoints in invariant NKT cell development. J Immunol. 2013; 191(12):5973-5983.
15. Yang C Y, Best J A, Knell J, et al. The transcriptional regulators Id2 and Id3 control the formation of distinct memory CD8+ T cell subsets. Nat Immunol. 2011; 12(12):1221-1229.
16. Ji Y, Pos Z, Rao M, et al. Repression of the DNA-binding inhibitor Id3 by Blimp-1 limits the formation of memory CD8+ T cells. Nat Immunol. 2011; 12(12):1230-1237.
17. Shan Q, Hu S S, Zhu S, et al. Tcf1 preprograms the mobilization of glycolysis in central memory CD8(+) T cells during recall responses. Nat Immunol. 2022; 23(3):386-398.
18. Shaw L A, Deng T Z, Omilusik K D, Takehara K K, Nguyen Q P, Goldrath A W. Id3 expression identifies CD4(+) memory Th1 cells. Proc Natl Acad Sci USA. 2022; 119(29):e2204254119.
19. Utzschneider D T, Gabriel S S, Chisanga D, et al. Early precursor T cells establish and propagate T cell exhaustion in chronic infection. Nat Immunol. 2020; 21(10):1256-1266.
20. Wysocki C A, Panoskaltsis-Mortari A, Blazar B R, Serody J S. Leukocyte migration and graft-versus-host disease. Blood. 2005; 105(11):4191-4199.
21. Reshef R, Luger S M, Hexner E O, et al. Blockade of lymphocyte chemotaxis in visceral graft-versus-host disease. The New England journal of medicine. 2012; 367(2):135-145.
22. Liu X, Chen X, Zhong B, et al. Transcription factor achaete-scute homologue 2 initiates follicular T-helper-cell development. Nature. 2014; 507(7493):513-518.
23. Maruyama T, Li J, Vaque J P, et al. Control of the differentiation of regulatory T cells and T(H)17 cells by the DNA-binding inhibitor Id3. Nat Immunol. 2011; 12(1):86-95.
24. Freeman G J, Long A J, Iwai Y, et al. Engagement of the PD-1 immunoinhibitory receptor by a novel B7 family member leads to negative regulation of lymphocyte activation. J Exp Med. 2000; 192(7):1027-1034.
25. Sharpe A H, Pauken K E. The diverse functions of the PD1 inhibitory pathway. Nature reviews Immunology. 2018; 18(3):153-167.
26. Patankar J V, Becker C. Cell death in the gut epithelium and implications for chronic inflammation. Nat Rev Gastroenterol Hepatol. 2020; 17(9):543-556.
27. Song Q, Wang X, Wu X, et al. Tolerogenic anti-IL-2 mAb prevents graft-versus-host disease while preserving strong graft-versus-leukemia activity. Blood. 2021; 137(16):2243-2255.
28. Tugues S, Amorim A, Spath S, et al. Graft-versus-host disease, but not graft-versus-leukemia immunity, is mediated by GM-CSF-licensed myeloid cells. Sci Transl Med. 2018; 10(469).
29. Blazar B R, Carreno B M, Panoskaltsis-Mortari A, et al. Blockade of programmed death-1 engagement accelerates graft-versus-host disease lethality by an IFN-gamma-dependent mechanism. J Immunol. 2003; 171(3):1272-1277.
30. Deng R, Cassady K, Li X, et al. B7H1/CD80 interaction augments PD-1-dependent T cell apoptosis and ameliorates graft-versus-host disease. J Immunol. 2015; 194(2):560-574.
31. Ni X, Song Q, Cassady K, et al. PD-L1 interacts with CD80 to regulate graft-versus-leukemia activity of donor CD8⁺ T cells. J Clin Invest. 2017; 127(5):1960-1977.
32. Herbaux C, Gauthier J, Brice P, et al. Efficacy and tolerability of nivolumab after allogeneic transplantation for relapsed Hodgkin lymphoma. Blood. 2017; 129(18):2471-2478.
33. Zhang Y, Joe G, Hexner E, Zhu J, Emerson S G. Alloreactive memory T cells are responsible for the persistence of graft-versus-host disease. J Immunol. 2005; 174(5):3051-3058.
34. Patsoukis N, Wang Q, Strauss L, Boussiotis V A. Revisiting the PD-1 pathway. Sci Adv. 2020; 6(38).
35. Zhang Y, Hexner E, Frank D, Emerson S G. CD4⁺ T cells generated de novo from donor hemopoietic stem cells mediate the evolution from acute to chronic graft-versus-host disease. J Immunol. 2007; 179(5):3305-3314.
36. Hui E, Cheung J, Zhu J, et al. T cell costimulatory receptor CD28 is a primary target for PD-1-mediated inhibition. Science. 2017; 355(6332):1428-1433.
37. Oestreich K J, Yoon H, Ahmed R, Boss J M. NFATc1 regulates PD-1 expression upon T cell activation. J Immunol. 2008; 181(7):4832-4839.
38. Xiao G, Deng A, Liu H, Ge G, Liu X. Activator protein 1 suppresses antitumor T-cell function via the induction of programmed death 1. Proc Natl Acad Sci USA. 2012; 109(38):15419-15424.
39. Khan O, Giles J R, McDonald S, et al. TOX transcriptionally and epigenetically programs CD8(+) T cell exhaustion. Nature. 2019; 571(7764):211-218.
40. Austin J W, Lu P, Majumder P, Ahmed R, Boss J M. STAT3, STAT4, NFATc1, and CTCF regulate PD-1 through multiple novel regulatory regions in murine T cells. J Immunol. 2014; 192(10):4876-4886.
41. Good C R, Aznar M A, Kuramitsu S, et al. An NK-like CAR T cell transition in CAR T cell dysfunction. Cell. 2021; 184(25):6081-6100 e6026.
42. Barrett D M, Singh N, Porter D L, Grupp S A, June C H. Chimeric antigen receptor therapy for cancer. Annu Rev Med. 2014; 65:333-347.
43. Porter D L, Levine B L, Kalos M, Bagg A, June C H. Chimeric antigen receptor-modified T cells in chronic lymphoid leukemia. New England Journal of Medicine. 2011; 365(8):725-733.
44. Li X, Deng R, He W, et al. Loss of B7-H1 expression by recipient parenchymal cells leads to expansion of infiltrating donor CD8+ T cells and persistence of graft-versus-host disease. J Immunol. 2012; 188(2):724-734.
45. Bally A P, Austin J W, Boss J M. Genetic and Epigenetic Regulation of PD-1 Expression. J Immunol. 2016; 196(6):2431-2437.
46. Seo H, Chen J, Gonzalez-Avalos E, et al. TOX and TOX2 transcription factors cooperate with NR4A transcription factors to impose CD8(+) T cell exhaustion. Proc Natl Acad Sci USA. 2019; 116(25):12410-12415.
47. Zhou M, Sacirbegovic F, Zhao K, Rosenberger S, Shlomchik W D. T cell exhaustion and a failure in antigen presentation drive resistance to the graft-versus-leukemia effect. Nat Commun. 2020; 11(1):4227.
48. Asakura S, Hashimoto D, Takashima S, et al. Alloantigen expression on non-hematopoietic cells reduces graft-versus-leukemia effects in mice. The Journal of clinical investigation. 2010; 120(7):2370-2378.
49. Flutter B, Edwards N, Fallah-Arani F, et al. Nonhematopoietic antigen blocks memory programming of alloreactive CD8+ T cells and drives their eventual exhaustion in mouse models of bone marrow transplantation. The Journal of clinical investigation. 2010; 120(11):3855-3868.
50. Ghosh A, Smith M, James S E, et al. Donor CD19 CAR T cells exert potent graft-versus-lymphoma activity with diminished graft-versus-host activity. Nat Med. 2017.
51. Tang T C Y, Xu N, Nordon R, Haber M, Micklethwaite K, Dolnikov A. Donor T cells for CAR T cell therapy. Biomark Res. 2022; 10(1):14.
52. Lee J, Ahn E, Kissick H T, Ahmed R. Reinvigorating Exhausted T Cells by Blockade of the PD-1 Pathway. For Immunopathol Dis Therap. 2015; 6(1-2):7-17.

### Example 2

Ectopic ID3 expression (ID3^{OE}) enhances anti-leukemia activity of chimeric antigen receptor (CAR) T cells. CAR T cell therapy is increasingly used to treat hematological malignancies without GVHD. However, about 50% of patients with B cell malignancies treated with CD19 CAR T cells relapse after the therapy. CAR T cell efficacy is often limited by loss of T cell stemness, poor expansion capacity, and exhaustion when exposed to persistent tumor antigens.⁶⁸⁻⁷³ Given the critical dual functions of Id3 in alloreactive T cells, i.e., restraining excessive PD-1 expression and promoting persistence of PD-1⁺CD4⁺Th1 cells and stem-like Tcf1+T_{PRO} cells, we reasoned that human CAR T cells might require ID3 to achieve an optimal therapeutic efficacy.

Human CD19-CAR T cells whose anti-B cell leukemia activity can be convincingly tracked were used.⁷⁴⁻⁷⁵ Human T cells (CD4⁺ and CD8⁺) were transduced with a lentivirus encoding CD19-4-1BB-CAR (BBz CAR), together with a lentivirus encoding ID3 or GFP, to produce ID3E-BBz-CAR T cells or Con-BBz-CAR T cells, respectively (FIG. 8A). Infusion of ID3E-BBz-CAR T cells showed a greater ability to eliminate Raji leukemia compared to Con-CAR T cells, markedly improving the survival rate (FIG. 8B-D). Thus, ID3^{OE} in BBz-CAR T cells dramatically augments their anti-tumor activity.

Our findings do not support a recent study by Good, et al.⁷⁰ Using an in vitro culture system, they report that ID3 expression is associated with the transition of exhausted CAR T cells into dysfunctional NK-like cells.⁷⁰ However, their studies do not examine the role of ID3 in human CAR T cells in vivo using any preclinical models.⁷⁰

### REFERENCES

68. Weber, E. W. et al. Transient rest restores functionality in exhausted CAR-T cells through epigenetic remodeling. Science 372 (2021). https://doi.org:10.1126/science.aba1786.
69. Wang, Y. et al. Low-dose decitabine priming endows CAR T cells with enhanced and persistent antitumour potential via epigenetic reprogramming. Nat Commun 12, 409 (2021). https://doi.org:10.1038/s41467-020-20696-x.
70. Good, C. R. et al. An NK-like CAR T cell transition in CAR T cell dysfunction. Cell 184, 6081-6100 e6026 (2021). https://doi.org:10.1016/j.cell.2021.11.016.
71. Guedan, S. et al. Single residue in CD28-costimulated CAR-T cells limits long-term persistence and antitumor durability. The Journal of clinical investigation 130, 3087-3097 (2020). https://doi.org:10.1172/JCI133215.
72. Seo, H. et al. TOX and TOX2 transcription factors cooperate with NR4A transcription factors to impose CD8(+) T cell exhaustion. Proc Natl Acad Sci USA 116, 12410-12415 (2019).
73. Long, A. H. et al. 4-1BB costimulation ameliorates T cell exhaustion induced by tonic signaling of chimeric antigen receptors. Nat Med 21, 581-590 (2015). https://doi.org:10.1038/nm.3838.
74. Davila, M L and Brentjens, RJ. CD-19 Targeted CAR T cells as novel cancer immunotherapy for relapsed or refractory B-cell acute lymphoblastic leukemia. Clin. Adv. Hematol. Oncol. (2016) 14 (10): 802-808.
75. Cappell, K. M. et al. Long-Term Follow-Up of Anti-CD19 Chimeric Antigen Receptor T-Cell Therapy. J. Clin. Oncol. (2020) 38 (32): 3805-3815.

### Example 3. Ectopic Expression of ID30E in Human Chimeric Antigen Receptor (CAR) T Cells Enhances Anti-Leukemia Activity

CAR T cell therapy is increasingly used to treat hematological malignancies without GVHD. However, about 50% of patients with B cell malignancies treated with CD19 CAR T cells relapse after the therapy. CAR T cell efficacy is often limited by loss of T cell stemness, poor expansion capacity, and exhaustion when exposed to persistent tumor antigens.¹⁻⁶ Given the critical dual functions of Id3 in alloreactive T cells, i.e., restraining excessive PD-1 expression and promoting persistence of PD-1+CD4+Th1 cells and stem-like Tcf1+ progenitor T (T_{PRO}) cells, we reasoned that human CAR T cells might require ID3 to achieve an optimal therapeutic efficacy.

To address it, we used human CD19-CAR T cells whose anti-B cell leukemia activity can be convincingly tracked.⁷⁻⁸ We transduced human T cells (CD4⁺ and CD8⁺) with a lentivirus encoding CD19-4-1BB-CAR (BBz CAR), together with a lentivirus encoding ID3 or GFP, to produce ID3⁰E-BBz-CAR T cells or Con-BBz-CAR T cells (control), respectively (FIG. 18A, one plasmid system). Infusion of ID3^{OE}-BBz-CAR T cells showed a greater ability to eliminate human xenograft Raji leukemia compared to Con-CAR T cells, markedly improving the survival rate (FIG. 18B, FIG. 18C, FIG. 18D, FIG. 18E). Thus, ID3^{OE} in BBz-CAR T cells drastically augments their anti-tumor activity. Similar results were observed using two plasmids system (FIG. 18C, low panel).

We observed that human ID3^{OE}-CAR T cells had enhanced capability to persist and expand during and after leukemia control. ID3^{OE}-CAR T cells had greater capacity to expand in vivo (FIG. 19A), produced significantly fewer frequency of terminal exhausted T cells (term-TEX, PD-1+TIM3+) but higher frequency of precursor-TEX (PD-1+TIM3−) cells than Con-CAR T cells during leukemia control (FIG. 19B, FIG. 19C). Upon ex vivo culture in the presence of homeostatic cytokines (IL-2, IL-7 and IL-15), these ID3^{OE}-CAR T cells produced high frequency of CD62L+CD45RA− central memory phenotype (FIG. 19D) and acquired enhanced ability to produce IL-2 and to proliferate upon antigen rechallenge compared to Con-CAR T cells (FIG. 19E-FIG. 19F).

We discovered that human ID3^{OE}-CAR T cells had augmented memory protection against tumor challenges in human xenograft leukemia NSG mice. Those leukemia mice who survived ID3^{OE}-CAR T cells over 80 days without leukemia were rechallenged by subcutaneous injection of Raji cells, with age-matched NSG mice as control (FIG. 20A). ID3^{OE}-CAR T cell-treated leukemia mice prevented the growth of lymphoma under the skin with 7/9 of them surviving without tumor by 55 days after tumor challenge (FIG. 20B, FIG. 20C). In contrast, all control mice died from lymphoma within 35 days after tumor challenge (FIG. 20B, FIG. 20C).

### REFERENCES

1. Locke, F. L., et al. Axicabtagene Ciloleucel as Second-Line Therapy for Large B-Cell Lymphoma. The New England journal of medicine 386, 640-654 (2022).
2. Chong, E. A., Ruella, M., Schuster, S. J. & Lymphoma Program Investigators at the University of, P. Five-Year Outcomes for Refractory B-Cell Lymphomas with CAR T-Cell Therapy. The New England journal of medicine 384, 673-674 (2021).
3. Good, C. R., et al. An NK-like CAR T cell transition in CAR T cell dysfunction. Cell 184, 6081-6100 e6026 (2021).
4. Good, Z., et al. Post-infusion CAR T(Reg) cells identify patients resistant to CD19-CAR therapy. Nat Med 28, 1860-1871 (2022).
5. Weber, E. W., et al. Transient rest restores functionality in exhausted CAR-T cells through epigenetic remodeling. Science 372(2021).
6. Lynn, R. C., et al. c-Jun overexpression in CAR T cells induces exhaustion resistance. Nature 576, 293-300 (2019).
7. Barrett, D. M., Singh, N., Porter, D. L., Grupp, S. A. & June, C. H. Chimeric antigen receptor therapy for cancer. Annu Rev Med 65, 333-347 (2014).
8. Porter, D. L., Levine, B. L., Kalos, M., Bagg, A. & June, C. H. Chimeric antigen receptor-modified T cells in chronic lymphoid leukemia. New England Journal of Medicine 365, 725-733 (2011).

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. An immunotherapy method for treating a recipient subject with a hematologic cancer comprising administering to the recipient subject an activated and expanded purified population of genetically engineered CD3+ T cells derived from a healthy donor,
a. wherein the genetic engineering of the donor CD3+ T cells in vitro reduces expression of Id3, an inhibitor of DNA-binding E-protein transcription factors, by at least 25%, compared to a control; and
b. wherein the method poses a decreased risk of graft versus host reaction while preserving graft versus tumor immunity in the recipient subject.

2. The immunotherapy method according to claim 1, wherein the administering is by infusion.

3. The immunotherapy method according to claim 1, wherein
a. The subject is a mammal; or
b. The subject is a human.

4. The immunotherapy method according to claim 1, wherein the hematologic cancer is a leukemia, a myelodysplastic neoplasm, a myeloma, or a lymphoma.

5. The immunotherapy method according to claim 1 wherein the donor T cells are allogeneic to the recipient subject.

6. The immunotherapy method according to claim 1, wherein
a. the CD3+ T cells are purified from mononuclear cells collected from umbilical cord blood or adult peripheral blood; and
b. the CD3+ T cells comprise CD4+ T cells, CD8+ T cells or both; and
c. the CD3+ T cells comprising the edited Id3 gene are expanded and activated in vitro in presence of a cytokine.

7. The immunotherapy method according to claim 6, wherein the cytokine is selected from the group consisting of IL-2, IL-7, IL-15, IL-18, IL-21, or a combination thereof.

8. The method according to claim 1, wherein the reducing of the expression of the Id3 gene of the CD3+ T cells is accomplished by CRISPR/Cas9.

9. The method according to claim 6, wherein the activated and expanded purified population of genetically engineered CD3+ T cells comprising CD4+ T cells, CD8+ T cells or both comprising the edited Id3 gene is characterized by an improved ability to secrete effector cytokines, an improved cytotoxicity, or both against tumor cells compared to a control population of mononuclear cells.

10. An immunotherapy method for treating a recipient subject with a hematologic cancer comprising
a. genetically engineering a population of CD3+ T cells derived from a healthy donor:
i. to express a chimeric antigen receptor (CAR) that specifically binds a tumor antigen; and
ii. to ectopically express Id3 (Id30E);
b. activating and expanding the purified population of genetically engineered CD3+CAR−, Id^{OE} T-cells of (a); and
c. administering the activated and expanded purified population of genetically engineered population of CD3⁺CAR, Id^{OE} T-cells of (b) wherein the cell population is characterized by:
a lower frequency of terminally exhausted Tcells of phenotype PD-1+TIM3+; or
a higher frequency of progenitor exhausted cells (T_{PEX}) of phenotype PD1+TIM3−; or
an enhanced persistence and enhanced ability to expand in vitro; or
a higher frequency of cells of a central memory cell phenotype (CD62+CD45RA⁻) upon ex vivo culture in IL-2, IL-7 and IL-15; or
an enhanced ability to produce IL-2 and to proliferate upon antigen challenge in ex vivo culture; or
augmented memory protection against tumor challenge in a mouse leukemia model; or
improved overall survival; or
a combination thereof, compared to control CAR-T cells.

11. The immunotherapy method according to claim 10, wherein
a. the genetic engineering to express a CAR comprises transducing the CD3+ T cells with a retroviral vector comprising a nucleic acid encoding a synthetic CAR to stably express the CAR; and
b. the CAR comprises an extracellular antigen recognition domain, a spacer/hinge region and transmembrane domain, and an intracellular signal transduction domain; and
c. the therapeutic dose of the CAR-T cells is about 1×10E6 to 20×10E6 CAR-T cells/m² body surface area.

12. The immunotherapy method according to claim 11, wherein
a. the extracellular antigen recognition domain of the CAR comprising an scFv fragment derived from a monoclonal antibody binds specifically to CD19, CD20, CD22, CD33, or CD30; and
b. the intracellular signal transduction domain of the CAR comprises a CD3ζ activation chain and one or more costimulatory molecules.

13. The immunotherapy method according to claim 12, wherein the costimulatory molecule comprises 4-1BB.

14. The immunotherapy method according to claim 10, further comprising administering a short course of chemotherapy to reduce the T cell population of the subject prior to the administering of the population of CAR-T ID3^{OE} cells.

15. The method according to claim 10, wherein the human CAR-T cells engineered to ectopically express Id3 have an enhanced ability to eliminate tumors compared to a CAR-T cell control that does not ectopically express Id3.

16. The immunotherapy method according to claim 1, wherein the administering is by infusion.

17. The immunotherapy method according to claim 1, wherein
a. The subject is a mammal; or
b. The subject is a human.

18. The immunotherapy method according to claim 10, wherein the hematologic cancer is a leukemia, a myelodysplastic neoplasm, a myeloma, or a lymphoma.

19. The method according to claim 10 wherein the donor T cells are allogeneic to the recipient subject.

20. The method according to claim 10, wherein
a. the CD3+ T cells are purified from mononuclear cells collected from umbilical cord blood or adult peripheral blood;
b. the CD3+ T cells comprise CD4+ T cells, CD8+ T cells or both and
c. the CD3+ T cells are expanded and activated in vitro in presence of a cytokine selected from IL-2, IL-7, IL-15, IL-18, IL-21, or a combination thereof.

21. The method according claim 10, further comprising administering an additional agent.

22. The method according to claim 21, wherein the additional agent comprises:
an approved immune checkpoint inhibitor at a dose standard for the cancer indication, or
rituxuximab (anti-CD20); or
alemtuzumab (antiCD52); or
epratuzumab (anti-CD-22); or
a clinical grade alpha-1-antitrypsin.

23. The method according to claim 22, wherein the immune checkpoint inhibitor is an anti-PD-1 inhibitor; an anti-PD-L1 inhibitor, or an anti-CTLA-4 inhibitor.

24. The method according to claim 23, wherein the anti-PD-1 inhibitor is lambrolizumab/pembrolizumab or nivolumab.

25. The method according to claim 23, wherein the anti-PDL-1 inhibitor is atezolizumab.

26. The method according to claim 23, wherein the anti-CTLA-4 inhibitor is ipilimumab.


SEQUENCE LISTING (7 sequences)

SEQ ID NO: 1 - DNA, 20 bp, synthetic construct
  misc_feature 1..20: note = Synthetic: hID3sgRNA-1
  source 1..20: mol_type = other DNA
tggctaagctgagtgcctct

SEQ ID NO: 2 - DNA, 20 bp, synthetic construct
  misc_feature 1..20: note = Synthetic: hID3crRNA-2
  source 1..20: mol_type = other DNA
tggccagactgcgttccgac

SEQ ID NO: 3 - DNA, 20 bp, synthetic construct
  misc_feature 1..20: note = Synthetic: Neg cntrl crRNA
  source 1..20: mol_type = other DNA
cgttaatcgcgtataatacg

SEQ ID NO: 4 - DNA, 20 bp, synthetic construct
  misc_feature 1..20: note = Synthetic: hID3.PCR F(gDNA)
  source 1..20: mol_type = other DNA
ataaagaggcgtgccttcca

SEQ ID NO: 5 - DNA, 20 bp, synthetic construct
  misc_feature 1..20: note = Synthetic: hID3.PCR. R(gDNA)
  source 1..20: mol_type = other DNA
catccttgcctgggtgttca

SEQ ID NO: 6 - DNA, 18 bp, synthetic construct
  misc_feature 1..18: note = Synthetic: ID3.Seq.R (gDNA)
  source 1..18: mol_type = other DNA
gaaggtgggggccatcag

SEQ ID NO: 7 - DNA, 20 bp, synthetic construct
  misc_feature 1..20: note = Synthetic: ID3.Seq.F (gDNA)
  source 1..20: mol_type = other DNA
ttctctttggggcacctctg